SEQUENCE LISTING (93 sequences)

SEQ ID NO: 1 - protein, 306 aa, Homo sapiens
SVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 2 - protein, 182 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKR

SEQ ID NO: 3 - protein, 52 aa, Homo sapiens
SVAQATSSSGEAPDSITWKPYDAADLDPTENPFDLLDFNQTQPERGDNNLTR

SEQ ID NO: 4 - protein, 488 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 5 - protein, 142 aa, Homo sapiens
ANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKR

SEQ ID NO: 6 - protein, 254 aa, Homo sapiens [sequence omitted]

SEQ ID NO: 7 - protein, 357 aa, Homo sapiens
KYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 8 - DNA, 1071 bp, Homo sapiens
aaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggaggaagaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaaggaagaccaggtggacccaagactgattgacggaaaatgagaattcggatcccccgacctcgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 9 - protein, 354 aa, Homo sapiens
KYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 10 - DNA, 1062 bp, Homo sapiens
aaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 11 - protein, 100 aa, Homo sapiens
KYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKR

SEQ ID NO: 12 - protein, 357 aa, Homo sapiens
KYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTYETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 13 - protein, 357 aa, Homo sapiens
KYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGGQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 14 - protein, 357 aa, Homo sapiens
KYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGIQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 15 - protein, 357 aa, Homo sapiens
KYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGYQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 16 - protein, 357 aa, Homo sapiens
KYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGFQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 17 - DNA, 39 bp, Artificial Sequence
  primer
ctggaacgcaggaagaggaggaagaggatcgtgggaggc

SEQ ID NO: 18 - DNA, 39 bp, Artificial Sequence
  primer
gcctcccacgatcctcttcctcctcttcctgcgttccag

SEQ ID NO: 19 - protein, 18 aa, Artificial Sequence
  MB7
MRWSWIFLLLLSITSANA

SEQ ID NO: 20 - protein, 13 aa, Artificial Sequence
  Etiquette HPC4 de GPAD1 et GPAD2
CEDQVDPRLIDGK

SEQ ID NO: 21 - protein, 356 aa, Artificial Sequence
  GPAD1
SNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 22 - protein, 359 aa, Artificial Sequence
  GPAD2
SNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 23 - protein, 355 aa, Artificial Sequence
  GPAD1 optimise
NKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 24 - protein, 358 aa, Artificial Sequence
  GPAD2 optimise
NKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 25 - protein, 102 aa, Artificial Sequence
  SEQ ID NO11 avec Ser-Asn- en N terminal
SNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKR

SEQ ID NO: 26 - protein, 101 aa, Artificial Sequence
  SEQ ID NO11 avec Asn- en N terminal
NKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKR

SEQ ID NO: 27 - protein, 359 aa, Artificial Sequence
  Exemple de GPAD3
NKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLEGSSGRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 28 - protein, 360 aa, Artificial Sequence
  GPAD3-LC
NKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERGSSGRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 29 - protein, 366 aa, Artificial Sequence
  GPAD3-LL
NKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERGGGGSGGGGSRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 30 - protein, 369 aa, Artificial Sequence
  GPAD3-2F
NKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRGGGGSGGGGSRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 31 - protein, 266 aa, Artificial Sequence
  Chaine lourde de FXa fusionne a SEQ ID NO27
IVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKEDQVDPRLIDGK

SEQ ID NO: 32 - protein, 586 aa, Artificial Sequence
  GPAD2-FX-Fc
SNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 591 aa, Artificial Sequence
  GPAD2-Fcl
SNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 590 aa, Artificial Sequence
  GPAD2-FcLss
NKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 831 aa, Artificial Sequence
  GPAD2-scFcL
SNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 821 aa, Artificial Sequence
  GPAD2-scFcS
SNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 835 aa, Artificial Sequence
  scFcL-GPAD2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 38 - protein, 832 aa, Artificial Sequence
  scFcL-GPAD1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 39 - protein, 226 aa, Artificial Sequence
  Fragment Fc, eventuellement suivi d′une lysine
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - DNA, 1068 bp, Artificial Sequence
  Sequence nucleique codant GPAD1 (SEQ ID NO21)
agcaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 41 - DNA, 1077 bp, Artificial Sequence
  Sequence nucleique codant GPAD2 (SEQ ID NO22)
agcaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggaggaagaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 42 - DNA, 1065 bp, Artificial Sequence
  Sequence nucleique codant GPAD1optimise (SEQ ID NO23)
aacaagtataaagacggagaccagtgtgagactagcccttgccagaaccaggggaagtgtaaagatggactgggcgagtacacctgcacatgtctggagggattcgaaggcaagaattgcgaactgtttactagaaaactgtgtagcctggataacggcgactgcgatcagttctgtcacgaggaacagaattcagtggtctgcagctgtgccaggggatacaccctggccgacaacggcaaggcttgcatccctaccggcccctatccttgtgggaaacagacactggagcggagaaagaggatcgtgggcgggcaggagtgcaaggatggagaatgtccatggcaggccctgctgattaacgaggaaaatgagggcttctgcggaggcactatcctgtccgaattttacattctgaccgccgctcattgtctgtatcaggccaagcggttcaaggtgcgggtcggcgacagaaacacagagcaggaggaagggggagaagctgtgcacgaggtcgaagtggtcatcaagcataatcgcttcactaaagagacctacgactttgatatcgcagtgctgagactgaagacaccaattactttcaggatgaatgtcgcaccagcatgcctgccagagcgagattgggctgaatccaccctgatgacacagaaaactggcattgtgtctgggtttggacggacacacgagaaggggaggcagagcactcgcctgaaaatgctggaagtgccctacgtcgacaggaactcctgtaagctgagctcctctttcatcattacacagaatatgttttgcgcagggtatgacactaagcaggaggatgcctgtcagggagactctggaggacctcacgtgacccgcttcaaagatacttattttgtgaccggaatcgtcagttggggagagggatgcgctcgaaaggggaaatacggaatctataccaaggtgacagcattcctgaaatggattgaccgaagtatgaagacccggggcctgcctaaggctaaatcacatgcaccagaagtgatcacaagttcacccctgaag

SEQ ID NO: 43 - DNA, 1074 bp, Artificial Sequence
  Sequence nucleique codant GPAD2 optimise (SEQ ID NO24)
aacaagtataaagacggagaccagtgtgagactagcccttgccagaaccaggggaagtgtaaagatggactgggcgagtacacctgcacatgtctggagggattcgaaggcaagaattgcgaactgtttactagaaaactgtgtagcctggataacggcgactgcgatcagttctgtcacgaggaacagaattcagtggtctgcagctgtgccaggggatacaccctggccgacaacggcaaggcttgcatccctaccggcccctatccttgtgggaaacagacactggagcggagaaagaggcgcaaacggatcgtgggcgggcaggagtgcaaggatggagaatgtccatggcaggccctgctgattaacgaggaaaatgagggcttctgcggaggcactatcctgtccgaattttacattctgaccgccgctcattgtctgtatcaggccaagcggttcaaggtgcgggtcggcgacagaaacacagagcaggaggaagggggagaagctgtgcacgaggtcgaagtggtcatcaagcataatcgcttcactaaagagacctacgactttgatatcgcagtgctgagactgaagacaccaattactttcaggatgaatgtcgcaccagcatgcctgccagagcgagattgggctgaatccaccctgatgacacagaaaactggcattgtgtctgggtttggacggacacacgagaaggggaggcagagcactcgcctgaaaatgctggaagtgccctacgtcgacaggaactcctgtaagctgagctcctctttcatcattacacagaatatgttttgcgcagggtatgacactaagcaggaggatgcctgtcagggagactctggaggacctcacgtgacccgcttcaaagatacttattttgtgaccggaatcgtcagttggggagagggatgcgctcgaaaggggaaatacggaatctataccaaggtgacagcattcctgaaatggattgaccgaagtatgaagacccggggcctgcctaaggctaaatcacatgcaccagaagtgatcacaagttcacccctgaag

SEQ ID NO: 44 - DNA, 762 bp, Artificial Sequence
  Sequence nucleique codant SEQ ID NO31
atcgtgggcgggcaggagtgcaaggatggagaatgtccatggcaggccctgctgattaacgaggaaaatgagggcttctgcggaggcactatcctgtccgaattttacattctgaccgccgctcattgtctgtatcaggccaagcggttcaaggtgcgggtcggcgacagaaacacagagcaggaggaagggggagaagctgtgcacgaggtcgaagtggtcatcaagcataatcgcttcactaaagagacctacgactttgatatcgcagtgctgagactgaagacaccaattactttcaggatgaatgtcgcaccagcatgcctgccagagcgagattgggctgaatccaccctgatgacacagaaaactggcattgtgtctgggtttggacggacacacgagaaggggaggcagagcactcgcctgaaaatgctggaagtgccctacgtcgacaggaactcctgtaagctgagctcctctttcatcattacacagaatatgttttgcgcagggtatgacactaagcaggaggatgcctgtcagggagactctggaggacctcacgtgacccgcttcaaagatacttattttgtgaccggaatcgtcagttggggagagggatgcgctcgaaaggggaaatacggaatctataccaaggtgacagcattcctgaaatggattgaccgaagtatgaagacccggggcctgcctaaggctaaatcacatgcaccagaagtgatcacaagttcacccctgaag

SEQ ID NO: 45 - DNA, 1758 bp, Artificial Sequence
  Sequence nucleique codant GPAD2-FX-Fc (SEQ ID NO32)
agcaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggaggaagaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaaggacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 46 - DNA, 1773 bp, Artificial Sequence
  Sequence nucleique codant GPAD2-Fcl (SEQ ID NO33)
agcaataagtataaagacggggatcagtgcgagacctccccatgtcagaaccagggcaagtgcaaagacgggctgggagagtacacatgcacttgtctggaggggttcgaaggaaagaattgcgaactgtttacaagaaagctgtgcagcctggataacggcgactgcgatcagttctgtcacgaggaacagaatagtgtggtctgctcatgtgccagggggtacactctggctgacaacggaaaggcatgcatccctactggaccttatccatgtggcaaacagaccctggagcggagaaagaggcgcaaacgcatcgtgggcgggcaggagtgcaaggatggagaatgtccatggcaggctctgctgattaacgaggaaaatgagggcttctgcggaggcacaatcctgagcgagttctacattctgactgccgctcattgtctgtatcaggctaagcggttcaaggtgcgggtcggcgacagaaacaccgagcaggaggaagggggagaagcagtgcacgaggtcgaagtggtcatcaagcataatcgcttcacaaaagagacttacgactttgatatcgccgtgctgagactgaagacccccattacattcaggatgaatgtggcaccagcatgcctgcctgagcgagattgggctgaatcaactctgatgacccagaaaacaggaattgtgagcggctttgggcgaactcacgagaagggcaggcagagcacccgcctgaaaatgctggaagtgccctacgtcgaccggaacagctgtaagctgagctcctctttcatcattacccagaatatgttttgcgccggctatgacacaaagcaggaggatgcttgtcagggggactccggaggacctcatgtgaccagattcaaagatacatattttgtgactggcatcgtctcttggggagaaggctgcgccaggaagggcaaatacgggatctatactaaggtgaccgccttcctgaaatggattgatcgatccatgaagactcggggcctgccaaaggcaaaatctcacgcccccgaagtgatcaccagttcacctctgaaggaacctaagtcttgcgacaaaacccatacatgcccaccttgtccagcacctgaactgctgggaggaccatccgtgttcctgtttccacccaagcccaaagatacactgatgattagtcggacccctgaggtgacatgcgtggtcgtggatgtctcacacgaggacccagaagtgaagtttaactggtacgtggacggcgtggaagtccataatgccaagaccaaacctcgcgaggaacagtacaacagtacatatcgagtcgtgtcagtgctgactgtcctgcaccaggattggctgaacggaaaggagtataagtgcaaagtgagcaataaggctctgccagcacccatcgagaaaacaatttccaaggcaaaaggccagccaagggaaccccaggtgtacactctgcctccaagccgcgatgagctgacaaagaaccaggtgtccctgacttgtctggtcaaagggttctatccctccgacatcgccgtggagtgggaatctaatggacagcctgagaacaattacaagaccacaccccctgtgctggactcagatgggagcttctttctgtattctaagctgactgtggacaaaagtagatggcagcagggaaacgtgttttcttgcagtgtcatgcacgaggccctgcacaatcattacacccagaagtcactgagcctgtccccaggaaag

SEQ ID NO: 47 - DNA, 1770 bp, Artificial Sequence
  Sequence nucleique codant GPAD2-FcLss (SEQ ID NO34)
aataagtataaagacggggatcagtgcgagacctctccttgtcagaaccagggcaagtgcaaagacgggctgggagagtacacatgcacttgtctggaggggttcgaaggaaagaattgcgaactgtttacaagaaaactgtgtagcctggataacggggactgcgatcagttctgtcacgaggaacagaattccgtggtctgctcttgtgcaagggggtacaccctggctgacaacggaaaggcatgcatccctactggaccttatccatgtggcaaacagaccctggagcggagaaagaggcgcaaacgcatcgtgggcgggcaggagtgcaaggatggagaatgtccatggcaggctctgctgattaacgaggaaaatgagggcttctgcggaggcacaatcctgagcgaattttacattctgactgccgctcattgtctgtatcaggctaagcggttcaaggtgcgggtcggcgacagaaacaccgagcaggaggaagggggagaagcagtgcacgaggtcgaagtggtcatcaagcataatcgcttcacaaaagagacttacgactttgatatcgccgtgctgagactgaagacccccattacattcaggatgaatgtggcaccagcatgcctgcctgagcgagattgggctgaatctactctgatgacccagaaaacaggaattgtgagtggctttgggcgaactcacgagaagggcaggcagtctacccgcctgaaaatgctggaagtgccctacgtcgaccggaacagctgtaagctgagctcctctttcatcattacccagaatatgttttgcgccggctatgacacaaagcaggaggatgcttgtcagggggacagcggaggacctcatgtgaccagattcaaagatacatattttgtgactggcatcgtctcctggggagaaggctgcgcaaggaagggcaaatacgggatctatactaaggtgaccgccttcctgaaatggattgatcgatcaatgaagactcggggcctgccaaaggcaaaaagccacgcccccgaagtgatcaccagttcacctctgaaggaaccaaagagctgcgacaaaacccatacatgcccaccttgtccagcacctgaactgctgggaggaccatccgtgttcctgtttccacccaagcccaaagatacactgatgatttcccggacccctgaggtgacatgtgtggtcgtggatgtctctcacgaggacccagaagtgaagtttaactggtacgtggacggcgtggaagtccataatgccaagaccaaaccccgcgaggaacagtacaactccacatatcgagtcgtgtctgtgctgactgtcctgcaccaggattggctgaacggaaaagagtacaagtgcaaagtgagtaataaggctctgccagcacccatcgagaaaacaatttccaaggcaaaaggccagccaagggaaccccaggtgtacactctgcctccaagtcgcgatgagctgacaaagaaccaggtgtcactgacttgtctggtcaaagggttctatccctcagacatcgccgtggagtgggaaagcaatggacagcctgagaacaattacaagaccacaccccctgtgctggactctgatgggagtttctttctgtatagcaagctgactgtggacaaatccagatggcagcagggaaacgtgttttcttgcagtgtcatgcacgaggccctgcacaatcattacacccagaagtcactgagcctgtccccaggaaag

SEQ ID NO: 48 - DNA, 2493 bp, Artificial Sequence
  Sequence nucleique codant GPAD2-scFcL (SEQ ID NO35)
agcaataagtataaagacggggatcagtgcgagacctccccatgtcagaaccagggcaagtgcaaagacgggctgggagagtacacatgcacttgtctggaggggttcgaaggaaagaattgcgaactgtttacaagaaagctgtgcagcctggataacggcgactgcgatcagttctgtcacgaggaacagaatagtgtggtctgctcatgtgccagggggtacactctggctgacaacggaaaggcatgcatccctactggaccttatccatgtggcaaacagaccctggagcggagaaagaggcgcaaacgcatcgtgggcgggcaggagtgcaaggatggagaatgtccatggcaggctctgctgattaacgaggaaaatgagggcttctgcggaggcacaatcctgagcgagttctacattctgactgccgctcattgtctgtatcaggctaagcggttcaaggtgcgggtcggcgacagaaacaccgagcaggaggaagggggagaagcagtgcacgaggtcgaagtggtcatcaagcataatcgcttcacaaaagagacttacgactttgatatcgccgtgctgagactgaagacccccattacattcaggatgaatgtggcaccagcatgcctgcctgagcgagattgggctgaatcaactctgatgacccagaaaacaggaattgtgagcggctttgggcgaactcacgagaagggcaggcagagcacccgcctgaaaatgctggaagtgccctacgtcgaccggaacagctgtaagctgagctcctctttcatcattacccagaatatgttttgcgccggctatgacacaaagcaggaggatgcttgtcagggggactccggaggacctcatgtgaccagattcaaagatacatattttgtgactggcatcgtctcttggggagaaggctgcgccaggaagggcaaatacgggatctatactaaggtgaccgccttcctgaaatggattgatcgatccatgaagactcggggcctgccaaaggcaaaatctcacgcccccgaagtgatcaccagttcacctctgaaggacaaaacccatacatgcccaccttgtccagcacctgaactgctgggaggaccatccgtgttcctgtttccacccaagcccaaagatacactgatgattagtcggacccctgaggtgacatgcgtggtcgtggatgtctcacacgaggacccagaagtgaagtttaactggtacgtggacggcgtggaagtccataatgccaagaccaaacctcgcgaggaacagtacaacagtacatatcgagtcgtgtcagtgctgactgtcctgcaccaggattggctgaacggaaaggagtataagtgcaaagtgagcaataaggctctgccagcacccatcgagaaaacaatttccaaggcaaaaggccagccaagggaaccccaggtgtacactctgcctccaagccgcgatgagctgacaaagaaccaggtgtccctgacttgtctggtcaaagggttctatccctccgacatcgccgtggagtgggaatctaatggacagcctgagaacaattacaagaccacaccccctgtgctggactcagatgggagcttctttctgtattctaagctgactgtggacaaaagtagatggcagcagggaaacgtgttttcttgcagtgtcatgcacgaggccctgcacaatcattacacccagaagtcactgagcctgtccccaggaggaggaggaggaagcggaggaggaggctccgggggaggcgggtctgagcccaagagctccgataaaactcatacctgcccaccctgtcctgctccagaactgctgggaggccctagcgtgttcctgtttcctccaaagccaaaagacacactgatgatttctaggactcccgaggtgacctgcgtggtggtcgatgtcagtcacgaggaccctgaagtgaagttcaactggtacgtggatggagtcgaggtgcacaacgccaagaccaaaccccgggaggaacagtacaacagcacctatagagtggtctccgtgctgacagtcctgcaccaggactggctgaacgggaaggaatacaagtgcaaagtgtccaataaggccctgcccgctcctatcgaaaaaaccatttctaaggctaaaggccagccccgggagccacaggtgtacacactgcccccttctcgggatgaactgaccaagaaccaggtgagtctgacatgtctggtcaaaggcttctatccaagtgacatcgcagtggagtgggaatcaaatgggcagcccgagaacaattacaagactaccccacccgtgctggactccgatggctctttctttctgtattcaaagctgaccgtggacaaaagcagatggcagcaggggaacgtgttcagctgcagtgtcatgcacgaagcactgcacaatcattacactcagaaatcactgtcactgtcacctgga

SEQ ID NO: 49 - DNA, 2463 bp, Artificial Sequence
  Sequence nucleique codant GPAD2-scFcS (SEQ ID NO36)
agcaataagtataaagacggggatcagtgcgagacctccccatgtcagaaccagggcaagtgcaaagacgggctgggagagtacacatgcacttgtctggaggggttcgaaggaaagaattgcgaactgtttacaagaaagctgtgcagcctggataacggcgactgcgatcagttctgtcacgaggaacagaatagtgtggtctgctcatgtgccagggggtacactctggctgacaacggaaaggcatgcatccctactggaccttatccatgtggcaaacagaccctggagcggagaaagaggcgcaaacgcatcgtgggcgggcaggagtgcaaggatggagaatgtccatggcaggctctgctgattaacgaggaaaatgagggcttctgcggaggcacaatcctgagcgagttctacattctgactgccgctcattgtctgtatcaggctaagcggttcaaggtgcgggtcggcgacagaaacaccgagcaggaggaagggggagaagcagtgcacgaggtcgaagtggtcatcaagcataatcgcttcacaaaagagacttacgactttgatatcgccgtgctgagactgaagacccccattacattcaggatgaatgtggcaccagcatgcctgcctgagcgagattgggctgaatcaactctgatgacccagaaaacaggaattgtgagcggctttgggcgaactcacgagaagggcaggcagagcacccgcctgaaaatgctggaagtgccctacgtcgaccggaacagctgtaagctgagctcctctttcatcattacccagaatatgttttgcgccggctatgacacaaagcaggaggatgcttgtcagggggactccggaggacctcatgtgaccagattcaaagatacatattttgtgactggcatcgtctcttggggagaaggctgcgccaggaagggcaaatacgggatctatactaaggtgaccgccttcctgaaatggattgatcgatccatgaagactcggggcctgccaaaggcaaaatctcacgcccccgaagtgatcaccagttcacctctgaaggacaaaacccatacatgcccaccttgtccagcacctgaactgctgggaggaccatccgtgttcctgtttccacccaagcccaaagatacactgatgattagtcggacccctgaggtgacatgcgtggtcgtggatgtctcacacgaggacccagaagtgaagtttaactggtacgtggacggcgtggaagtccataatgccaagaccaaacctcgcgaggaacagtacaacagtacatatcgagtcgtgtcagtgctgactgtcctgcaccaggattggctgaacggaaaggagtataagtgcaaagtgagcaataaggctctgccagcacccatcgagaaaacaatttccaaggcaaaaggccagccaagggaaccccaggtgtacactctgcctccaagccgcgatgagctgacaaagaaccaggtgtccctgacttgtctggtcaaagggttctatccctccgacatcgccgtggagtgggaatctaatggacagcctgagaacaattacaagaccacaccccctgtgctggactcagatgggagcttctttctgtattctaagctgactgtggacaaaagtagatggcagcagggaaacgtgttttcttgcagtgtcatgcacgaggccctgcacaatcattacacccagaagtcactgagcctgtccccaggaggaggaggaggaagcgagcccaagagctccgataaaactcatacctgcccaccctgtcctgctccagaactgctgggaggccctagcgtgttcctgtttcctccaaagccaaaagacacactgatgatttctaggactcccgaggtgacctgcgtggtggtcgatgtcagtcacgaggaccctgaagtgaagttcaactggtacgtggatggagtcgaggtgcacaacgccaagaccaaaccccgggaggaacagtacaacagcacctatagagtggtctccgtgctgacagtcctgcaccaggactggctgaacgggaaggaatacaagtgcaaagtgtccaataaggccctgcccgctcctatcgaaaaaaccatttctaaggctaaaggccagccccgggagccacaggtgtacacactgcccccttctcgggatgaactgaccaagaaccaggtgagtctgacatgtctggtcaaaggcttctatccaagtgacatcgcagtggagtgggaatcaaatgggcagcccgagaacaattacaagactaccccacccgtgctggactccgatggctctttctttctgtattcaaagctgaccgtggacaaaagcagatggcagcaggggaacgtgttcagctgcagtgtcatgcacgaagcactgcacaatcattacactcagaaatcactgtcactgtcacctgga

SEQ ID NO: 50 - DNA, 2505 bp, Artificial Sequence
  Sequence nucleique codant scFcL-GPAD2 (SEQ ID NO37)
gataagacacacacttgcccaccctgccctgcccctgagctgctgggcgggccaagcgtgttcctgtttccccctaagccaaaagatacactgatgatcagtagaactcccgaagtgacctgcgtggtcgtggacgtctcacacgaggaccccgaagtgaagttcaactggtacgtggacggcgtggaggtccataatgccaagaccaaaccccgcgaggaacagtacaactccacctatcgagtcgtgtctgtgctgacagtcctgcaccaggattggctgaacggcaaggagtataagtgcaaagtgtctaataaggctctgccagcacccatcgagaaaaccattagtaaggcaaaagggcagcctagggaaccacaggtgtacacactgccacccagtcgcgatgagctgactaagaaccaggtgtcactgacctgtctggtcaaaggattctatccttcagacatcgccgtggagtgggaaagcaatggccagccagagaacaattacaagaccacacctccagtgctggactctgatggaagtttctttctgtatagcaagctgactgtggacaaatccagatggcagcagggcaacgtgttttcttgcagtgtcatgcacgaggccctgcacaatcattacacccagaagtcactgagcctgtcccctggaggaggaggaggcagtggaggaggagggtcaggaggcgggggaagcgagccaaagagctccgataaaacacatacttgccccccttgtcctgctccagaactgctgggaggaccttccgtgttcctgtttccacccaagcctaaagacacactgatgatttccaggacaccagaagtgacttgtgtcgtggtcgacgtgtctcatgaggaccccgaggtgaagtttaactggtacgtggatggagtcgaagtgcacaacgccaagaccaaaccccgggaggaacagtacaatagtacttatagagtggtctcagtgctgaccgtcctgcaccaggactggctgaatggcaaggaatataagtgcaaagtgagcaataaggccctgcccgctcctatcgaaaaaactatttccaaggctaaaggccagccccgagagcctcaggtgtacaccctgcctccaagccgggatgaactgacaaagaaccaggtgtccctgacttgtctggtcaaagggttctatccctccgacatcgcagtggagtgggaatctaatggacagcctgagaacaattacaagactaccccccctgtgctggactcagatgggagcttctttctgtattctaagctgaccgtggataaaagtcgctggcagcagggaaatgtgttttcttgtagtgtcatgcacgaagccctgcataaccactatactcaaaagtcactgagcctgagcccaggaggaggaggaggctccaacaagtataaagacggggatcagtgcgagacatctccttgtcagaatcagggaaagtgtaaagacggcctgggggagtacacctgcacatgtctggagggcttcgaagggaagaactgcgaactgtttacaagaaaactgtgtagcctggataacggcgactgcgatcagttctgtcatgaggaacagaattccgtggtctgctcttgtgccaggggctacaccctggctgacaatgggaaggcatgcatccctaccgggccatatccctgtggaaaacagacactggagcggagaaagaggcgcaaacggatcgtggggggacaggagtgcaaggatggcgaatgtccatggcaggctctgctgattaacgaggaaaatgaggggttctgcggcgggactatcctgtccgaattttacattctgaccgccgctcactgtctgtatcaggctaagcggttcaaggtgcgggtcggcgacagaaacaccgagcaggaggaaggaggcgaagcagtgcacgaggtcgaagtggtcatcaagcataatcgcttcactaaagagacctacgactttgatatcgccgtgctgagactgaagacacccattactttcaggatgaacgtggcaccagcatgcctgccagagcgagattgggctgaatctaccctgatgacacagaaaactggcattgtgagtggatttggccgaacacatgagaaggggaggcagtctactcgcctgaaaatgctggaagtgccctacgtcgaccggaactcctgtaagctgtctagttcattcatcatcacacagaacatgttttgcgccggatatgacactaagcaggaagatgcttgtcagggcgacagcggaggacctcacgtgaccagattcaaagatacctattttgtgacaggcatcgtctcctggggggagggatgcgcaaggaagggaaaatacggcatctataccaaggtgacagccttcctgaaatggattgaccgatcaatgaagacacggggcctgcccaaggcaaaaagccatgcccctgaagtgatcactagctccccactgaaa

SEQ ID NO: 51 - DNA, 2496 bp, Artificial Sequence
  Sequence nucleique codant scFcL-GPAD1 (SEQ ID NO38)
gataagacacacacttgcccaccctgccctgcccctgagctgctgggcgggccaagcgtgttcctgtttccccctaagccaaaagatacactgatgatcagtagaactcccgaagtgacctgcgtggtcgtggacgtctcacacgaggaccccgaagtgaagttcaactggtacgtggacggcgtggaggtccataatgccaagaccaaaccccgcgaggaacagtacaactccacctatcgagtcgtgtctgtgctgacagtcctgcaccaggattggctgaacggcaaggagtataagtgcaaagtgtctaataaggctctgccagcacccatcgagaaaaccattagtaaggcaaaagggcagcctagggaaccacaggtgtacacactgccacccagtcgcgatgagctgactaagaaccaggtgtcactgacctgtctggtcaaaggattctatccttcagacatcgccgtggagtgggaaagcaatggccagccagagaacaattacaagaccacacctccagtgctggactctgatggaagtttctttctgtatagcaagctgactgtggacaaatccagatggcagcagggcaacgtgttttcttgcagtgtcatgcacgaggccctgcacaatcattacacccagaagtcactgagcctgtcccctggaggaggaggaggcagtggaggaggagggtcaggaggcgggggaagcgagccaaagagctccgataaaacacatacttgccccccttgtcctgctccagaactgctgggaggaccttccgtgttcctgtttccacccaagcctaaagacacactgatgatttccaggacaccagaagtgacttgtgtcgtggtcgacgtgtctcatgaggaccccgaggtgaagtttaactggtacgtggatggagtcgaagtgcacaacgccaagaccaaaccccgggaggaacagtacaatagtacttatagagtggtctcagtgctgaccgtcctgcaccaggactggctgaatggcaaggaatataagtgcaaagtgagcaataaggccctgcccgctcctatcgaaaaaactatttccaaggctaaaggccagccccgagagcctcaggtgtacaccctgcctccaagccgggatgaactgacaaagaaccaggtgtccctgacttgtctggtcaaagggttctatccctccgacatcgcagtggagtgggaatctaatggacagcctgagaacaattacaagactaccccccctgtgctggactcagatgggagcttctttctgtattctaagctgaccgtggataaaagtcgctggcagcagggaaatgtgttttcttgtagtgtcatgcacgaagccctgcataaccactatactcaaaagtcactgagcctgagcccaggaggaggaggaggctccaacaagtataaagacggggatcagtgcgagacatctccttgtcagaatcagggaaagtgtaaagacggcctgggggagtacacctgcacatgtctggagggcttcgaagggaagaactgcgaactgtttacaagaaaactgtgtagcctggataacggcgactgcgatcagttctgtcatgaggaacagaattccgtggtctgctcttgtgccaggggctacaccctggctgacaatgggaaggcatgcatccctaccgggccatatccctgtggaaaacagacactggagcggagaaagaggatcgtggggggacaggagtgcaaggatggcgaatgtccatggcaggctctgctgattaacgaggaaaatgaggggttctgcggcgggactatcctgtccgaattttacattctgaccgccgctcactgtctgtatcaggctaagcggttcaaggtgcgggtcggcgacagaaacaccgagcaggaggaaggaggcgaagcagtgcacgaggtcgaagtggtcatcaagcataatcgcttcactaaagagacctacgactttgatatcgccgtgctgagactgaagacacccattactttcaggatgaacgtggcaccagcatgcctgccagagcgagattgggctgaatctaccctgatgacacagaaaactggcattgtgagtggatttggccgaacacatgagaaggggaggcagtctactcgcctgaaaatgctggaagtgccctacgtcgaccggaactcctgtaagctgtctagttcattcatcatcacacagaacatgttttgcgccggatatgacactaagcaggaagatgcttgtcagggcgacagcggaggacctcacgtgaccagattcaaagatacctattttgtgacaggcatcgtctcctggggggagggatgcgcaaggaagggaaaatacggcatctataccaaggtgacagccttcctgaaatggattgaccgatcaatgaagacacggggcctgcccaaggcaaaaagccatgcccctgaagtgatcactagctccccactgaaa

SEQ ID NO: 52 - protein, 364 aa, Artificial Sequence
  Exemple de GPAD3
NKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERGGGGSRRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 53 - protein, 360 aa, Artificial Sequence; identical to SEQ ID NO: 28
  Exemple de GPAD3

SEQ ID NO: 54 - protein, 231 aa, Artificial Sequence
  SEQ ID NO39 comprenant la region charniere entiere en N-terminal
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 10 aa, Artificial Sequence
  Region charniere partielle N-terminal
DKTHTCPPCP

SEQ ID NO: 56 - protein, 15 aa, Artificial Sequence
  Region charniere entiere N-terminal
EPKSCDKTHTCPPCP

SEQ ID NO: 57 - DNA, 1080 bp, Artificial Sequence
  Sequence nucleique codant pour GPAD3-LC (SEQ ID NO28)
aacaagtacaaggacggggaccagtgtgagacctccccttgccagaaccagggcaagtgtaaagatggactgggcgagtacacctgcacatgtctggagggattcgaaggcaagaattgcgaactgtttactagaaaactgtgttctctggataacggggactgcgatcagttctgtcacgaggaacagaattccgtggtctgctcttgtgccagggggtacaccctggccgacaacggaaaggcttgcatccctaccggaccctatccttgtggcaaacagacactggagcgagggagctccggacgaagaaagcgaatcgtgggaggacaggagtgcaaagatggggaatgtccatggcaggccctgctgattaacgaggaaaatgagggattctgcggaggcactatcctgagcgaattttacattctgaccgccgctcattgtctgtatcaggccaagaggtttaaagtgcgcgtcggcgaccgaaacacagagcaggaggaagggggagaagctgtgcacgaggtcgaagtggtcatcaagcataatcgcttcactaaagagacctacgactttgatatcgcagtgctgagactgaagacaccaattactttcaggatgaatgtcgcaccagcatgcctgccagagcgagattgggctgaaagtaccctgatgacacagaagactggaattgtgtcagggtttggacggacacacgagaagggccggcagtctactagactgaaaatgctggaagtgccctacgtcgacaggaacagttgtaagctgtctagttcattcatcatcacacagaacatgttttgcgcaggctatgacactaagcaggaggatgcctgtcagggggacagcggaggacctcacgtgacccgcttcaaagatacttattttgtgaccggaatcgtcagctggggagagggatgcgctcgaaagggcaaatacgggatctataccaaggtgacagcattcctgaaatggattgacaggagcatgaagacccgcggcctgcctaaggctaaatcccatgcaccagaagtgatcacaagctcccccctgaag

SEQ ID NO: 58 - DNA, 1098 bp, Artificial Sequence
  Sequence nucleique codant pour GPAD3-LL (SEQ ID NO29)
aacaagtataaagacggagaccagtgcgagacatccccttgccagaaccagggcaagtgtaaagatgggctgggagagtacacctgcacatgtctggaggggttcgaaggaaagaattgcgaactgtttactagaaaactgtgttctctggataacggggactgcgatcagttctgtcacgaggaacagaattcagtggtctgcagctgtgccagggggtacaccctggccgacaacggaaaggcttgcatccccaccggaccctatccttgtggcaaacagacactggagagaggaggaggaggatccggaggaggagggtctcggagaaagaggatcgtgggaggccaggagtgcaaagatggggaatgtccttggcaggccctgctgattaacgaggaaaatgagggattctgcgggggaactatcctgagcgaattttacattctgaccgccgctcactgtctgtatcaggccaagaggtttaaagtgcgcgtcggcgaccgaaacacagagcaggaggaaggcggggaagctgtgcacgaggtcgaagtggtcatcaagcataatcgcttcactaaagagacctacgactttgatatcgcagtgctgcgactgaagacaccaattactttccggatgaatgtcgcaccagcatgcctgccagagcgagattgggctgaaagtaccctgatgacacagaagactggaattgtgtcaggctttgggcggacacatgagaagggccggcagtctactagactgaaaatgctggaagtgccttacgtcgacaggaacagttgtaagctgagctcctctttcatcattacacagaatatgttttgcgcaggctatgacactaagcaggaggatgcatgtcagggggacagcggaggaccacacgtgacccgcttcaaagatacttattttgtgaccggaatcgtcagctggggagagggatgcgctcgaaagggcaaatacgggatctataccaaggtgacagcattcctgaaatggattgacaggagcatgaagacccgcggcctgcctaaggctaaatcccatgcaccagaagtgatcacaagttcacccctgaag

SEQ ID NO: 59 - DNA, 1107 bp, Artificial Sequence
  Sequence nucleique codant pour GPAD3-2F (SEQ ID NO30)
aacaagtacaaggacggggatcagtgcgagacctccccttgccagaaccagggcaagtgtaaagatgggctgggagagtacacctgcacatgtctggaggggttcgaaggaaagaattgcgaactgtttactcgcaaactgtgttctctggataacggggactgcgatcagttctgtcacgaggaacagaattcagtggtctgcagctgtgccagaggctacaccctggccgacaacggaaaggcttgcatccccaccggaccctatccttgtggcaagcagacactggagcgaagaaaacgaggaggaggaggatccggaggaggagggtctaggcgcaagcgaatcgtgggaggccaggagtgcaaagatggggaatgtccttggcaggccctgctgattaacgaggaaaatgagggattctgcgggggaactatcctgagcgaattttacattctgaccgccgctcactgtctgtatcaggccaagcggttcaaggtgcgggtcggcgacagaaacacagagcaggaggaaggcggggaagctgtgcacgaggtcgaagtggtcatcaagcataatagattcactaaagagacctacgactttgatatcgcagtgctgagactgaagacaccaattactttcaggatgaatgtcgcaccagcctgcctgcccgagagagattgggctgaaagtaccctgatgacacagaaaactggaattgtgtcaggctttgggaggacacatgagaagggcaggcagtctactcgcctgaaaatgctggaagtgccttacgtcgacaggaacagttgtaagctgagctcctctttcatcattacacagaatatgttttgcgcaggctatgacactaagcaggaggatgcatgtcagggggacagcggaggaccacacgtgacccgcttcaaagatacttattttgtgaccggaatcgtcagctggggagagggatgcgctcgaaagggcaaatacgggatctataccaaggtgacagcattcctgaaatggattgaccgaagcatgaagacccggggcctgcctaaggctaaatcccatgcaccagaagtgatcacaagttcacccctgaag

SEQ ID NO: 60 - protein, 374 aa, Artificial Sequence
  GPAD1 avec peptide signal MB7
MRWSWIFLLLLSITSANASNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 61 - protein, 377 aa, Artificial Sequence
  GPAD2 avec peptide signal MB7
MRWSWIFLLLLSITSANASNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 62 - protein, 373 aa, Artificial Sequence
  GPAD1 optimise avec peptide signal MB7
MRWSWIFLLLLSITSANANKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 63 - protein, 376 aa, Artificial Sequence
  GPAD2 optimise avec peptide signal MB7
MRWSWIFLLLLSITSANANKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 64 - protein, 377 aa, Artificial Sequence
  SEQ ID NO27 avec peptide signal MB7
MRWSWIFLLLLSITSANANKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLEGSSGRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 65 - protein, 378 aa, Artificial Sequence
  GPAD3-LC avec peptide signal MB7
MRWSWIFLLLLSITSANANKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERGSSGRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 66 - protein, 384 aa, Artificial Sequence
  GPAD3-LL avec peptide signal MB7
MRWSWIFLLLLSITSANANKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERGGGGSGGGGSRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 67 - protein, 387 aa, Artificial Sequence
  GPAD3-2F avec peptide signal MB7
MRWSWIFLLLLSITSANANKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRGGGGSGGGGSRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 68 - protein, 604 aa, Artificial Sequence
  GPAD2-FX-Fc avec peptide signal MB7
MRWSWIFLLLLSITSANASNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 609 aa, Artificial Sequence
  GPAD2-Fcl avec peptide signal MB7
MRWSWIFLLLLSITSANASNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 608 aa, Artificial Sequence
  GPAD2-FcLss avec peptide signal MB7
MRWSWIFLLLLSITSANANKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 849 aa, Artificial Sequence
  GPAD2-scFcL avec peptide signal MB7
MRWSWIFLLLLSITSANASNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 839 aa, Artificial Sequence
  GPAD2-scFcS avec peptide signal MB7
MRWSWIFLLLLSITSANASNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 853 aa, Artificial Sequence
  scFcL-GPAD2 avec peptide signal MB7
MRWSWIFLLLLSITSANADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 74 - protein, 850 aa, Artificial Sequence
  scFcL-GPAD1 avec peptide signal MB7
MRWSWIFLLLLSITSANADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 75 - protein, 382 aa, Artificial Sequence
  GPAD 3 de SEQ ID NO52 avec peptide signal MB7
MRWSWIFLLLLSITSANANKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERGGGGSRRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 76 - protein, 378 aa, Artificial Sequence; identical to SEQ ID NO: 65
  GPAD3 de SEQ ID NO53 avec peptide signal MB7

SEQ ID NO: 77 - DNA, 1122 bp, Artificial Sequence
  Sequence nucleique codant pour la protine SEQ ID NO60
atgagatggagttggatttttctgctgctgctgagtattacctctgctaacgcaagcaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 78 - DNA, 1131 bp, Artificial Sequence
  Sequence nucleique codant pour la protine SEQ ID NO61
atgagatggagttggatttttctgctgctgctgagtattacctctgctaacgcaagcaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggaggaagaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 79 - DNA, 1119 bp, Artificial Sequence
  Sequence nucleique codant pour la proteine SEQ ID NO62
atgagatggagttggatttttctgctgctgctgagtattacctctgctaacgcaaacaagtataaagacggagaccagtgtgagactagcccttgccagaaccaggggaagtgtaaagatggactgggcgagtacacctgcacatgtctggagggattcgaaggcaagaattgcgaactgtttactagaaaactgtgtagcctggataacggcgactgcgatcagttctgtcacgaggaacagaattcagtggtctgcagctgtgccaggggatacaccctggccgacaacggcaaggcttgcatccctaccggcccctatccttgtgggaaacagacactggagcggagaaagaggatcgtgggcgggcaggagtgcaaggatggagaatgtccatggcaggccctgctgattaacgaggaaaatgagggcttctgcggaggcactatcctgtccgaattttacattctgaccgccgctcattgtctgtatcaggccaagcggttcaaggtgcgggtcggcgacagaaacacagagcaggaggaagggggagaagctgtgcacgaggtcgaagtggtcatcaagcataatcgcttcactaaagagacctacgactttgatatcgcagtgctgagactgaagacaccaattactttcaggatgaatgtcgcaccagcatgcctgccagagcgagattgggctgaatccaccctgatgacacagaaaactggcattgtgtctgggtttggacggacacacgagaaggggaggcagagcactcgcctgaaaatgctggaagtgccctacgtcgacaggaactcctgtaagctgagctcctctttcatcattacacagaatatgttttgcgcagggtatgacactaagcaggaggatgcctgtcagggagactctggaggacctcacgtgacccgcttcaaagatacttattttgtgaccggaatcgtcagttggggagagggatgcgctcgaaaggggaaatacggaatctataccaaggtgacagcattcctgaaatggattgaccgaagtatgaagacccggggcctgcctaaggctaaatcacatgcaccagaagtgatcacaagttcacccctgaag

SEQ ID NO: 80 - DNA, 1128 bp, Artificial Sequence
  Sequence nucleique codant pour la proteine SEQ ID NO63
atgagatggagttggatttttctgctgctgctgagtattacctctgctaacgcaaacaagtataaagacggagaccagtgtgagactagcccttgccagaaccaggggaagtgtaaagatggactgggcgagtacacctgcacatgtctggagggattcgaaggcaagaattgcgaactgtttactagaaaactgtgtagcctggataacggcgactgcgatcagttctgtcacgaggaacagaattcagtggtctgcagctgtgccaggggatacaccctggccgacaacggcaaggcttgcatccctaccggcccctatccttgtgggaaacagacactggagcggagaaagaggcgcaaacggatcgtgggcgggcaggagtgcaaggatggagaatgtccatggcaggccctgctgattaacgaggaaaatgagggcttctgcggaggcactatcctgtccgaattttacattctgaccgccgctcattgtctgtatcaggccaagcggttcaaggtgcgggtcggcgacagaaacacagagcaggaggaagggggagaagctgtgcacgaggtcgaagtggtcatcaagcataatcgcttcactaaagagacctacgactttgatatcgcagtgctgagactgaagacaccaattactttcaggatgaatgtcgcaccagcatgcctgccagagcgagattgggctgaatccaccctgatgacacagaaaactggcattgtgtctgggtttggacggacacacgagaaggggaggcagagcactcgcctgaaaatgctggaagtgccctacgtcgacaggaactcctgtaagctgagctcctctttcatcattacacagaatatgttttgcgcagggtatgacactaagcaggaggatgcctgtcagggagactctggaggacctcacgtgacccgcttcaaagatacttattttgtgaccggaatcgtcagttggggagagggatgcgctcgaaaggggaaatacggaatctataccaaggtgacagcattcctgaaatggattgaccgaagtatgaagacccggggcctgcctaaggctaaatcacatgcaccagaagtgatcacaagttcacccctgaag

SEQ ID NO: 81 - DNA, 1134 bp, Artificial Sequence
  Sequence nucleique codant pour la proteine SEQ ID NO65
atgcgatggagttggatctttctgctgctgctgagtattaccagtgcaaatgctaacaagtacaaggacggggaccagtgtgagacctccccttgccagaaccagggcaagtgtaaagatggactgggcgagtacacctgcacatgtctggagggattcgaaggcaagaattgcgaactgtttactagaaaactgtgttctctggataacggggactgcgatcagttctgtcacgaggaacagaattccgtggtctgctcttgtgccagggggtacaccctggccgacaacggaaaggcttgcatccctaccggaccctatccttgtggcaaacagacactggagcgagggagctccggacgaagaaagcgaatcgtgggaggacaggagtgcaaagatggggaatgtccatggcaggccctgctgattaacgaggaaaatgagggattctgcggaggcactatcctgagcgaattttacattctgaccgccgctcattgtctgtatcaggccaagaggtttaaagtgcgcgtcggcgaccgaaacacagagcaggaggaagggggagaagctgtgcacgaggtcgaagtggtcatcaagcataatcgcttcactaaagagacctacgactttgatatcgcagtgctgagactgaagacaccaattactttcaggatgaatgtcgcaccagcatgcctgccagagcgagattgggctgaaagtaccctgatgacacagaagactggaattgtgtcagggtttggacggacacacgagaagggccggcagtctactagactgaaaatgctggaagtgccctacgtcgacaggaacagttgtaagctgtctagttcattcatcatcacacagaacatgttttgcgcaggctatgacactaagcaggaggatgcctgtcagggggacagcggaggacctcacgtgacccgcttcaaagatacttattttgtgaccggaatcgtcagctggggagagggatgcgctcgaaagggcaaatacgggatctataccaaggtgacagcattcctgaaatggattgacaggagcatgaagacccgcggcctgcctaaggctaaatcccatgcaccagaagtgatcacaagctcccccctgaag

SEQ ID NO: 82 - DNA, 1152 bp, Artificial Sequence
  Sequence nucleique codant pour la proteine SEQ ID NO66
atgagatggagttggattttcctgctgctgctgagtatcaccagtgccaatgcaaacaagtataaagacggagaccagtgcgagacatccccttgccagaaccagggcaagtgtaaagatgggctgggagagtacacctgcacatgtctggaggggttcgaaggaaagaattgcgaactgtttactagaaaactgtgttctctggataacggggactgcgatcagttctgtcacgaggaacagaattcagtggtctgcagctgtgccagggggtacaccctggccgacaacggaaaggcttgcatccccaccggaccctatccttgtggcaaacagacactggagagaggaggaggaggatccggaggaggagggtctcggagaaagaggatcgtgggaggccaggagtgcaaagatggggaatgtccttggcaggccctgctgattaacgaggaaaatgagggattctgcgggggaactatcctgagcgaattttacattctgaccgccgctcactgtctgtatcaggccaagaggtttaaagtgcgcgtcggcgaccgaaacacagagcaggaggaaggcggggaagctgtgcacgaggtcgaagtggtcatcaagcataatcgcttcactaaagagacctacgactttgatatcgcagtgctgcgactgaagacaccaattactttccggatgaatgtcgcaccagcatgcctgccagagcgagattgggctgaaagtaccctgatgacacagaagactggaattgtgtcaggctttgggcggacacatgagaagggccggcagtctactagactgaaaatgctggaagtgccttacgtcgacaggaacagttgtaagctgagctcctctttcatcattacacagaatatgttttgcgcaggctatgacactaagcaggaggatgcatgtcagggggacagcggaggaccacacgtgacccgcttcaaagatacttattttgtgaccggaatcgtcagctggggagagggatgcgctcgaaagggcaaatacgggatctataccaaggtgacagcattcctgaaatggattgacaggagcatgaagacccgcggcctgcctaaggctaaatcccatgcaccagaagtgatcacaagttcacccctgaag

SEQ ID NO: 83 - DNA, 1161 bp, Artificial Sequence
  Sequence nucleique codant pour la proteine SEQ ID NO67
atgcgatggagctggatttttctgctgctgctgtcaattacatctgccaacgccaacaagtacaaggacggggatcagtgcgagacctccccttgccagaaccagggcaagtgtaaagatgggctgggagagtacacctgcacatgtctggaggggttcgaaggaaagaattgcgaactgtttactcgcaaactgtgttctctggataacggggactgcgatcagttctgtcacgaggaacagaattcagtggtctgcagctgtgccagaggctacaccctggccgacaacggaaaggcttgcatccccaccggaccctatccttgtggcaagcagacactggagcgaagaaaacgaggaggaggaggatccggaggaggagggtctaggcgcaagcgaatcgtgggaggccaggagtgcaaagatggggaatgtccttggcaggccctgctgattaacgaggaaaatgagggattctgcgggggaactatcctgagcgaattttacattctgaccgccgctcactgtctgtatcaggccaagcggttcaaggtgcgggtcggcgacagaaacacagagcaggaggaaggcggggaagctgtgcacgaggtcgaagtggtcatcaagcataatagattcactaaagagacctacgactttgatatcgcagtgctgagactgaagacaccaattactttcaggatgaatgtcgcaccagcctgcctgcccgagagagattgggctgaaagtaccctgatgacacagaaaactggaattgtgtcaggctttgggaggacacatgagaagggcaggcagtctactcgcctgaaaatgctggaagtgccttacgtcgacaggaacagttgtaagctgagctcctctttcatcattacacagaatatgttttgcgcaggctatgacactaagcaggaggatgcatgtcagggggacagcggaggaccacacgtgacccgcttcaaagatacttattttgtgaccggaatcgtcagctggggagagggatgcgctcgaaagggcaaatacgggatctataccaaggtgacagcattcctgaaatggattgaccgaagcatgaagacccggggcctgcctaaggctaaatcccatgcaccagaagtgatcacaagttcacccctgaag

SEQ ID NO: 84 - DNA, 1812 bp, Artificial Sequence
  Sequence nucleique codant pour la proteine SEQ ID NO68
atgagatggagttggatttttctgctgctgctgagtattacctctgctaacgcaagcaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggaggaagaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaaggacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 85 - DNA, 1827 bp, Artificial Sequence
  Sequence nucleique codant pour la proteine SEQ ID NO69
atgagatggagttggatttttctgctgctgctgagtattacctctgctaacgcaagcaataagtataaagacggggatcagtgcgagacctccccatgtcagaaccagggcaagtgcaaagacgggctgggagagtacacatgcacttgtctggaggggttcgaaggaaagaattgcgaactgtttacaagaaagctgtgcagcctggataacggcgactgcgatcagttctgtcacgaggaacagaatagtgtggtctgctcatgtgccagggggtacactctggctgacaacggaaaggcatgcatccctactggaccttatccatgtggcaaacagaccctggagcggagaaagaggcgcaaacgcatcgtgggcgggcaggagtgcaaggatggagaatgtccatggcaggctctgctgattaacgaggaaaatgagggcttctgcggaggcacaatcctgagcgagttctacattctgactgccgctcattgtctgtatcaggctaagcggttcaaggtgcgggtcggcgacagaaacaccgagcaggaggaagggggagaagcagtgcacgaggtcgaagtggtcatcaagcataatcgcttcacaaaagagacttacgactttgatatcgccgtgctgagactgaagacccccattacattcaggatgaatgtggcaccagcatgcctgcctgagcgagattgggctgaatcaactctgatgacccagaaaacaggaattgtgagcggctttgggcgaactcacgagaagggcaggcagagcacccgcctgaaaatgctggaagtgccctacgtcgaccggaacagctgtaagctgagctcctctttcatcattacccagaatatgttttgcgccggctatgacacaaagcaggaggatgcttgtcagggggactccggaggacctcatgtgaccagattcaaagatacatattttgtgactggcatcgtctcttggggagaaggctgcgccaggaagggcaaatacgggatctatactaaggtgaccgccttcctgaaatggattgatcgatccatgaagactcggggcctgccaaaggcaaaatctcacgcccccgaagtgatcaccagttcacctctgaaggaacctaagtcttgcgacaaaacccatacatgcccaccttgtccagcacctgaactgctgggaggaccatccgtgttcctgtttccacccaagcccaaagatacactgatgattagtcggacccctgaggtgacatgcgtggtcgtggatgtctcacacgaggacccagaagtgaagtttaactggtacgtggacggcgtggaagtccataatgccaagaccaaacctcgcgaggaacagtacaacagtacatatcgagtcgtgtcagtgctgactgtcctgcaccaggattggctgaacggaaaggagtataagtgcaaagtgagcaataaggctctgccagcacccatcgagaaaacaatttccaaggcaaaaggccagccaagggaaccccaggtgtacactctgcctccaagccgcgatgagctgacaaagaaccaggtgtccctgacttgtctggtcaaagggttctatccctccgacatcgccgtggagtgggaatctaatggacagcctgagaacaattacaagaccacaccccctgtgctggactcagatgggagcttctttctgtattctaagctgactgtggacaaaagtagatggcagcagggaaacgtgttttcttgcagtgtcatgcacgaggccctgcacaatcattacacccagaagtcactgagcctgtccccaggaaag

SEQ ID NO: 86 - DNA, 1824 bp, Artificial Sequence
  Sequence nucleique codant pour la proteine SEQ ID NO70
atgagatggagttggatttttctgctgctgctgagtattacctctgctaacgcaaataagtataaagacggggatcagtgcgagacctctccttgtcagaaccagggcaagtgcaaagacgggctgggagagtacacatgcacttgtctggaggggttcgaaggaaagaattgcgaactgtttacaagaaaactgtgtagcctggataacggggactgcgatcagttctgtcacgaggaacagaattccgtggtctgctcttgtgcaagggggtacaccctggctgacaacggaaaggcatgcatccctactggaccttatccatgtggcaaacagaccctggagcggagaaagaggcgcaaacgcatcgtgggcgggcaggagtgcaaggatggagaatgtccatggcaggctctgctgattaacgaggaaaatgagggcttctgcggaggcacaatcctgagcgaattttacattctgactgccgctcattgtctgtatcaggctaagcggttcaaggtgcgggtcggcgacagaaacaccgagcaggaggaagggggagaagcagtgcacgaggtcgaagtggtcatcaagcataatcgcttcacaaaagagacttacgactttgatatcgccgtgctgagactgaagacccccattacattcaggatgaatgtggcaccagcatgcctgcctgagcgagattgggctgaatctactctgatgacccagaaaacaggaattgtgagtggctttgggcgaactcacgagaagggcaggcagtctacccgcctgaaaatgctggaagtgccctacgtcgaccggaacagctgtaagctgagctcctctttcatcattacccagaatatgttttgcgccggctatgacacaaagcaggaggatgcttgtcagggggacagcggaggacctcatgtgaccagattcaaagatacatattttgtgactggcatcgtctcctggggagaaggctgcgcaaggaagggcaaatacgggatctatactaaggtgaccgccttcctgaaatggattgatcgatcaatgaagactcggggcctgccaaaggcaaaaagccacgcccccgaagtgatcaccagttcacctctgaaggaaccaaagagctgcgacaaaacccatacatgcccaccttgtccagcacctgaactgctgggaggaccatccgtgttcctgtttccacccaagcccaaagatacactgatgatttcccggacccctgaggtgacatgtgtggtcgtggatgtctctcacgaggacccagaagtgaagtttaactggtacgtggacggcgtggaagtccataatgccaagaccaaaccccgcgaggaacagtacaactccacatatcgagtcgtgtctgtgctgactgtcctgcaccaggattggctgaacggaaaagagtacaagtgcaaagtgagtaataaggctctgccagcacccatcgagaaaacaatttccaaggcaaaaggccagccaagggaaccccaggtgtacactctgcctccaagtcgcgatgagctgacaaagaaccaggtgtcactgacttgtctggtcaaagggttctatccctcagacatcgccgtggagtgggaaagcaatggacagcctgagaacaattacaagaccacaccccctgtgctggactctgatgggagtttctttctgtatagcaagctgactgtggacaaatccagatggcagcagggaaacgtgttttcttgcagtgtcatgcacgaggccctgcacaatcattacacccagaagtcactgagcctgtccccaggaaag

SEQ ID NO: 87 - DNA, 2547 bp, Artificial Sequence
  Sequence nucleique codant pour la proteine SEQ ID NO71
atgagatggagttggatttttctgctgctgctgagtattacctctgctaacgcaagcaataagtataaagacggggatcagtgcgagacctccccatgtcagaaccagggcaagtgcaaagacgggctgggagagtacacatgcacttgtctggaggggttcgaaggaaagaattgcgaactgtttacaagaaagctgtgcagcctggataacggcgactgcgatcagttctgtcacgaggaacagaatagtgtggtctgctcatgtgccagggggtacactctggctgacaacggaaaggcatgcatccctactggaccttatccatgtggcaaacagaccctggagcggagaaagaggcgcaaacgcatcgtgggcgggcaggagtgcaaggatggagaatgtccatggcaggctctgctgattaacgaggaaaatgagggcttctgcggaggcacaatcctgagcgagttctacattctgactgccgctcattgtctgtatcaggctaagcggttcaaggtgcgggtcggcgacagaaacaccgagcaggaggaagggggagaagcagtgcacgaggtcgaagtggtcatcaagcataatcgcttcacaaaagagacttacgactttgatatcgccgtgctgagactgaagacccccattacattcaggatgaatgtggcaccagcatgcctgcctgagcgagattgggctgaatcaactctgatgacccagaaaacaggaattgtgagcggctttgggcgaactcacgagaagggcaggcagagcacccgcctgaaaatgctggaagtgccctacgtcgaccggaacagctgtaagctgagctcctctttcatcattacccagaatatgttttgcgccggctatgacacaaagcaggaggatgcttgtcagggggactccggaggacctcatgtgaccagattcaaagatacatattttgtgactggcatcgtctcttggggagaaggctgcgccaggaagggcaaatacgggatctatactaaggtgaccgccttcctgaaatggattgatcgatccatgaagactcggggcctgccaaaggcaaaatctcacgcccccgaagtgatcaccagttcacctctgaaggacaaaacccatacatgcccaccttgtccagcacctgaactgctgggaggaccatccgtgttcctgtttccacccaagcccaaagatacactgatgattagtcggacccctgaggtgacatgcgtggtcgtggatgtctcacacgaggacccagaagtgaagtttaactggtacgtggacggcgtggaagtccataatgccaagaccaaacctcgcgaggaacagtacaacagtacatatcgagtcgtgtcagtgctgactgtcctgcaccaggattggctgaacggaaaggagtataagtgcaaagtgagcaataaggctctgccagcacccatcgagaaaacaatttccaaggcaaaaggccagccaagggaaccccaggtgtacactctgcctccaagccgcgatgagctgacaaagaaccaggtgtccctgacttgtctggtcaaagggttctatccctccgacatcgccgtggagtgggaatctaatggacagcctgagaacaattacaagaccacaccccctgtgctggactcagatgggagcttctttctgtattctaagctgactgtggacaaaagtagatggcagcagggaaacgtgttttcttgcagtgtcatgcacgaggccctgcacaatcattacacccagaagtcactgagcctgtccccaggaggaggaggaggaagcggaggaggaggctccgggggaggcgggtctgagcccaagagctccgataaaactcatacctgcccaccctgtcctgctccagaactgctgggaggccctagcgtgttcctgtttcctccaaagccaaaagacacactgatgatttctaggactcccgaggtgacctgcgtggtggtcgatgtcagtcacgaggaccctgaagtgaagttcaactggtacgtggatggagtcgaggtgcacaacgccaagaccaaaccccgggaggaacagtacaacagcacctatagagtggtctccgtgctgacagtcctgcaccaggactggctgaacgggaaggaatacaagtgcaaagtgtccaataaggccctgcccgctcctatcgaaaaaaccatttctaaggctaaaggccagccccgggagccacaggtgtacacactgcccccttctcgggatgaactgaccaagaaccaggtgagtctgacatgtctggtcaaaggcttctatccaagtgacatcgcagtggagtgggaatcaaatgggcagcccgagaacaattacaagactaccccacccgtgctggactccgatggctctttctttctgtattcaaagctgaccgtggacaaaagcagatggcagcaggggaacgtgttcagctgcagtgtcatgcacgaagcactgcacaatcattacactcagaaatcactgtcactgtcacctgga

SEQ ID NO: 88 - DNA, 2517 bp, Artificial Sequence
  Sequence nucleique codant pour la proteine SEQ ID NO72
atgagatggagttggatttttctgctgctgctgagtattacctctgctaacgcaagcaataagtataaagacggggatcagtgcgagacctccccatgtcagaaccagggcaagtgcaaagacgggctgggagagtacacatgcacttgtctggaggggttcgaaggaaagaattgcgaactgtttacaagaaagctgtgcagcctggataacggcgactgcgatcagttctgtcacgaggaacagaatagtgtggtctgctcatgtgccagggggtacactctggctgacaacggaaaggcatgcatccctactggaccttatccatgtggcaaacagaccctggagcggagaaagaggcgcaaacgcatcgtgggcgggcaggagtgcaaggatggagaatgtccatggcaggctctgctgattaacgaggaaaatgagggcttctgcggaggcacaatcctgagcgagttctacattctgactgccgctcattgtctgtatcaggctaagcggttcaaggtgcgggtcggcgacagaaacaccgagcaggaggaagggggagaagcagtgcacgaggtcgaagtggtcatcaagcataatcgcttcacaaaagagacttacgactttgatatcgccgtgctgagactgaagacccccattacattcaggatgaatgtggcaccagcatgcctgcctgagcgagattgggctgaatcaactctgatgacccagaaaacaggaattgtgagcggctttgggcgaactcacgagaagggcaggcagagcacccgcctgaaaatgctggaagtgccctacgtcgaccggaacagctgtaagctgagctcctctttcatcattacccagaatatgttttgcgccggctatgacacaaagcaggaggatgcttgtcagggggactccggaggacctcatgtgaccagattcaaagatacatattttgtgactggcatcgtctcttggggagaaggctgcgccaggaagggcaaatacgggatctatactaaggtgaccgccttcctgaaatggattgatcgatccatgaagactcggggcctgccaaaggcaaaatctcacgcccccgaagtgatcaccagttcacctctgaaggacaaaacccatacatgcccaccttgtccagcacctgaactgctgggaggaccatccgtgttcctgtttccacccaagcccaaagatacactgatgattagtcggacccctgaggtgacatgcgtggtcgtggatgtctcacacgaggacccagaagtgaagtttaactggtacgtggacggcgtggaagtccataatgccaagaccaaacctcgcgaggaacagtacaacagtacatatcgagtcgtgtcagtgctgactgtcctgcaccaggattggctgaacggaaaggagtataagtgcaaagtgagcaataaggctctgccagcacccatcgagaaaacaatttccaaggcaaaaggccagccaagggaaccccaggtgtacactctgcctccaagccgcgatgagctgacaaagaaccaggtgtccctgacttgtctggtcaaagggttctatccctccgacatcgccgtggagtgggaatctaatggacagcctgagaacaattacaagaccacaccccctgtgctggactcagatgggagcttctttctgtattctaagctgactgtggacaaaagtagatggcagcagggaaacgtgttttcttgcagtgtcatgcacgaggccctgcacaatcattacacccagaagtcactgagcctgtccccaggaggaggaggaggaagcgagcccaagagctccgataaaactcatacctgcccaccctgtcctgctccagaactgctgggaggccctagcgtgttcctgtttcctccaaagccaaaagacacactgatgatttctaggactcccgaggtgacctgcgtggtggtcgatgtcagtcacgaggaccctgaagtgaagttcaactggtacgtggatggagtcgaggtgcacaacgccaagaccaaaccccgggaggaacagtacaacagcacctatagagtggtctccgtgctgacagtcctgcaccaggactggctgaacgggaaggaatacaagtgcaaagtgtccaataaggccctgcccgctcctatcgaaaaaaccatttctaaggctaaaggccagccccgggagccacaggtgtacacactgcccccttctcgggatgaactgaccaagaaccaggtgagtctgacatgtctggtcaaaggcttctatccaagtgacatcgcagtggagtgggaatcaaatgggcagcccgagaacaattacaagactaccccacccgtgctggactccgatggctctttctttctgtattcaaagctgaccgtggacaaaagcagatggcagcaggggaacgtgttcagctgcagtgtcatgcacgaagcactgcacaatcattacactcagaaatcactgtcactgtcacctgga

SEQ ID NO: 89 - DNA, 2559 bp, Artificial Sequence
  Sequence nucleique codant pour la proteine SEQ ID NO73
atgcgatggtcctggatttttctgctgctgctgagtattacctctgccaacgccgataagacacacacttgcccaccctgccctgcccctgagctgctgggcgggccaagcgtgttcctgtttccccctaagccaaaagatacactgatgatcagtagaactcccgaagtgacctgcgtggtcgtggacgtctcacacgaggaccccgaagtgaagttcaactggtacgtggacggcgtggaggtccataatgccaagaccaaaccccgcgaggaacagtacaactccacctatcgagtcgtgtctgtgctgacagtcctgcaccaggattggctgaacggcaaggagtataagtgcaaagtgtctaataaggctctgccagcacccatcgagaaaaccattagtaaggcaaaagggcagcctagggaaccacaggtgtacacactgccacccagtcgcgatgagctgactaagaaccaggtgtcactgacctgtctggtcaaaggattctatccttcagacatcgccgtggagtgggaaagcaatggccagccagagaacaattacaagaccacacctccagtgctggactctgatggaagtttctttctgtatagcaagctgactgtggacaaatccagatggcagcagggcaacgtgttttcttgcagtgtcatgcacgaggccctgcacaatcattacacccagaagtcactgagcctgtcccctggaggaggaggaggcagtggaggaggagggtcaggaggcgggggaagcgagccaaagagctccgataaaacacatacttgccccccttgtcctgctccagaactgctgggaggaccttccgtgttcctgtttccacccaagcctaaagacacactgatgatttccaggacaccagaagtgacttgtgtcgtggtcgacgtgtctcatgaggaccccgaggtgaagtttaactggtacgtggatggagtcgaagtgcacaacgccaagaccaaaccccgggaggaacagtacaatagtacttatagagtggtctcagtgctgaccgtcctgcaccaggactggctgaatggcaaggaatataagtgcaaagtgagcaataaggccctgcccgctcctatcgaaaaaactatttccaaggctaaaggccagccccgagagcctcaggtgtacaccctgcctccaagccgggatgaactgacaaagaaccaggtgtccctgacttgtctggtcaaagggttctatccctccgacatcgcagtggagtgggaatctaatggacagcctgagaacaattacaagactaccccccctgtgctggactcagatgggagcttctttctgtattctaagctgaccgtggataaaagtcgctggcagcagggaaatgtgttttcttgtagtgtcatgcacgaagccctgcataaccactatactcaaaagtcactgagcctgagcccaggaggaggaggaggctccaacaagtataaagacggggatcagtgcgagacatctccttgtcagaatcagggaaagtgtaaagacggcctgggggagtacacctgcacatgtctggagggcttcgaagggaagaactgcgaactgtttacaagaaaactgtgtagcctggataacggcgactgcgatcagttctgtcatgaggaacagaattccgtggtctgctcttgtgccaggggctacaccctggctgacaatgggaaggcatgcatccctaccgggccatatccctgtggaaaacagacactggagcggagaaagaggcgcaaacggatcgtggggggacaggagtgcaaggatggcgaatgtccatggcaggctctgctgattaacgaggaaaatgaggggttctgcggcgggactatcctgtccgaattttacattctgaccgccgctcactgtctgtatcaggctaagcggttcaaggtgcgggtcggcgacagaaacaccgagcaggaggaaggaggcgaagcagtgcacgaggtcgaagtggtcatcaagcataatcgcttcactaaagagacctacgactttgatatcgccgtgctgagactgaagacacccattactttcaggatgaacgtggcaccagcatgcctgccagagcgagattgggctgaatctaccctgatgacacagaaaactggcattgtgagtggatttggccgaacacatgagaaggggaggcagtctactcgcctgaaaatgctggaagtgccctacgtcgaccggaactcctgtaagctgtctagttcattcatcatcacacagaacatgttttgcgccggatatgacactaagcaggaagatgcttgtcagggcgacagcggaggacctcacgtgaccagattcaaagatacctattttgtgacaggcatcgtctcctggggggagggatgcgcaaggaagggaaaatacggcatctataccaaggtgacagccttcctgaaatggattgaccgatcaatgaagacacggggcctgcccaaggcaaaaagccatgcccctgaagtgatcactagctccccactgaaa

SEQ ID NO: 90 - DNA, 2550 bp, Artificial Sequence
  Sequence nucleique codant pour la proteine SEQ ID NO74
atgcgatggtcctggatttttctgctgctgctgagtattacctctgccaacgccgataagacacacacttgcccaccctgccctgcccctgagctgctgggcgggccaagcgtgttcctgtttccccctaagccaaaagatacactgatgatcagtagaactcccgaagtgacctgcgtggtcgtggacgtctcacacgaggaccccgaagtgaagttcaactggtacgtggacggcgtggaggtccataatgccaagaccaaaccccgcgaggaacagtacaactccacctatcgagtcgtgtctgtgctgacagtcctgcaccaggattggctgaacggcaaggagtataagtgcaaagtgtctaataaggctctgccagcacccatcgagaaaaccattagtaaggcaaaagggcagcctagggaaccacaggtgtacacactgccacccagtcgcgatgagctgactaagaaccaggtgtcactgacctgtctggtcaaaggattctatccttcagacatcgccgtggagtgggaaagcaatggccagccagagaacaattacaagaccacacctccagtgctggactctgatggaagtttctttctgtatagcaagctgactgtggacaaatccagatggcagcagggcaacgtgttttcttgcagtgtcatgcacgaggccctgcacaatcattacacccagaagtcactgagcctgtcccctggaggaggaggaggcagtggaggaggagggtcaggaggcgggggaagcgagccaaagagctccgataaaacacatacttgccccccttgtcctgctccagaactgctgggaggaccttccgtgttcctgtttccacccaagcctaaagacacactgatgatttccaggacaccagaagtgacttgtgtcgtggtcgacgtgtctcatgaggaccccgaggtgaagtttaactggtacgtggatggagtcgaagtgcacaacgccaagaccaaaccccgggaggaacagtacaatagtacttatagagtggtctcagtgctgaccgtcctgcaccaggactggctgaatggcaaggaatataagtgcaaagtgagcaataaggccctgcccgctcctatcgaaaaaactatttccaaggctaaaggccagccccgagagcctcaggtgtacaccctgcctccaagccgggatgaactgacaaagaaccaggtgtccctgacttgtctggtcaaagggttctatccctccgacatcgcagtggagtgggaatctaatggacagcctgagaacaattacaagactaccccccctgtgctggactcagatgggagcttctttctgtattctaagctgaccgtggataaaagtcgctggcagcagggaaatgtgttttcttgtagtgtcatgcacgaagccctgcataaccactatactcaaaagtcactgagcctgagcccaggaggaggaggaggctccaacaagtataaagacggggatcagtgcgagacatctccttgtcagaatcagggaaagtgtaaagacggcctgggggagtacacctgcacatgtctggagggcttcgaagggaagaactgcgaactgtttacaagaaaactgtgtagcctggataacggcgactgcgatcagttctgtcatgaggaacagaattccgtggtctgctcttgtgccaggggctacaccctggctgacaatgggaaggcatgcatccctaccgggccatatccctgtggaaaacagacactggagcggagaaagaggatcgtggggggacaggagtgcaaggatggcgaatgtccatggcaggctctgctgattaacgaggaaaatgaggggttctgcggcgggactatcctgtccgaattttacattctgaccgccgctcactgtctgtatcaggctaagcggttcaaggtgcgggtcggcgacagaaacaccgagcaggaggaaggaggcgaagcagtgcacgaggtcgaagtggtcatcaagcataatcgcttcactaaagagacctacgactttgatatcgccgtgctgagactgaagacacccattactttcaggatgaacgtggcaccagcatgcctgccagagcgagattgggctgaatctaccctgatgacacagaaaactggcattgtgagtggatttggccgaacacatgagaaggggaggcagtctactcgcctgaaaatgctggaagtgccctacgtcgaccggaactcctgtaagctgtctagttcattcatcatcacacagaacatgttttgcgccggatatgacactaagcaggaagatgcttgtcagggcgacagcggaggacctcacgtgaccagattcaaagatacctattttgtgacaggcatcgtctcctggggggagggatgcgcaaggaagggaaaatacggcatctataccaaggtgacagccttcctgaaatggattgaccgatcaatgaagacacggggcctgcccaaggcaaaaagccatgcccctgaagtgatcactagctccccactgaaa

SEQ ID NO: 91 - protein, 11 aa, Artificial Sequence
  Linker de l′exemple 20
GGGGSGGGGSR

SEQ ID NO: 92 - protein, 8 aa, Artificial Sequence
  Sequence decrite a la figure 9
RKRIVGGQ

SEQ ID NO: 93 - protein, 12 aa, Artificial Sequence
  Sequence decrite a l′exemple 9
RRKRRKRIVGGQ

(19) United States
(12) Patent Application Publication
(10) Pub. No.: US 2016/0177286 A1
(43) Pub. Date: Jun. 23, 2016

(54) GLA-DOMAINLESS FACTOR X

(71) Applicants: LABORATOIRE FRANCAIS DU FRACTIONNEMENT ET DES BIOTECHNOLOGIES, Les Ulis (FR); CENTRE NATIONAL DE LA RECHERCHE SCIENTIFIQUE - CNRS, Paris Cedex 16 (FR); UNIVERSITE GRENOBLE I JOSEPH FOURIER, Saint Martin D'heres (FR)

(72) Inventors: Aline Thomas, Meylan (FR); Marie-Claire Dagher, Fontaine (FR); Raphael Marlu, La Tronche (FR); Benoit Polack, Saint Martin (FR); Jean-Luc Plantier, Croix (FR); Alexandre Fontayne, La Madeleine (FR); Toufik Abache, Santes (FR)

(73) Assignees: LABORATOIRE FRANCAIS DU FRACTIONNEMENT ET DES BIOTECHNOLOGIES, Les Ulis (FR); CENTRE NATIONAL DE LA RECHERCHE SCIENTIFIQUE -CNRS, Paris Cedex 16 (FR); UNIVERSITE GRENOBLE I JOSEPH FOURIER, Saint Martin D'heres (FR)

(21) Appl. No.: 14/898,994

(22) PCT Filed: Jun. 20, 2014
(86) PCT No.: PCT/FR2014/051542

§ 371 Date: Mar. 10, 2016

(30) Foreign Application Priority Data
Jun. 21, 2013 (FR) 13 55896

(51) Int. Cl. C12N 9/64

(52) U.S. Cl. CPC C12N 9/6432; C07K 2319/30; C07K 2319/33; C12Y 304/21006; A61K 38/00

(57) ABSTRACT

The present invention relates to a protein consisting of the sequence SEQ ID No.: 11 or 25 or 26, directly bound, or bound via a linker, especially -Arg-Lys-Arg-, to the sequence SEQ ID No.: 6.

The present invention relates to factor X mutants, and to the use thereof for treating blood coagulation disorders.

Factor X is a protein present in the blood. This protein plays an important role in the coagulation cascade. Blood coagulation is a complex process which makes it possible to prevent blood flow via damaged vessels. As soon as a vessel is broken, the elements responsible for coagulation interact with one another to form a plug, the hemostatic plug, at the site where the vessel is broken. The coagulation factors are required in order to hold the hemostatic plug in place and to stabilize the clot.

The formation of a normal clot occurs in four steps:

Step 1 The blood vessel is damaged.
Step 2 The blood vessel contracts so as to restrict the blood supply to the damaged zone.
Step 3 The platelets adhere to the subendothial space exposed during the damaging of the vessel and also to the stimulated blood vessel walls. The platelets spread, this is what is referred to as “platelet adhesion”. These spread platelets release substances which activate other neighboring platelets such that they agglomerate at the seat of the lesion in order to form the hemostatic plug. This is what is referred to as “platelet aggregation”.
Step 4 The surface of the activated platelets thus constitutes a surface on which blood coagulation can take place. The coagulation proteins which circulate in the blood (including factor X) are activated at the surface of platelets and form a fibrin clot.

These coagulation proteins (i.e. factors I, II, V, VIII, IX, X, XI, XII and XIII, and also Von Willebrand factor) operate in a chain reaction, i.e. the coagulation cascade.

Factor X in activated form (Xa) is involved more particularly in the activation of prothrombin (factor II) to thrombin (factor IIa), in particular when it is complexed with activated cofactor V so as to form the prothrombinase complex. This factor is an essential element in the coagulation cascade.

When this factor is lacking, bleeding occurs, such as epistaxis (nose bleeds), hemarthrosis (effusion of blood into a joint cavity) or gastrointestinal bleeding. Factor X deficiency is extremely rare. Its transmission is autosomal recessive, and its prevalence is 1/1 000 000.

### FX Activation (FXa) Occurs:

either very early during the step of initiation of the coagulation cascade by the factor VIIa/tissue factor complex in a relatively ineffective reaction which results in the formation of traces of thrombin;
or during the step of amplification of the coagulation cascade resulting from positive feedback produced by the traces of thrombin, resulting in the activation of factors VIII and IX.

FXa forms the prothrombinase complex, which catalyzes the conversion of prothrombin to thrombin. Thrombin, for its part, catalyzes the conversion of fibrinogen to fibrin, which results in the formation of clots in the blood and in the arrest of bleeding. The activity of FXa can be referred to as “procoagulating activity”.

The two factors VIII and IX are missing in individuals suffering from hemophilia A and B, thus causing a hemorrhagic disorder which can be fatal without treatment. Hemophilia A, like hemophilia B, groups together two types of hemophilia, constitutional hemophilia and acquired hemophilia.

Constitutional hemophilia type A is a hemorrhagic disease characterized by a quantitative or qualitative deficiency in FVIII resulting from an abnormality in the FVIII gene. Constitutional hemophilia type B is also a hemorrhagic disease, but characterized by a quantitative or qualitative deficiency in FIX resulting from an abnormality in the FIX gene.

Acquired hemophilia type A or B is defined by the appearance of autoantibodies directed against this FVIII or this FIX.

Hemophilia results in a deficiency in blood coagulation in response to a hemorrhage. The absence of factors VIII and IX means that it is not possible to generate sufficient amounts of activated factor X to stop the hemorrhage.

Patients suffering from hemophilia A and B can be treated with concentrates comprising, respectively, FVIII or FIX which may be plasma derivatives or products resulting from genetic engineering. These concentrates can be administered when each hemorrhage occurs; in this case, it is advisable to begin the treatment as rapidly as possible, when the first signs appear. The treatment can also be administered prophylactically, regularly 2 to 3 times a week so as to prevent hemorrhages. However, the treatment can give rise to the appearance of antibodies directed against FVIII or FIX, called inhibitors. The presence of such antibodies then renders administrations of factor VIII or IX ineffective. These antibodies develop early as soon as the first administrations, often before the tenth. Some patients remain weak responders (antibody titer<5 Bethesda Units (BU)), others, called strong responders, achieve titers which no longer make it possible to treat them with the corresponding factor.

To date, there is no treatment which makes it possible to satisfactorily prevent and/or treat the existence of a hemorrhagic risk in patients suffering from hemophilia A or B and exhibiting an inhibitor. Indeed, the products available may be ineffective (Astermark J, Donfield S M, DiMichele D M, Gringeri A, Gilbert S A, Waters J, Berntorp E, for the FSG. A randomized comparison of bypassing agents in hemophilia complicated by an inhibitor: the FEIBA NovoSeven Comparative (FENOC) Study. Blood. 2007; 109: 546-51) or their administration may be complicated by thrombotic events (Aledort L M. Comparative thrombotic event incidence after infusion of recombinant factor VIIa versus factor VIII inhibitor bypass activity. J Thromb Haemost. 2004; 2: 1709).

There is therefore an established need for therapeutic alternatives to the existing treatments. Such alternatives must also have the following advantages:
they must make it possible to stop the hemorrhage,
they must not cause thrombosis, and
they must allow the treatment and/or prevention of hemorrhagic events even in the presence of anti-FVIII or anti-FIX antibodies.

The present invention meets this need. A subject of the present invention is a modified factor X (called GPAD-FXa) consisting of the sequence SEQ ID No.: 11 or SEQ ID No.: 25 or SEQ ID No.: 26, directly fused, or fused via a linker, in particular -Arg-Lys-Arg-, to the sequence SEQ ID No.: 6. A subject of the present invention is also a modified factor X (FX), said modified FX (called GPAD-FXa) consisting of the sequence SEQ ID No.: 11 directly fused, or fused via a linker, in particular -Arg-Lys-Arg-, to the sequence SEQ ID No.: 6.

Such a modified factor FXa is in particular of use for the prevention and/or treatment of a hemorrhagic event in a patient suffering from hemophilia A or B.

The present invention therefore relates to a protein consisting of the sequence SEQ ID No.: 11 or SEQ ID No.: 25 or SEQ ID No.: 26, directly fused, or fused via a linker, in particular -Arg-Lys-Arg-, to the sequence SEQ ID No.: 6. Such a protein is also called GPAD-FXa in the present application.

Examples of FX proteins according to the invention are in particular the sequences SEQ ID Nos.: 7 and 9, 21 to 24 and 60 to 63.

Another subject of the invention is a polynucleotide encoding said protein.

Another subject of the invention is an expression vector comprising said polynucleotide.

Another subject of the invention is a host cell comprising said expression vector or said polynucleotide.

Another subject of the invention is the use of said protein as a medicament. In particular, said protein can be used for the treatment of blood coagulation disorders, in particular hemorrhagic disorders, such as hemophilia A, B and C (factor XI deficiency), factor X deficiencies, or even emergency coagulation needs for replacing factor VIIa. When a powerful and rapid procoagulant response is required, said protein can be used in combination with other hemostatic molecules, such as factor VIIa and/or fibrinogen, or even in combination with procoagulant compounds (platelet transfusion, procoagulant mixture such as FEIBA, Kaskadil, Kanokad, etc.), which will be able to reinforce the efficacy of the treatment.

As used herein, the terms “protein” and “polypeptide” are used herein interchangeably and refer to an amino acid sequence having more than 100 amino acids.

The present invention relates to a mutant factor X consisting of the sequence SEQ ID No.: 11 or SEQ ID No.: 25 or SEQ ID No.: 26, directly fused, or fused via a linker, in particular -Arg-Lys-Arg-, to the sequence SEQ ID No.: 6. Preferably, the present invention relates to a mutant factor X consisting of the sequence SEQ ID No.: 26 directly fused, or fused via a linker -Arg-Lys-Arg-, to the sequence SEQ ID No.: 6. Preferably, the present invention relates to a mutant factor X consisting of the sequence SEQ ID No.: 11 directly fused, or fused via a linker -Arg-Lys-Arg-, to the sequence SEQ ID No.: 6. Preferably, the GPAD-FXa according to the invention is single-stranded.

Factor X, also called Stuart-Prower factor, is encoded by the F10 gene and refers to the serine protease EC3.4.21.6. Factor X is composed of a heavy chain of 306 amino acids and of a light chain of 139 amino acids.

Factor X is a protein of 488 amino acids, consisting of a signal peptide, a propeptide, and light and heavy chains.

The sequence of human factor X can be found in UniProtKB under accession number P00742.

The protein is translated in the form of a prepropeptide. After cleavage of the signal peptide, the propeptide is finally cleaved, and the factor X is cleaved into a light chain and a heavy chain (respectively of 142 and 306 amino acids) (zymogen), which remain associated by non-covalent interactions and/or a disulfide bridge. In this double-stranded form, factor X is inactive. Following the triggering of coagulation, the heavy chain is finally activated by cleavage of the activation peptide, so as to contain only 254 amino acids (the first 52 amino acids are cleaved during the processing): this is the heavy chain of factor Xa (SEQ ID No.: 6).

The prepropeptide of human factor X corresponds to SEQ ID No.: 4. The heavy chain with activation peptide corresponds to SEQ ID No.: 1, and the light chain corresponds to SEQ ID No.: 5. The activation peptide of the heavy chain corresponds to SEQ ID No.: 3, and comprises 52 amino acids.

SEQ ID No.: 2 (signal peptide and light chain) is identical to amino acids 1 to 182 of

SEQ ID No.: 4.

SEQ ID No.: 1 (heavy chain with activation peptide) is identical to amino acids 183 to 488 of SEQ ID No.: 4.

The heavy chain of factor Xa (SEQ ID No.: 6) corresponds to SEQ ID No.: 1, in which the peptide SEQ ID No.: 3 has been cleaved.

According to one particular embodiment, a subject of the invention is a modified factor X (called GPAD-FXa) consisting of the sequence SEQ ID No.: 11 or SEQ ID No.: 25 or SEQ ID No.: 26, directly fused, or fused via a linker, in particular -Arg-Lys-Arg-, to the sequence SEQ ID No.: 6, itself being directly fused, or fused via a linker, at the C-terminal end of SEQ ID No.: 6, to a tag sequence comprising from 1 to 25 amino acids, preferably from 1 to 20 amino acids. Preferably, the tag sequence is the HPC4 tag of sequence SEQ ID No.: 20, or the tag of sequence SEQ ID No.: 20 from which the cysteine amino acid in position 1 has been deleted. Preferentially, the linker placed at the C-terminal of SEQ ID No.: 6 is chosen from the sequences -GSSG-, -(GGGGS)n-, n being between 1 and 5, and -GSSGSSG-. Advantageously, the tag sequence allows visualization of the GPAD-FXa protein according to the invention in vitro and in vivo, and/or allows the protein of interest to be assayed and/or purified.

In this embodiment, the nucleic sequences encoding the GPAD-FXa protein according to the invention are fused, at their C-terminal end, to a nucleic sequence encoding a tag sequence, directly or via a nucleic sequence encoding an abovementioned linker.

The modified factor X according to the invention, GPAD-FXa, lacks its phospholipid-binding γ-carboxyglutamic acid (Gla) domain and its activation peptide (SEQ ID No.: 3).

The first 42 amino acids of the light chain (residues 1-42 of SEQ ID No.: 5) represent the Gla domain, since it contains 11 post-translationally modified residues (γ-carboxyglutamic acid). Digestion with chymotrypsin makes it possible to delete residues 1-42 making it possible to enzymatically generate an FX lacking its phospholipid-binding domain. Alternatively, this molecule may also be expressed directly without this domain by genetic engineering. GPAD-FXa thus contains only a fragment of the light chain, i.e. the sequence SEQ ID No.: 11 or the sequence SEQ ID No.: 25 or the sequence SEQ ID No.: 26. The sequences SEQ ID No.: 11, SEQ ID No.: 25 and SEQ ID No.: 26 differ only by the optional addition of one or two amino acids in the N-terminal position. GPAD-FXa is also:
either fused directly to the sequence SEQ ID No.: 6, in particular so as to give the mutant of sequence SEQ ID No.: 9;
or fused via a linker, in particular -Arg-Lys-Arg-, to the sequence SEQ ID No.: 6, in particular so as to give the mutant of sequence SEQ ID No.: 7.

Preferably, the modified factor X according to the invention consists of the fragment of the light chain of sequence SEQ ID No.: 11 or SEQ ID No.: 25 or SEQ ID No.: 26 fused directly to the sequence SEQ ID No.: 6, which is itself optionally fused to a tag sequence. The modified factor X consisting of:
the fragment of the light chain of sequence SEQ ID No.: 11 fused directly to the sequence SEQ ID No.: 6, or
the fragment of the light chain of sequence SEQ ID No.: 25 fused directly to the sequence SEQ ID No.: 6 (this total sequence corresponds to the mutant of sequence SEQ ID No.: 21), or
the fragment of the light chain sequence SEQ ID No.: 26 fused directly to the sequence SEQ ID No.: 6,
   corresponds to the mutant called GPAD1.

Preferably, the modified factor X according to the invention consists of the fragment of the light chain of sequence SEQ ID No.: 25 fused via a linker, in particular -Arg-Lys-Arg-, to the sequence SEQ ID No.: 6, which is itself optionally fused to a tag sequence. The modified factor X consisting of:
the fragment of the light chain of sequence SEQ ID No.: 25 fused via a linker -Arg-Lys-Arg- to the sequence SEQ ID No.: 6 (this sequence corresponds to the mutant of sequence SEQ ID No.: 22), or
the fragment of the light chain of sequence SEQ ID No.: 11 fused via a linker -Arg-Lys-Arg- to the sequence SEQ ID No.: 6, or
the fragment of the light chain of sequence SEQ ID No.: 26 fused via a linker -Arg-Lys-Arg- to the sequence SEQ ID No.: 6,
   corresponds to the mutant called GPAD2.

Preferably, the modified factor X according to the invention is optimized for its expression by the host cell. The host cell may be a recombinant cell or a cell of a transgenic animal. In this case, it is preferably chosen from the sequences SEQ ID No.: 23 (called optimized GPAD1) and SEQ ID No.: 24 (called optimized GPAD2).

Such a GPAD-FXa may also comprise other modifications. In particular, it may comprise at least one mutation chosen from a point substitution, a deletion and an insertion, preferably a point substitution or an insertion. Thus, various mutations have been introduced into the gene encoding GPAD-FXa, making it possible to keep a thrombin-generating activity. These mutations can be introduced using the QuickChange kit (Stratagene) and by following the manufacturer's recommendations and according to the publication Wang & Malcolm (1999)—BioTechniques, 26: 680-682. These mutations can relate to arginine 138 of the heavy chain of GPAD-FXa (i.e. SEQ ID No.: 6), which can be mutated to give any other amino acid, preferentially phenylalanine (for example SEQ ID No.: 16), glycine (for example SEQ ID No.: 13), isoleucine (for example SEQ ID No.: 14) or tyrosine (for example SEQ ID No.: 15). Similarly, lysine 82 of the heavy chain (i.e. SEQ ID No.: 6) can also be replaced with an amino acid such as tyrosine (for example SEQ ID No.: 12). The GPAD-FXa according to the invention can thus comprise at least one of the mutations described above. It can also comprise the double mutation of arginine 138 of SEQ ID No.: 6 to an amino acid chosen from phenylalanine, glycine, isoleucine and tyrosine, and of lysine 82 of SEQ ID No.: 6 to tyrosine. Preferably, it comprises the double mutation of arginine 138 of SEQ ID No.: 6 to phenylalanine, and of lysine 82 of SEQ ID No.: 6 to tyrosine. Preferably, it comprises the double mutation of arginine 138 of SEQ ID No.: 6 to glycine, and of lysine 82 of SEQ ID No.: 6 to tyrosine. Preferably, it comprises the double mutation of arginine 138 of SEQ ID No.: 6 to isoleucine, and of lysine 82 of SEQ ID No.: 6 to tyrosine. Preferably, it comprises the double mutation of arginine 138 of SEQ ID No.: 6 to tyrosine, and of lysine 82 of SEQ ID No.: 6 to tyrosine.

Preferably, the GPAD-FXa according to the invention may also be modified by an insertion into the sequence SEQ ID No.: 11, 25 or 26.

Preferably, the insertion into the sequence SEQ ID No.: 11, 25 or 26 corresponds to the insertion of a linker. Preferably, the linker is chosen from the linkers:

-GSSG-, -RGSSG-, -GSSGR-, -RKRGSSGR-, -(GGGGS)n-, -R(GGGGS)n-, -(GGGGS)nR-, -RKR(GGGGS)nR-, in which n is an integer from 1 to 5, preferably 1 to 3, and
X, R—X, X—R and RKR—X—R, X being a peptide of 4 to 52 amino acids, G a glycine, S a serine, R an arginine and K a lysine.
X is preferably chosen from the following sequences (originating from the parts.igem.org web site):

| | | Length |
|---|---|---|
| | | (number |
| Accession | | of amino |
| code | Description | acids) |
| BBa_J18920 | 2aa GS linker | 6 |
| BBa_J18921 | 6aa [GS]x linker | 18 |
| BBa_J18922 | 10aa [GS]x linker | 30 |
| BBa_K105012 | 10 aa flexible protein domain linker | 30 |
| BBa_K133132 | 8 aa protein domain linker | 24 |
| BBa_K157009 | Split fluorophore linker; Freiburg standard | 51 |
| BBa_K157013 | 15 aa flexible glycine-serine protein domain | 45 |
| | linker; Freiburg standard | |
| BBa_K243004 | Short Linker (Gly-Gly-Ser-Gly) | 12 |
| BBa_K243005 | Middle Linker (Gly-Gly-Ser-Gly)x2 | 24 |
| BBa_K243006 | Long Linker (Gly-Gly-Ser-Gly)x3 | 36 |
| BBa_K416001 | (Gly4Ser)3 Flexible Peptide Linker | 45 |
| BBa_K648005 | Short Fusion Protein Linker: GGSG with | 12 |
| | standard 25 prefix/suffix | |
| BBa_K648006 | Long 10AA Fusion Protein Linker with | 30 |
| | Standard 25 Prefix/Suffix | |
| BBa_K648007 | Medium 6AA Fusion Protein Linker: | 18 |
| | GGSGGS with Standard 25 Prefix/Suffix | |

Preferably, the sequence SEQ ID No.: 25 is modified so as to comprise, between amino acids 99 and 100, an insertion of the linker -GSSGR-, -RKRGSSGR-, -(GGGGS)nR- or -RKR(GGGGS)nR-, in which n is an integer from 1 to 5, preferably from 1 to 3, X—R or RKR—X—R where X is a peptide of 4 to 52 amino acids, G a glycine, S a serine, R an arginine and K a lysine.

Alternatively, the sequence SEQ ID No.: 25 is preferably modified so as to comprise, between amino acids 98 and 99, an insertion of a linker. Preferably, the linker is a linker -GSSG-, -RGSSG-, -R(GGGGS)n- or -(GGGGS)n-, in which n is an integer from 1 to 5, preferably from 1 to 3, X or R—X where X is a peptide of 4 to 52 amino acids and G a glycine, S a serine and R an arginine.

Similarly, the sequence SEQ ID No.: 26 is preferably modified so as to comprise, between amino acids 98 and 99, an insertion of the linker -GSSGR-, -RKRGSSGR-, -(GGGGS)nR- or -RKR(GGGGS)nR-, in which n is an integer from 1 to 5, preferably from 1 to 3, or X—R or RKR—X—R where X is a peptide of 4 to 52 amino acids, G a glycine, S a serine, R an arginine and K a lysine.

Alternatively, the sequence SEQ ID No.: 26 is preferably modified so as to comprise, between amino acids 97 and 98, an insertion of the linker -GSSG-, -RGSSG-, -R(GGGGS)n- or -(GGGGS)n-, in which n is an integer from 1 to 5, preferably from 1 to 3, X or R—X where X is a peptide of 4 to 52 amino acids and G a glycine, S a serine and R an arginine.

Preferentially, according to a first alternative, the GPAD-FXa according to the invention consists of the fragment of the light chain of sequence SEQ ID No.: 26 fused directly to the sequence SEQ ID No.: 6, which is itself optionally directly fused to a tag sequence, in which the sequence SEQ ID No.: 26 is modified so as to comprise, between amino acids 98 and 99, an insertion of the linker -GSSGR-, -RKRGSSGR-, -(GGGGS)nR- or -RKR(GGGGS)nR-, in which n is an integer from 1 to 5, preferably from 1 to 3, or X—R or RKR—X—R where X is a peptide of 4 to 52 amino acids, G a glycine, S a serine, R an arginine and K a lysine.

Preferentially, according to a second alternative, the GPAD-FXa according to the invention consists of the fragment of the light chain of sequence SEQ ID No.: 26 fused to the sequence SEQ ID No.: 6, which is itself optionally fused to a tag sequence, in which the sequence SEQ ID No.: 26 is modified so as to comprise, between amino acids 97 and 98, an insertion of the linker -GSSG-, -RGSSG-, -R(GGGGS)n- or -(GGGGS)n-, in which n is an integer from 1 to 5, preferably from 1 to 3, X or R—X where X is a peptide of 4 to 52 amino acids and G a glycine, S a serine and R an arginine.

Such insertions correspond to the mutants called GPAD3.

Preferably, the GPAD-FXa according to the invention is chosen from the sequences SEQ ID No.: 28 (called GPAD3-LC), SEQ ID No.: 29 (called GPAD3-LL) and SEQ ID No.: 30 (called GPAD3-2F), SEQ ID No.: 27, SEQ ID No.: 52, SEQ ID NO:53, SEQ ID No.: 64, SEQ ID No.: 65, SEQ ID No.: 66, SEQ ID No.: 67, SEQ ID No.: 75 and SEQ ID No.: 76.

The GPAD3 as defined above can in particular be mutated on arginine 138 of the heavy chain (i.e. of the sequence SEQ ID No.: 6), which can be substituted to phenylalanine, to glycine, to isoleucine or to tyrosine. Similarly, the GPAD3 as defined above can be mutated on lysine 82 of the heavy chain (i.e. of the sequence SEQ ID No.: 6), which can be replaced with tyrosine.

According to one particular aspect of the invention, the GPAD3 can be mutated on arginine 138 of the heavy chain (i.e. of the sequence SEQ ID No.: 6) and also mutated on lysine 82 of the heavy chain (i.e. of the sequence SEQ ID No.: 6).

The GPAD-FXa according to the invention can also be fused, in the N-terminal or C-terminal position, to at least one wild-type immunoglobulin fragment. The term “wild-type immunoglobulin fragment” is intended to mean a fragment chosen from wild-type Fc fragments and wild-type scFc fragments.

The term “Fc fragment” is intended to mean the constant region of an immunoglobulin of complete length with the exclusion of the first immunoglobulin constant region domain (i.e. CH1-CL). Thus, the Fc fragment refers to a homodimer, each monomer comprising the last two IgA, IgD and IgG constant domains (i.e. CH2 and CH3), or the last three IgE and IgM constant domains (i.e. CH2, CH3 and CH4), and the N-terminal flexible hinge region of these domains. The Fc fragment, when it is derived from IgA or from IgM, may comprise the J chain. Preferably, the Fc region of an IgG1 is composed of the N-terminal flexible hinge and of the CH2-CH3 domains, i.e. the portion starting from the amino acid C226 up to the C-terminal end, the numbering being indicated according to the EU index or equivalent in Kabat.

The term “scFc fragment” (“single chain Fc”) is intended to mean a single chain Fc fragment obtained by genetic fusion of two Fc monomers linked by a polypeptide linker. The scFc folds naturally to give a functional dimeric Fc region.

The fusion of GPAD-FXa to at least one wild-type immunoglobulin fragment (in particular an Fc or scFc fragment) in the N-terminal or C-terminal position makes it possible to improve the stability and the retention of GPAD-FXa in the organism, and thus its bioavailability; it also makes it possible to improve its half-life in the organism. In addition, it may make it possible to simplify the purification of the molecule obtained by targeting the Fc fragment during one of the purification steps. Preferably, the wild-type Fc fragment is chosen from the sequence SEQ ID No.: 39 and the sequence SEQ ID No.: 54, optionally followed by a lysine in the C-terminal position (226 or 227 amino acids respectively for SEQ ID No.: 39; 231 or 232 amino acids respectively for SEQ ID No.: 54). The Fc fragment corresponding to the sequence SEQ ID No.: 39 comprises the CH2 and CH3 constant domains of a wild-type IgG and the partial hinge region in the N-terminal position (DKTHTCPPCP, SEQ ID No.: 55). The fragment corresponding to the sequence SEQ ID No.: 54 comprises the CH2 and CH3 constant domains of a wild-type IgG and the whole hinge region in the N-terminal position (sequence EPKSCDKTHTCPPCP, SEQ ID No.: 56).

Preferentially, GPAD2 is used so as to be fused in the C-terminal position with the Fc SEQ ID No.: 39, followed by lysine. Preferably, in this case, the GPAD-FXa fused to an Fc according to the invention corresponds to the sequence SEQ ID No.: 32 (GPAD2-FX-Fc). Alternatively, GPAD2 is used so as to be fused in the C-terminal position with the Fc SEQ ID No.: 54, followed by lysine. Preferably, in this case, the GPAD-FXa fused to an Fc according to the invention corresponds to the sequence SEQ ID No.: 33 (GPAD2-Fcl). Preferentially, GPAD2 can also be directly fused in the C-terminal position with the Fc SEQ ID No.: 39 or the Fc SEQ ID No.: 54, said Fc being itself fused in the C-terminal position to another identical or different Fc, via a linker -(GGGGS)n-, where n is an integer from 1 to 3, corresponding in this case to an scFc fragment. Preferably, in this case, the GPAD-FXa fused to an scFc according to the invention is chosen from SEQ ID No.: 35 (GPAD2-scFcL) and 36 (GPAD2-scFcS). The GPAD-FXa fused to an Fc according to the invention may also consist of the sequence SEQ ID No.: 26 fused via a linker -Arg-Lys-Arg- to the sequence SEQ ID No.: 6, itself fused to the Fc SEQ ID No.: 54 followed by a lysine. In this case, it has the sequence SEQ ID No.: 34 (GPAD2-FcLss).

Alternatively, GPAD2 or GPAD1 can be used so as to fused in the N-terminal position, directly or via a linker, with the FC SEQ ID No.: 39.

Preferentially, in this case, GPAD2 or GPAD1 is fused in the N-terminal position, via a linker -GGGGS-, with the Fc SEQ ID No.: 39 or the Fc SEQ ID No.: 54, said Fc being itself fused in the N-terminal position to another identical or different Fc, via a linker -(GGGGS)n-, where n is an integer from 1 to 3. Preferably, in this case, the GPAD-FXa fused to an Fc according to the invention is chosen from SEQ ID No.: 37 (scFcL-GPAD2) and 38 (scFcL-GPAD1).

According to the invention, in its immature form, GPAD-FXa corresponds to a single-stranded protein lacking an activation peptide. The cleavage between the heavy chain SEQ ID No.: 6 and the fragment of the light chain SEQ ID No.: 11 or SEQ ID No.: 25 or SEQ ID No.: 26 can either be carried out through the presence of the natural sequence for cleavage of factor X by cell enzymes, furins (RRKR), in the sequence SEQ ID No.: 11 or 25 or 26, will be carried out via a linker which makes it possible to improve the cleavage of the protein (i) in the producer cell; (ii) outside the cell during the molecule production/purification process; and/or (iii) during the activation of the molecule in vivo. The term “linker” is intended to mean a short sequence of amino acids, i.e. between 2 and 5 amino acids.

Among the possible linkers, those cleaved in the producer cell can, without limitations and by way of example, be those of the furin RX(K or R)R (Hosaka J Biol Chem 1991), or else of any other protease involved in intracellular protein maturation or activation. The linker may also be chosen from the cleavage sites of proteases involved in the coagulation cascade, such as, for example, activated protein C, kallikrein, FXIIa, FXIa, FXa, FIXa or FVIIa, The linker may also be composed of a peptide sequence known by those skilled in the art for separating recombinant proteins, such as, for example, the one recognized by TEV (Tobacco Etch Virus) or enterokinase. Preferably, the linker is a sequence of 3 amino acids. More preferentially, the linker is -Arg-Lys-Arg-. Advantageously, following cleavage of GPAD-FXa between the light chain and the heavy chain, GPAD-FXa is directly obtained in activated form, and does not therefore require any additional step of cleavage of the activation peptide.

Said protein according to the invention is a mutated factor X which is effective in the treatment of coagulation disorders.

The composition according to the invention can also be used for the prevention or treatment of a hemorrhagic event in hemophilic patients who exhibit anti-factor VIII (FVIII) or anti-factor IX (FIX) antibodies. The antibodies appeared either following a treatment with FVIII or FIX factors, or spontaneously, as in acquired hemophilia.

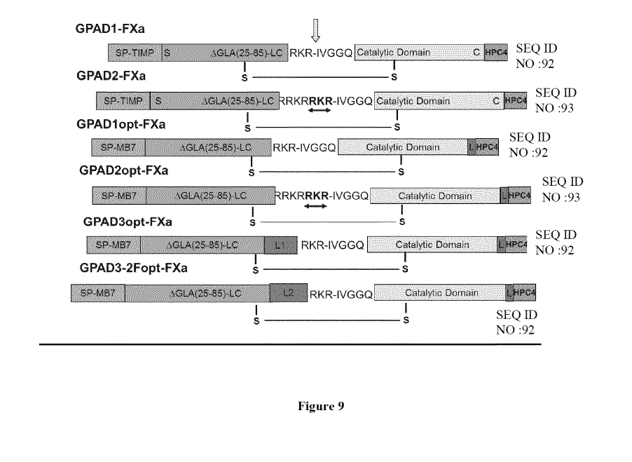

The sequences described in the present application can be summarized as follows:

| SEQ ID No.: | Protein |
|---|---|
| 1 | Heavy chain of human factor X (306 amino acids), |
| | comprising the activation peptide |
| 2 | Signal peptide and light chain of human factor X (182 |
| | amino acids) |
| 3 | Activation peptide of the heavy chain (52 amino acids) |
| 4 | Prepropeptide of human factor X (488 amino acids) |
| 5 | Light chain of human factor X (142 amino acids) |
| 6 | Heavy chain of activated human factor X (FXa) (254 |
| | amino acids) |
| 7 | Human factor X mutant GPAD-FXa according to the |
| | invention (357 amino acids) |
| 8 | Nucleic sequence encoding the mutant of SEQ ID No.: 7 |
| 9 | Human factor X mutant GPAD-FXa according to the |
| | invention (354 amino acids) |
| 10 | Nucleic sequence encoding the mutant of SEQ ID No.: 9 |
| 11 | Light chain fragment present in the sequences SEQ ID |
| | Nos.: 7 and 9 (100 amino acids) |
| 12 | Human factor X mutant GPAD-FXa identical to SEQ ID |
| | No.: 7 with K82Y |
| 13 | Human factor X mutant GPAD-FXa identical to SEQ ID |
| | No.: 7 with R138G |
| 14 | Human factor X mutant GPAD-FXa identical to SEQ ID |
| | No.: 7 with R138I |
| 15 | Human factor X mutant GPAD-FXa identical to SEQ ID |
| | No.: 7 with R138Y |
| 16 | Human factor X mutant GPAD-FXa identical to SEQ ID |
| | No.: 7 with R138F |
| 17-18 | Primers used in example |
| 19 | Signal peptide MB7 |
| 20 | HPC4 tag |
| 21 | GPAD1 |
| 22 | GPAD2 |
| 23 | Optimized GPAD1 |
| 24 | Optimized GPAD2 |
| 25 | SEQ ID No.: 11 also comprising the amino acids Ser- |
| | Asn-in the N-terminal position (i.e. 102 amino acids in |
| | total) |
| 26 | SEQ ID No.: 11 also comprising the amino acid Asn-in |
| | the N-terminal position (i.e. 101 amino acids in total) |
| 27 | Example of GPAD-3: SEQ ID No.: 26 comprising an |
| | insertion of -GSSG-between amino acids 97 and 98, |
| | directly fused to SEQ ID No.: 6 |
| 28 | GPAD3-LC |
| 29 | GPAD3-LL |
| 30 | GPAD3-2F |
| 31 | Heavy chain of activated human factor X (SEQ ID No.: 6) |
| | comprising, in the C-terminal position, a linker fused to a |
| | tag of sequence SEQ ID No.: 20 without the cysteine in |
| | position 1 |
| 32 | GPAD2-FX-Fc |
| 33 | GPAD2-Fcl |
| 34 | GPAD2-FcLss |
| 35 | GPAD2-scFcL |
| 36 | GPAD2-scFcS |
| 37 | scFcL-GPAD2 |
| 38 | scFcL-GPAD1 |
| 39 | Wild-type Fc fragment, optionally followed by a lysine |
| 40 to 51 | Nucleic sequences encoding respectively the sequences |
| | SEQ ID Nos.: 21 to 24 and 31 to 38 |
| 52 | Example of GPAD-3: SEQ ID No.: 26 comprising an |
| | insertion of -RGGGGS-between amino acids 97 and 98, |
| | fused via a linker Arg-Lys-Arg to SEQ ID No.: 6 |
| 53 | Example of GPAD-3: SEQ ID No.: 26 comprising an |
| | insertion of -GSSGR-between amino acids 98 and 99, |
| | directly fused to SEQ ID No.: 6 |
| 54 | SEQ ID No.: 39 (Wild-type Fc fragment) comprising the |
| | whole hinge region in the N-terminal position |
| 55 | N-terminal partial hinge region |
| 56 | N-terminal whole hinge region |
| 57 to 59 | Nucleic sequences encoding respectively the sequences |
| | SEQ ID Nos.: 28 to 30 |
| 60 | GPAD1 with signal peptide MB7 in the N-terminal |
| | position |
| 61 | GPAD2 with signal peptide MB7 in the N-terminal |
| | position |
| 62 | Optimized GPAD1 with signal peptide MB7 in the N- |
| | terminal position |
| 63 | Optimized GPAD2 with signal peptide MB7 in the N- |
| | terminal position |
| 64 | Example of GPAD-3 with signal peptide MB7 in the N- |
| | terminal position: SEQ ID No.: 27 with signal peptide |
| | MB7 in the N-terminal position |
| 65 | GPAD3-LC with signal peptide MB7 in the N-terminal |
| | position |
| 66 | GPAD3-LL with signal peptide MB7 in the N-terminal |
| | position |
| 67 | GPAD3-2F with signal peptide MB7 in the N-terminal |
| | position |
| 68 | GPAD2-FX-Fc with signal peptide MB7 in the N- |
| | terminal position |
| 69 | GPAD2-Fcl with signal peptide MB7 in the N-terminal |
| | position |
| 70 | GPAD2-FcLss with signal peptide MB7 in the N-terminal |
| | position |
| 71 | GPAD2-scFcL with signal peptide MB7 in the N-terminal |
| | position |
| 72 | GPAD2-scFcS with signal peptide MB7 in the N-terminal |
| | position |
| 73 | scFcL-GPAD2 with signal peptide MB7 in the N-terminal |
| | position |
| 74 | scFcL-GPAD1 with signal peptide MB7 in the N-terminal |
| | position |
| 75 | Example of GPAD-3: SEQ ID No.: 52 with signal peptide |
| | MB7 in the N-terminal position |
| 76 | Example of GPAD-3: SEQ ID No.: 53 with signal peptide |
| | MB7 in the N-terminal position |
| 77 to 90 | Nucleic sequences encoding respectively the sequences |
| | SEQ ID Nos.: 60 to 63 and 65 to 74 |
| 91 | Linker described in example 20 |
| 92 and 93 | Sequences described in FIG. 9 |

Another subject of the invention is a nucleic acid (polynucleotide or nucleotide sequence) encoding said protein. The nucleotide sequence encoding the GPAD-FXa can be synthesized chemically (Young L and Dong Q., 2004, -Nucleic Acids Res., April 1 5; 32(7), Hoover, D. M. and Lubkowski, J. 2002, Nucleic Acids Res., 30, Villalobos A, et al., 2006. BMC Bioinformatics, June 6; 7:285). The nucleotide sequence encoding the GPAD-FXa can also be amplified by PCR using suitable primers.

GPAD-FXa can also be produced by genetic engineering techniques well known to those skilled in the art. The nucleotide sequence encoding human factor X can thus be cloned into an expression vector; the part of the sequence encoding the signal peptide, the propeptide and the Gla domain is deleted, and a signal peptide is fused, for instance that of TIMP-1 (Crombez et al., 2005). The DNA encoding such a modified FX is inserted into an expression plasmid and inserted into a cell line ad hoc for its production (for example the FreeStyle HEK-293 line), the protein thus produced being subsequently purified by chromatography.

These techniques are described in detail in the reference manuals: Molecular cloning: a laboratory manual, 3rd edition-Sambrook and Russel eds. (2001) and Current Protocols in Molecular Biology—Ausubel et al. eds (2007).

The polynucleotide according to the invention is preferably chosen from the sequences SEQ ID No.: 8, SEQ ID No.: 10, SEQ ID Nos.: 40 to 51, SEQ ID Nos.: 57 to 59 and SEQ ID Nos.: 77 to 90.

The polynucleotide according to the invention encoding the GPAD-FXa may comprise, in the N-terminal position, a sequence encoding a signal peptide, which is in particular optimized. In one embodiment, the signal peptide used is that of TIMP-1. In one particularly advantageous embodiment, the signal peptide used is optimized for the expression and secretion of a GPAD-FXa protein according to the invention. Such signal peptides are described in particular in application WO 2011/114063. Preferably, a signal peptide used is MB7 (SEQ ID No.: 19: MRWSWIFLLLLSITSANA).

The polynucleotide according to the invention encoding the GPAD-FXa may also comprise optimized codons, optimized in particular for its expression in certain cells. For example, said cells comprise COS cells, CHO cells, HEK cells, BHK cells, PER. C6 cells, HeLa cells NIH/3T3 cells, 293 cells (ATCC # CRL1573 T2 cells, dendritic cells or monocytes. The objective of the codon optimization is to replace the natural codons with codons for which the transfer RNAs (tRNAs) carrying amino acids are the most frequent in the cell type under consideration. Mobilizing frequently encountered tRNAs has the major advantage of increasing the rate of translation of the messenger RNAs (mRNAs) and therefore of increasing the final titer (Carton J M et al, Protein Expr Purif 2007). The codon optimization also exploits the prediction of the mRNA secondary structures which might slow down the reading by the ribosomal complex. The codon optimization also has an impact on the G/C percentage which is directly linked to the half-life of mRNAs and therefore to their translation potential (Chechetkin, J. of Theoretical Biology 242, 2006 922-934).

The codon optimization can be carried out by substitution of the natural codons using codon usage tables for mammals and more particularly for Homo sapiens. There are algorithms present on the Internet and made available by the suppliers of synthetic genes (DNA2.0, GeneArt, MWG, Genscript) which make it possible to carry out this sequence optimization.

Preferably, the polynucleotide according to the invention comprises codons optimized for its expression in HEK cells, such as HEK293 cells. Such a polynucleotide is preferably chosen from the sequences SEQ ID No.: 42 and SEQ ID No.: 43. Alternatively, the polynucleotide according to the invention comprises codons optimized for its expression in the cells of transgenic animals, preferably goat, doe rabbit, ewe or cow.

The polynucleotide according to the invention may also advantageously be chosen from the sequences SEQ ID Nos.: 45 to 51.

Another subject of the invention is an expression cassette comprising said polynucleotide encoding said protein, or an expression vector comprising said polynucleotide or said expression cassette. According to the invention, the expression vectors appropriate for use according to the invention may comprise at least one expression control element functionally linked to the nucleic acid sequence. The expression control elements are inserted into the vector and make it possible to regulate the expression of the nucleic acid sequence. Examples of expression control elements include in particular lac systems, the lambda phage promoter, yeast promoters or viral promoters. Other operational elements may be incorporated, such as a leader sequence, stop codons, polyadenylation signals and sequences required for the transcription and subsequent translation of the nucleic acid sequence in the host system. It will be understood by those skilled in the art that the correct combination of expression control elements depends on the host system chosen. It will also be understood that the expression vector must contain the additional elements required for the transfer and subsequent replication of the expression vector containing the nucleic acid sequence in the host system.

Such vectors are easily constructed using conventional methods or are commercially available. Preferably, such vectors are those described in applications WO 2013/061010 and WO 2013/117871.

Another subject of the invention is a recombinant cell comprising an expression vector as described above, or a polynucleotide as described above. According to the invention, examples of host cells which can be used are eukaryotic cells, such as animal, vegetable, insect and yeast cells; and prokaryotic cells, such as E. coli. The means by which the vector carrying the gene can be introduced into the cells comprise in particular microinjection, electroporation, transduction or transfection using DEAE-dextran, lipofection, calcium phosphate or other procedures known to those skilled in the art. In one preferred embodiment, the eukaryotic expression vectors which function in eukaryotic cells are used. Examples of such vectors comprise viral vectors such as retroviruses, adenoviruses, herpes viruses, vaccinia virus, smallpox virus, poliovirus or lentiviruses, bacterial expression vectors or plasmids such as pcDNA5. The preferred eukaryotic cell lines comprise COS cells, CHO cells, HEK cells, BHK cells, Per.C6 cells, HeLa cells, NIH/3T3 cells, 293 cells (ATCC # CRL1573), T2 cells, dendritic cells or monocytes.

The protein according to the invention can be produced in the milk of transgenic animals.

In this case, according to a first aspect, the expression of a DNA sequence encoding the GPAD-FXa according to the invention is controlled by a mammalian casein promoter or a mammalian whey promoter, said promoter not naturally controlling the transcription of said gene, and the DNA sequence also containing a sequence for secretion of the protein. The secretion sequence comprises a secretion signal inserted between the coding sequence and the promoter. According to a particularly advantageous aspect, the DNA sequence encoding the GPAD-FXa comprises codons optimized for its expression in the cells of transgenic animals.

The transgenic animal used is capable not only of producing the desired protein, but also of transmitting this capacity to its progeny. The secretion of the protein into the milk facilitates purification and avoids the use of blood products. The animal can thus be chosen from goat, doe rabbit, ewe or cow.

Such a production process is in particular described in patent EP 0 264 166. This process may comprise the following steps:

(a) inserting into a non-human mammalian embryo a DNA sequence comprising a polynucleotide according to the invention, said polynucleotide being under the transcriptional control of a mammalian casein promoter or a mammalian whey promoter, said DNA sequence also comprising a signal sequence allowing the secretion of the GPAD-FXa protein,
(b) leaving said embryo to develop in an adult mammal,
(c) inducing lactation in said mammal or in a female descendant of said mammal in which said polynucleotide, the promoter and the signal sequence are present in the genome of the mammalian tissue,
(d) collecting the milk of said lactating mammal, and
(e) isolating said protein from said collected milk.

Preferably, a sequence encoding a furin enzyme or another specific endopeptidase is inserted into the non-human mammalian embryo in step a).

Preferably, the process for producing the GPAD-FXa according to the invention comprises the following steps:

(a) inserting into a non-human mammalian embryo a DNA sequence comprising a polynucleotide chosen from SEQ ID No.: 8, SEQ ID No.: 10, SEQ ID Nos.: 45 to 51, SEQ ID Nos.: 57 to 59 and SEQ ID Nos.: 77 to 90, said polynucleotide being under the transcriptional control of a mammalian casein promoter or a mammalian whey promoter, said DNA sequence also comprising a signal sequence allowing the secretion of said protein,
(b) leaving said embryo to develop in an adult mammal,
(c) inducing lactation in said mammal or in a female descendent of said mammal in which said polynucleotide, the promoter and the signal sequence are present in the genome of the mammalian tissue,
(d) collecting the milk of said lactating mammal, and
(e) isolating said protein from said collected milk.

Such a process directly uses a polynucleotide which does not contain sequence encoding an activation peptide.

Preferably, a sequence encoding a furin enzyme or another specific endopeptidase is inserted into the non-human mammalian embryo in step a).

The protein according to the invention can also be produced according to the following process:
a) transfecting eukaryotic cells for example HEK293 or CHO cells, with expression vectors comprising at least one polynucleotide according to the invention. The polynucleotide is preferably chosen from SEQ ID No.: 8, SEQ ID No.: 10, SEQ ID Nos.: 45 to 51, SEQ ID Nos.: 57 to 59 and SEQ ID Nos.: 77 to 90. Preferably, the cells are also transfected with a vector expressing furin;
b) culturing the cells obtained in a), so as to express the protein. The culturing is carried out according to conventional conditions, well known to those skilled in the art. Preferably, when the cells co-express the protein and furin, the protein produces directly in the activated form; and
c) optionally, purifying the protein obtained.

Here again, such a process directly uses a polynucleotide which does not contain sequence encoding an activation peptide.

The protein according to the invention can be used as a medicament. Consequently, the protein according to the invention can be introduced into a pharmaceutical composition. In particular, the protein according to the invention can be used for the treatment of coagulation disorders, in particular hemorrhagic disorders.

Preferably, the protein according to the invention can be used in the prevention or treatment in a patient, in particular a human being or an animal, of hemorrhagic events induced by taking anticoagulants, which are factor Xa-specific inhibitors. In such a use, the protein according to the invention serves as an antidote to factor Xa-specific inhibitors. The term “antidote” denotes molecules, and in particular proteins, capable of neutralizing or reversing all or part of the anticoagulant activity of anticoagulants. It must be possible for this effect to occur in a more or less short period of time, in relation to the location and the magnitude of the hemorrhagic event in order to slow down, reduce or totally interrupt this hemorrhagic event. The anticoagulant activity of anticoagulants can be measured using overall coagulometric tests (Quick time (PT), activated partial thromboplastin time (aPTT)). In their presence, there is a decrease in the INR (International Normalized Ratio). It is also possible to envision measuring their effects using coagulation tests which measure thrombin generation (TGT).

The term “factor Xa-specific inhibitors” denotes compounds capable of inhibiting, directly or indirectly, the procoagulant activity of FXa which consists of the conversion of prothrombin to thrombin in vitro, and/or ex vivo, and/or in vivo. The FXa inhibitors can be classified as inhibitors of peptide nature, which have been extracted and purified or obtained by genetic engineering, and as inhibitors of non-peptide nature obtained by chemical synthesis (Kher et al., 1998, La Lettre du pharmacologue, 12(6), 222-226). The inhibitors which block the active site of FXa are called direct inhibitors. while the inhibitors which act by binding and by catalyzing the effect of antithrombin with respect to FXa are called indirect inhibitors. Examples of direct peptide inhibitors of FXa include in particular TAP (tick anticoagulant peptide) extracted from tick saliva, antistasin extracted from the salivary glands of the leech Haementeria officinalis, ACAP (ancylostoma caninum anticoagulant peptide) isolated from the hookworm or recombinant forms thereof r Ac AP5, r Ac AP2 (also called NAP-5) or else FXa I (factor Xa inhibitor) extracted from the saliva of the leech Hirudo medicinalis or the recombinant protein corresponding thereto, called Yagin. Among the direct non-peptide inhibitors of FXa are DX 9065a, LY517717 and xabans (eribaxaban, apixaban, betrixaban, edoxaban, otamixaban, rivaroxaban). LMWHs, the oligosaccharides fondaparinux or idraparinux, and heparinoids (danaparoid, sulfodexide, dermatan sulfate) constitute examples of indirect FXa inhibitors.

The use of the protein according to the invention as an antidote to FXa inhibitors allows these inhibitors to be titrated. Preferably, the inhibitory activity of the protein according to the invention on FXa-specific inhibitors is at least approximately 10%, 20%, 30%, 40%, 50%, 60%, 70%, 80%, 90% or 100%.

The pharmaceutical composition of the invention may be combined with pharmaceutically acceptable excipients, and optionally sustained-release matrices, such as biodegradable polymers, so as to form a therapeutic composition.

The pharmaceutical composition of the present invention may be administered orally, sublingually, subcutaneously, intramuscularly, intravenously, intra-arterially, intrathecally, intra-ocularly, intracerebrally, transdermally, via the pulmonary route, locally or rectally. The active ingredient, alone or in combination with another active ingredient, can then be administered in unit administration form, as a mixture with conventional pharmaceutical supports. Unit administration forms include oral forms such as tablets, gel capsules, powders, granules and oral solutions or suspensions, sublingual or buccal administration forms, aerosols, subcutaneous implants, transdermal, topical, intraperitoneal, intramuscular, intravenous, subcutaneous and intrathecal administration forms, intranasal administration forms and rectal administration forms.

Preferably, the pharmaceutical composition contains a carrier that is pharmaceutically acceptable for a formulation capable of being injected. These may in particular be sterile isotonic formulae, saline solutions (with monosodium or disodium phosphate, sodium chloride, potassium chloride, calcium chloride or magnesium chloride and the like, or mixtures of such salts), or lyophilized compositions, which, when sterilized water or physiological saline is added, as appropriate, allow injectable solutes to be formed.

The pharmaceutical forms appropriate for injectable use comprise sterile aqueous solutions or dispersions, oily formulations, including sesame oil and peanut oil, and sterile powders for the extemporaneous preparation of sterile injectable solutions or dispersions. In any event, the form must be sterile and must be fluid since it must be injected using a syringe. It must be stable under the manufacturing and storage conditions and must be preserved against the contaminating action of microorganisms, such as bacteria and fungi.

The dispersions according to the invention can be prepared in glycerol, liquid polyethylene glycols or mixtures thereof, or in oils. Under normal conditions of storage and use, these preparations contain a preservative to prevent the growth of microorganisms.

The pharmaceutically acceptable carrier may be a solvent or dispersing medium containing, for example, water, ethanol, a polyol (for example, glycerol, propylene glycol, polyethylene glycol, and the like), appropriate mixtures thereof, and/or vegetable oils. Suitable fluidity can be maintained, for example, by using a surfactant, such as lecithin. The prevention of the action of microorganisms can be brought about by various antibacterial and antifungal agents, for example parabens, chlorobutanol, phenol, sorbic acid or else thimerosal. In many cases, it will be preferable to include isotonic agents, for example sugars or sodium chloride. Sustained absorption of the injectable compositions can be brought about by using, in the compositions, absorption-delaying agents, for example aluminum monostearate or gelatin.

The sterile injectable solutions are prepared by incorporating the active substances in required amounts into the appropriate solvent with several of the other ingredients listed above, where appropriate followed by filtration sterilization. As a general rule, the dispersions are prepared by incorporating the various sterilized active ingredients into a sterile carrier which contains the basic dispersing medium and the other required ingredients among those listed above. In the case of sterile powders for the preparation of sterile injectable solutions, the preferred preparation processes are vacuum-drying and lyophilization. During the formulation, the solutions will be administered in a manner compatible with the dosage-regimen formulation and in a therapeutically effective amount. The formulations are readily administered in a variety of galenical forms, such as the injectable solutions described above, but drug-release capsules and the like may also be used. For parenteral administration in an aqueous solution for example, the solution must be suitably buffered and the liquid diluent made isotonic with a sufficient amount of saline solution or of glucose. These particular aqueous solutions are particularly suitable for intravenous, intramuscular, subcutaneous and intraperitoneal administration. In this regard, the sterile aqueous media that can be used are known to those skilled in the art. For example, a dose can be dissolved in 1 ml of isotonic NaCl solution and then added to 1000 ml of appropriate liquid, or injected at the proposed site of the infusion. Certain dosage-regimen variations will necessarily have to take place depending on the condition of the subject treated.

The pharmaceutical composition of the invention can be formulated in a therapeutic mixture comprising approximately 0.0001 to 1.0 milligrams, or approximately 0.001 to 0.1 milligrams, or approximately 0.1 to 1.0 milligrams, or even approximately 10 milligrams per dose or more. Multiple doses can also be administered. The level of therapeutically effective dose specific for a particular patient will depend on a variety of factors, including the disorder which is treated and the seriousness of the disease, the activity of the specific compound used, the specific composition used, the patient's age, body weight, general health, sex and diet, the time of the administration, the route of administration, the rate of excretion of the specific compound used, the duration of the treatment, or else the medicaments used in parallel.

The following examples are given for the purpose of illustrating various embodiments of the invention.

The figure legends are the following:

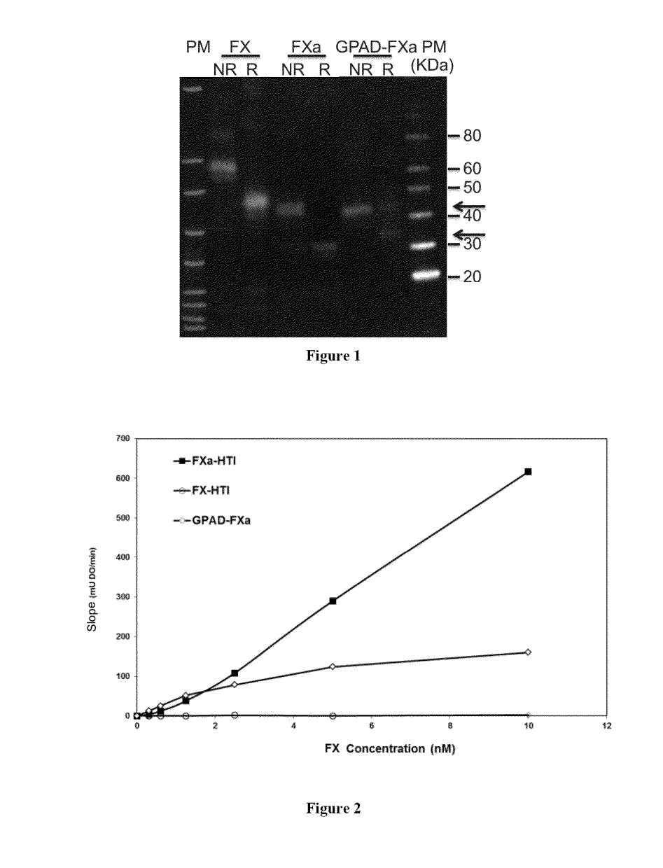

FIG. 1: Analysis of the GPAD-FXA produced in HEK293

The non-activated (FX) or activated (FXa) plasma FX and the GPAD-FXa are revealed by immunoblotting using an anti-FX polyclonal antibody after separation by SDS 4-12% PAGE. The samples were non-reduced (NR) or reduced (R) by means of a β-mercaptoethanol treatment. Two different molecular weight markers (MW) are used. Only the values of the one on the right are indicated. The two arrows indicate the level of migration of the non-reduced GPAD-FXa (upper arrow) and the reduced GPAD-FXa (lower arrow).

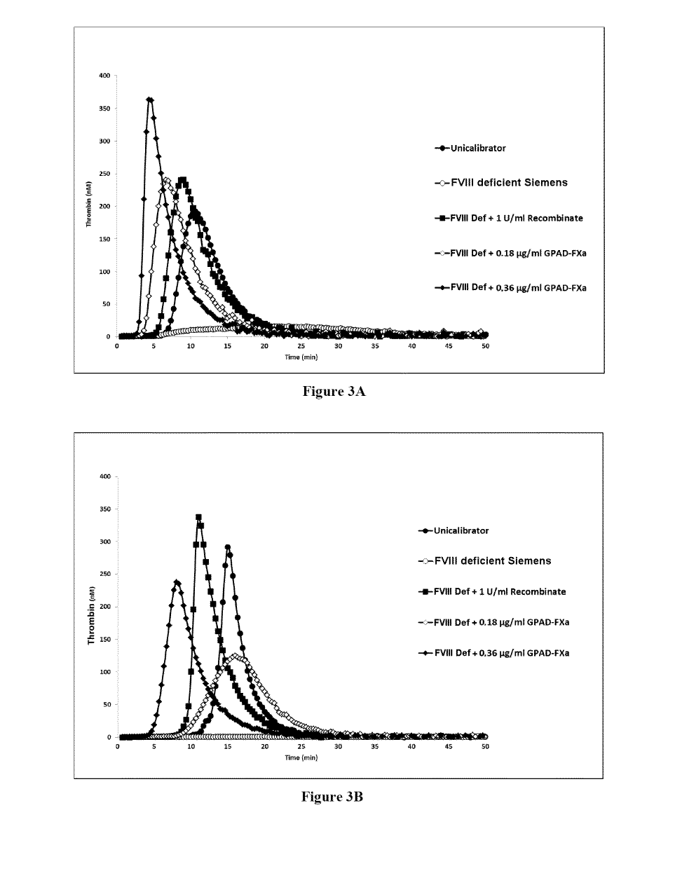

FIG. 2: Amidolytic activity of GPAD-FXa

The amidolytic activity with respect to the substrate Pefachrome 8595, over time, of the GPAD-FXa (white diamonds) is compared with the activity of factor X (white circles) and activated factor X (black squares);
along the x-axis: time (in minutes)
along the y-axis: rate of appearance of the Pefachrome 8595 degradation product in mODU/min.

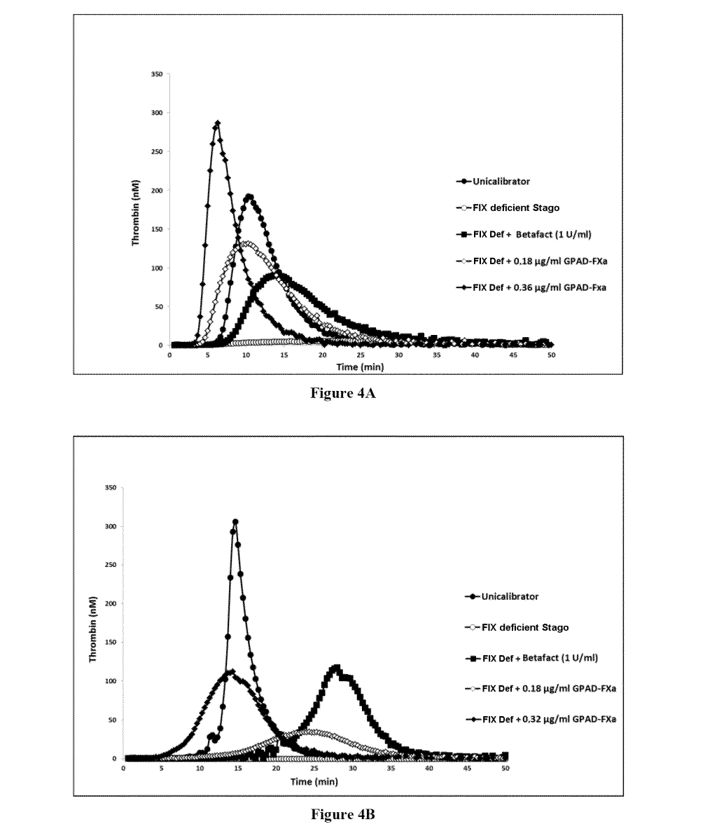

FIG. 3A: Thrombogram obtained from the addition of 0.18 μg/ml or 0.36 μg/ml of GPAD-FXa to a pool of FVIII-deficient plasma following activation with tissue factor:
along the x-axis: time (in minutes)
along the y-axis: maximum thrombin concentration observed (in nM).

The normal plasma pool sample is represented by the curve and the sample corresponding to a factor VIII-deficient plasma by the curve ◯. The curve ▪ represents an FVIII-deficient plasma having received a replacement with 1 U/ml of recombinant FVIII (Recombinate).

The GPAD-FXa was added in a proportion of 0.18 μg/ml (curve ⋄) or 0.36 μg/ml (curve ♦) to the FVIII-deficient plasma sample.

FIG. 3B: Thrombogram obtained from the addition of 0.18 μg/ml or 0.36 μg/ml of GPAD-FXa to a pool of FVIII-deficient plasma following activation with cephalin:
along the x-axis: time (in minutes)
along the y-axis: maximum thrombin concentration observed (in nM).

The normal plasma pool sample is represented by the curve and the sample corresponding to a factor VIII-deficient plasma by the curve ◯. The curve of ▪ represents an FVIII-deficient plasma having received a replacement with 1 U/ml of recombinant FVIII (Recombinate).

The GPAD-FXa was added in a proportion of 0.18 μg/ml (curve ⋄) or 0.36 μg/ml (curve ♦) to the FVIII-deficient plasma sample.

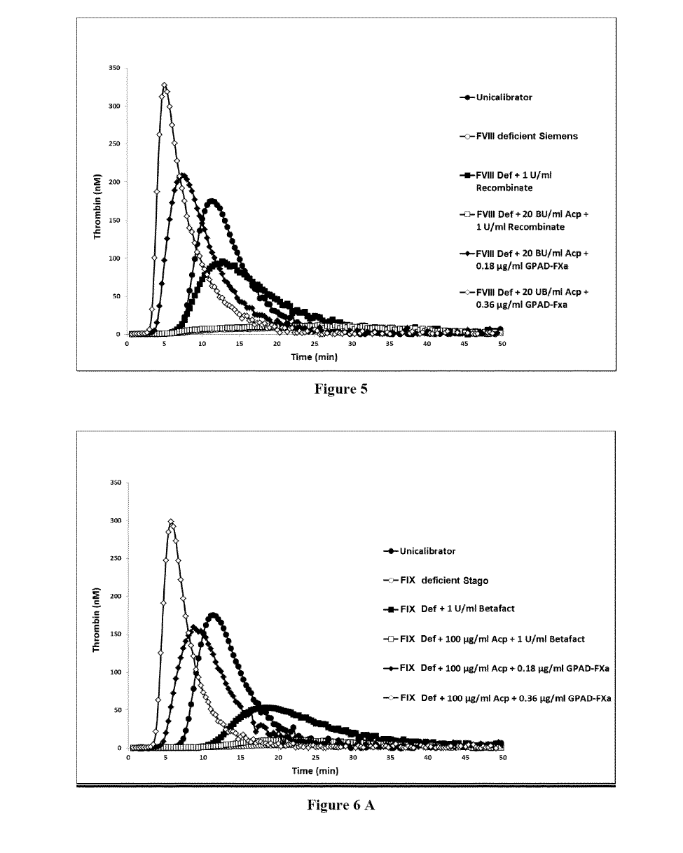

FIG. 4A: Thrombogram obtained from the addition of 0.18 μg/ml or 0.36 μg/ml of GPAD-FXa to a pool of FIX-deficient plasma following activation with tissue factor:
along the x-axis: time (in minutes)
along the y-axis: maximum thrombin concentration observed (in nM).

The normal plasma pool sample is represented by the curve and the sample corresponding to a factor IX-deficient plasma by the curve ◯. The curve ▪ represents an FIX-deficient plasma having received a replacement with 1 U/ml of Betafact (plasma FIX).

The GPAD-FXa was added in a proportion of 0.18 μg/ml (curve ⋄) or 0.36 μg/ml (curve ♦) to the FIX-deficient plasma sample.

FIG. 4B: Thrombogram obtained from the addition of 0.18 μg/ml or 0.36 μg/ml of GPAD-FXa to a pool of FIX-deficient plasma following activation with cephalin:
along the x-axis: time (in minutes)
along the y-axis (maximum thrombin concentration observed (in nM).

The normal plasma pool sample is represented by the curve . The sample corresponding to a factor IX-deficient plasma by the curve ◯. The curve ▪ represents an FIX-deficient plasma having received a replacement with 1 U/ml of Betafact (plasma FIX).

The GPAD-FXa was added in a proportion of 0.18 μg/ml (curve ⋄) or 0.36 μg/ml (curve ♦) to the FIX-deficient plasma sample.

FIG. 5: Thrombogram obtained from the addition of 0.18 μg/ml or 0.36 μg/ml of GPAD-FXa to a pool of FVIII-deficient plasma in the presence of FVIII-inhibiting antibodies following activation with tissue factor:
along the x-axis: time (in minutes)
along the y-axis: maximum thrombin concentration observed (in nM).

The thrombin generation obtained from a normal plasma pool sample is represented by the curve , and from a factor VIII-deficient plasma by the curve ◯. The curve ▪ represents an FVIII-deficient plasma having received a replacement with 1 U/ml of recombinant FVIII (Recombinate), and the curve □ represents the same sample in the presence of anti-FVIII polyclonal antibodies having an inhibitory activity of 20 Bethesda units/ml. The curves ♦ and ⋄ represent the signal of an FVIII-deficient plasma containing anti-FVIII polyclonal antibodies having an inhibitory activity of 20 Bethesda units/ml in the presence, respectively, of 0.18 and 0.38 μg/ml of GPAD-FXa.

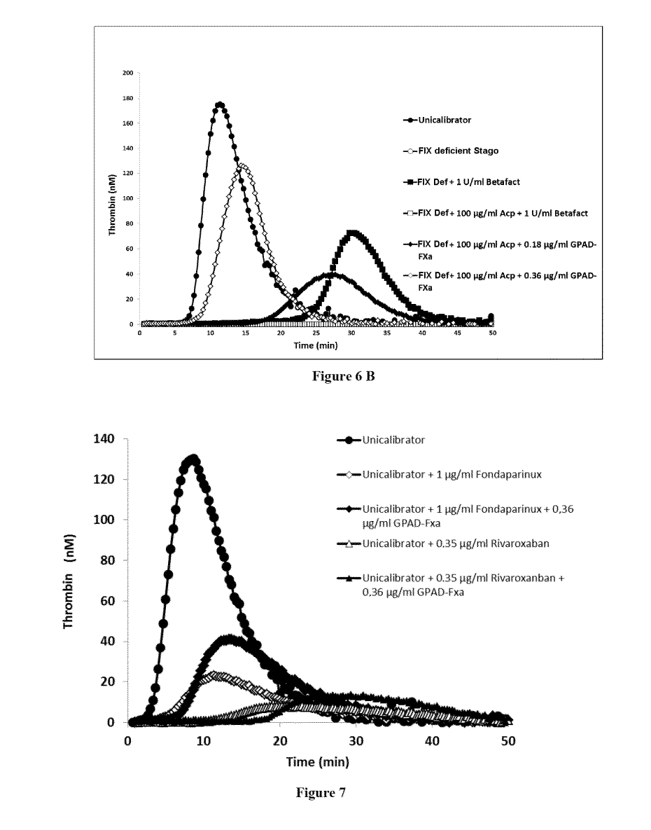

FIG. 6A: Thrombogram obtained from the addition of 0.18 μg/ml or 0.36 μg/ml of GPAD-FXa to a pool of FIX-deficient plasma in the presence of FIX-inhibiting antibodies following activation with tissue factor:
along the x-axis: time (in minutes)
along the y-axis: maximum thrombin concentration observed (in nM).

The thrombin generation obtained from a normal plasma pool sample is represented by the curve , and from a factor IX-deficient plasma by the curve ◯. The curve ▪ represents an FIX-deficient plasma having received a replacement with 1 U/ml of plasma FIX (Betafact), and the curve □ represents the same sample in the presence of anti-FIX polyclonal antibodies (100 μg/ml). The curves ♦ and ⋄ represent the signal of an FIX-deficient plasma containing anti-FIX polyclonal antibodies in the presence, respectively, of 0.18 and 0.38 μg/ml of GPAD-FXa.

FIG. 6B: Thrombogram obtained from the addition of 0.18 μg/ml or 0.36 μg/ml of GPAD-FXa to a pool of FIX-deficient plasma in the presence of FIX-inhibiting antibodies following activation with cephalin:
along the x-axis: time (in minutes)
along the y-axis: maximum thrombin concentration observed (in nM).

The thrombin generation obtained from a normal plasma pool sample is represented by the curve , and from a factor IX-deficient plasma by the curve ◯. The curve ▪ represents an FIX-deficient plasma substituted with 1 U/ml of plasma FIX (Betafact), and the curve □ represents the same sample in the presence of anti-FIX polyclonal antibodies (100 μg/ml). The curves ♦ and ♦ represent the signal of an FIX-deficient plasma containing anti-FIX polyclonal antibodies in the presence, respectively, of 0.18 and 0.38 μg/ml of GPAD-FXa.

FIG. 7: Thrombograms obtained from the addition of 0.36 μg/ml of GPAD-FXa in FVIII-deficient plasma containing Fondaparinux (1 μg/ml) or Rivaroxaban (0.35 μg/ml) following activation with cephalin:
along the x-axis: time (in minutes)
along the y axis: maximum thrombin concentration observed (in nM).

The normal plasma pool sample is represented by the curve . The curves (♦) and (⋄) represent respectively the effect of the addition of Fondaparinux (1 μg/ml) to the plasma with or without GPAD-FXa (0.36 μg/ml). The curves (▴) and (Δ) represent respectively the effect of the addition of Rivaroxaban (0.35 μg/ml) to the plasma with or without GPAD-FXa (0.36 μg/ml).

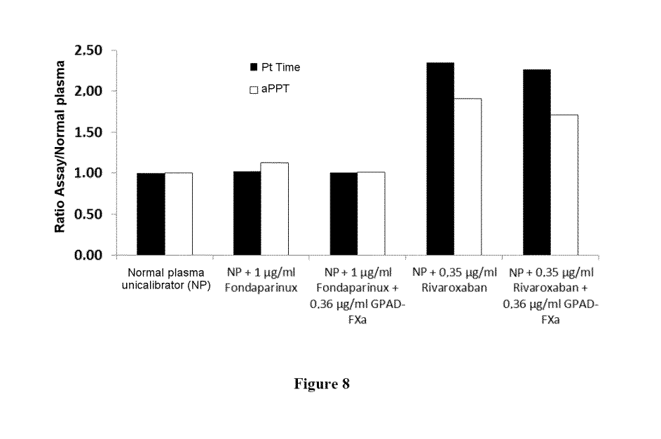

FIG. 8: Effect of the presence of GPAD-FXa on the prothrombin time and activated partial thromboplastin time in the presence of coagulation inhibitors

The prothrombin time (Pt; black column) and activated partial thromboplastin time (aPPT; white column) were measured on normal plasma or plasma inhibited with Fondaparinux (1 μg/ml) or Rivaroxaban (0.35 μg/ml) in the presence or absence of GPAD-FXa (0.36 μg/ml).

FIG. 9: GPAD1 and GPAD2 constructs, and optimized versions thereof

The GPAD 1 and GPAD 2 constructs and optimized version thereof are represented diagrammatically, as are two GPAD 3 constructions containing a linker of variable size (L1 or L2). The GPAD1-FXa and GPAD2-FXa sequences differ by the addition of an additional furin site in GPAD2-FXa downstream of the FX activation site (RKR in bold, double arrow). The light-chain and heavy-chain separation site is indicated by the yellow arrow. In GPAD3, L1 is a linker of variable size and composition which ends with an arginine at the C-terminal end; L2 is a linker of variable size and composition which begins with the sequence RKR and ends at the C-terminal end with an arginine.

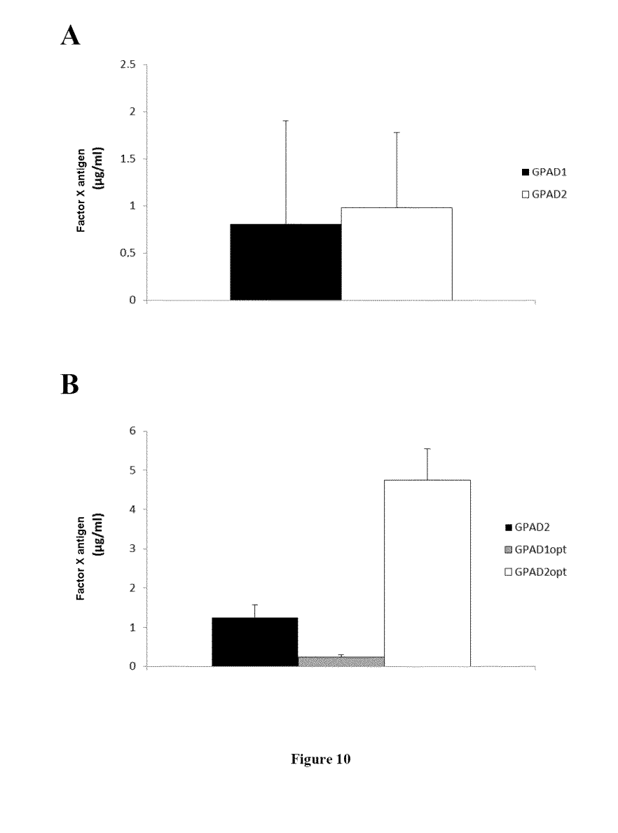

FIG. 10: Comparison of the productions of the various GPAD-FXa molecules in HEK293

Production (in μg/ml) of the GPAD1-FXa and GPAD2-FXa molecules (panel A) and GPAD2-FXa, GPAD1opt-FXa and GPAD2opt-FXa molecules (panel B) in the HEK293 line. The productions are a mean of values obtained following various transfections using various expression vectors (pCEP4, OptiHEK, pTT) and following various production times (7 or 11 days). Panel A: GPAD1-FXa, n=6; GPAD2-FXa, n=20; Panel B: GPAD2-FXa, n=4; GPAD1opt-FXa, n=7; GPAD2opt-FXa, n=4.

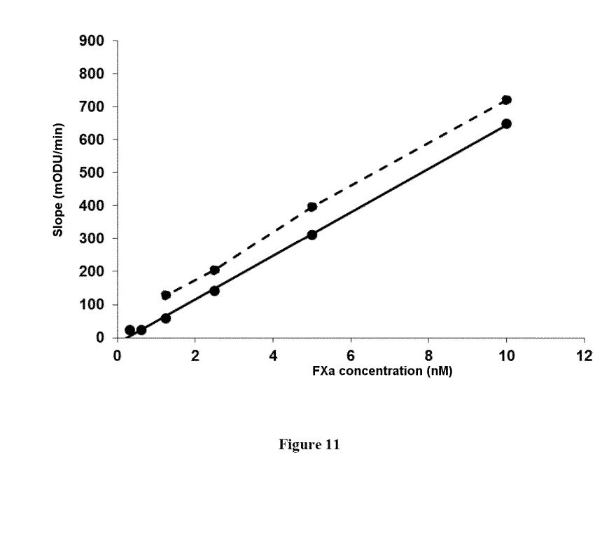

FIG. 11: Chromogenic activity of GPAD2opt-FXa

The GPAD2opt-FXa was purified according to a protocol using a heparin column followed by a phenyl-sepharose column. The amidolytic activity with respect to the substrate Pefachrome 8595, over time, of GPAD2opt-FXa (dashed curve) is compared with the activity of activated factor X (solid curve).
along the x-axis: time (in minutes)
along the y axis: rate of appearance of the Pefachrome 8595 degradation product in mODU/min.

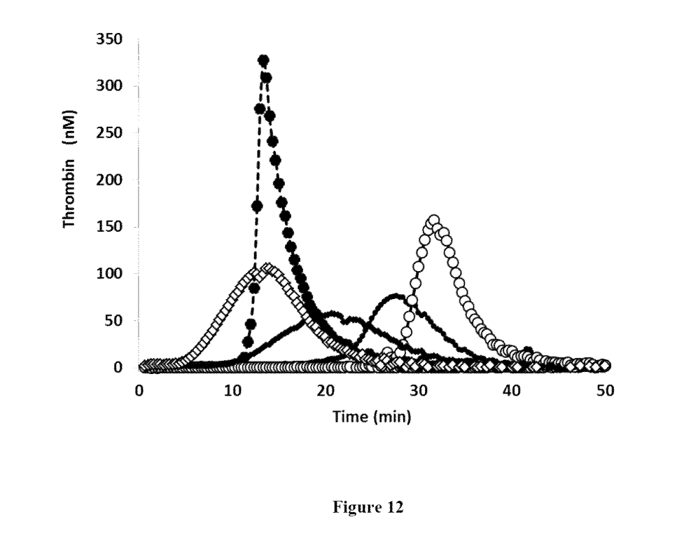

FIG. 12: Thrombograms obtained from the addition of GPAD2opt-FXa optionally produced in the presence of furin, to a pool of FVIII-deficient plasma following activation with cephalin:
along the x-axis: time (in minutes)
along the y axis: maximum thrombin concentration observed (in nM).

The normal plasma pool sample is represented by the curve and the sample corresponding to a factor VIII-deficient plasma by the non-connected curve ◯. The dashed curve represents an FVIII-deficient plasma having received a replacement with 1 U/ml of recombinant FVIII (Recombinate) and the connected curve of ◯ represents an FVIII-deficient plasma having received a replacement with 0.1 U/ml. The GPAD2op-FXa (2 μg/ml) produced with furin (curve ⋄) or without furin (curve ♦) were analyzed in FVIII-deficient plasma.

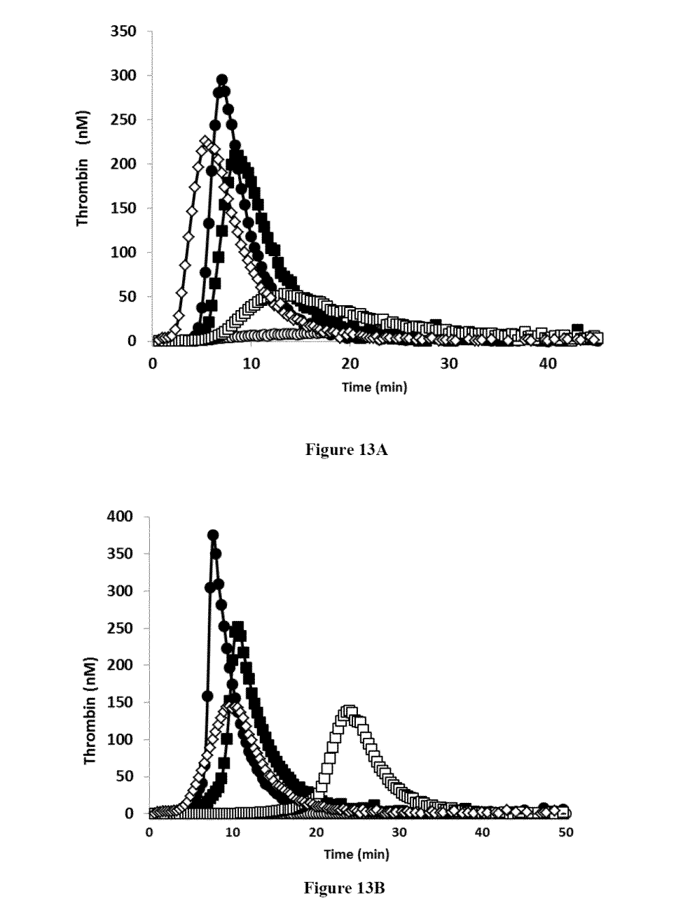

FIG. 13: Thrombograms obtained from the addition of GPAD2opt-FXa to a pool of FVIII-deficient plasma:
along the x-axis: time (in minutes)
along the y axis: maximum thrombin concentration observed (in nM).

The normal plasma pool sample is represented by the curve and the sample corresponding to a factor FVIII-deficient plasma by the curve ◯. In panel A, the coagulation is stimulated by the addition of a mixture of 0.5 nM TF/4 μM PL. In panel B, the stimulation is initiated with cephalin. The curve ▪ represents an FVIII-deficient plasma having received a replacement with 1 U/ml of Recombinate (Baxter recombinant FVIII) and the curve □ represents an FVIII-deficient plasma having received a replacement with 0.1 U/ml. The GPAD2op-FXa (2 μg/ml, curve ⋄) produced in the presence of furin was analyzed in FVIII-deficient plasma.

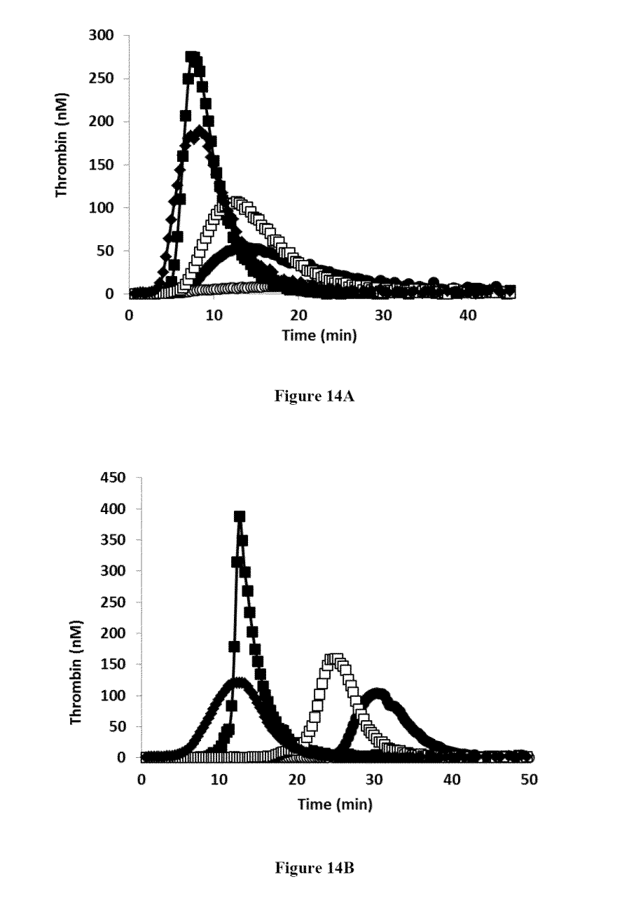

FIG. 14: Thrombograms obtained from the addition of GPAD2opt-FXa to a pool of FIX-deficient plasma:
along the x-axis: time (in minutes)
along the y axis: maximum thrombin concentration observed (in nM).

The normal plasma pool sample is represented by the curve and the sample corresponding to a factor IX-deficient plasma by the curve ◯. In panel A, the coagulation is stimulated by the addition of a mixture of 0.5 nM TF/4 μM PL. In panel B, the stimulation is initiated with cephalin. The curve ▪ represents an FIX-deficient plasma having received a replacement with 1 U/ml of FIX (Betafact) and the curve □ represents an FIX-deficient plasma having received a replacement with 0.1 U/ml. The GPAD2op-FXa (2 μg/ml, curve ♦) produced in the presence of furin was analyzed in FIX-deficient plasma.

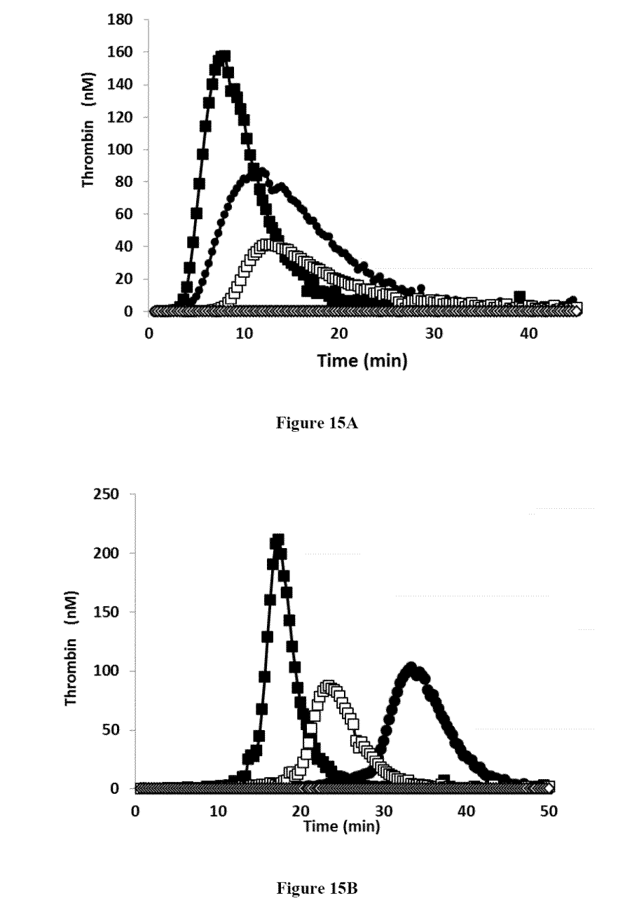

FIG. 15: Thrombograms obtained from the addition of GPAD2opt-FXa to a pool of FX-deficient plasma:
along the x-axis: time (in minutes)
along the y axis: maximum thrombin concentration observed (in nM).

The normal plasma pool sample is represented by the curve and the sample corresponding to a factor X-deficient plasma is represented by the curve ◯. The latter curve is not distinguishable since it is located at the level of the x-axis. In panel A, the coagulation is stimulated by the addition of a mixture of 0.5 nM TF/4 μM PL. In panel B, the stimulation is initiated with cephalin. The curve ▪ represents an FX-deficient plasma having received a replacement with 10 μg/ml of plasma FX (Cryopep) and that of the □ represents an FX-deficient plasma having received a replacement with 1 μg/ml. The GPAD2op-FXa (2 μg/ml, curve ⋄) produced in the presence of furin was analyzed in FX-deficient plasma.

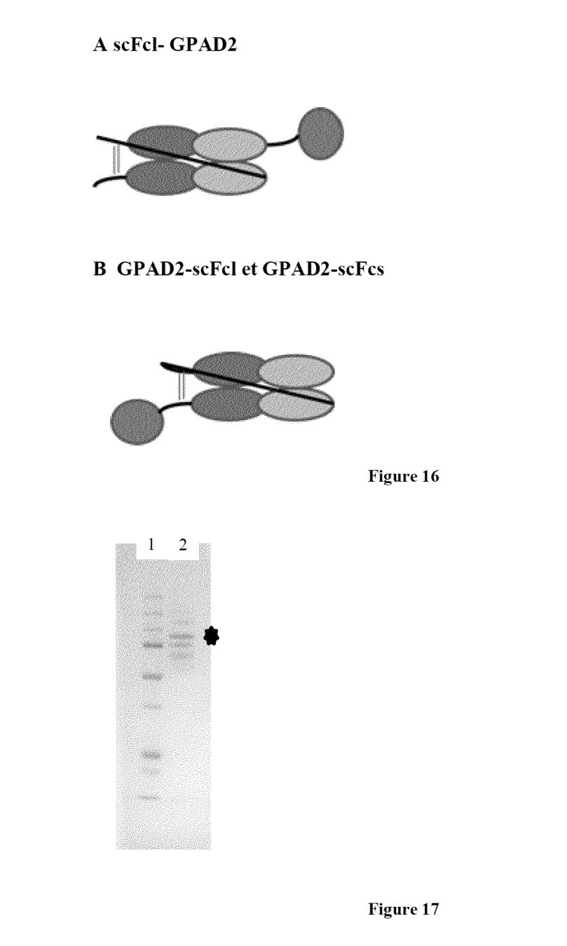

FIG. 16: Diagram of the constructs of the GPAD2opt-FXa molecules fused to the scFc molecules

Diagrammatic representation of the GPAD2opt-scFcs and GPAD2opt-scFcl molecules (A) and scFcl-GPAD2opt-FXa molecules (B). The orange and red ellipses represent the CH3 and CH2 domains respectively of gamma immunoglobulins. The linkers are represented by the black lines. The GPAD2opt-FXa domain is represented by a blue circle. In the GPAD2opt-scFcs and GPAD2opt-scFcl constructs, the GPAD2opt-FXa domain is cloned in the N-terminal position relative to the scFc fragment. In the scFcl-GPAD2opt-FXa construct, it is cloned in the C-terminal position. The difference between GPAD2opt-scFcs and GPAD2opt-scFcl lies in the size of the linker which is not shown in this figure.

FIG. 17: Purification of scFcl-GPAD2opt-FXa

The scFcl-GPAD2opt-FXa molecule (900 μg) produced by transient expression in HEK was purified on protein A gel and then analyzed after SDS-PAGE separation and staining with Nupage Blue. 1, molecular markers; 2, purified scFcl-GPAD2opt; *, expected molecular weight for scFcl-GPAD2opt-FXa.

FIG. 18: Thrombograms obtained from the addition of GPAD2opt-FXa or scFcl-GPAD2opt-FXa to a pool of FVIII-deficient plasma following activation with cephalin:
along the x-axis: time (in minutes)
along the y axis: maximum thrombin concentration observed (in nM).

The samples of a pool of plasma deficient in factor VIII and having received a replacement with 1 U/ml or 0.1 U/ml of FVIII are represented by the curves and ◯ respectively. The effect of GPAD2op-FXa (2 μg/ml, curve ♦) and of scFcl-GPAD2op-FXa (4 μg/ml, curve ▴) was analyzed in FVIII-deficient plasma after activation with cephalin.

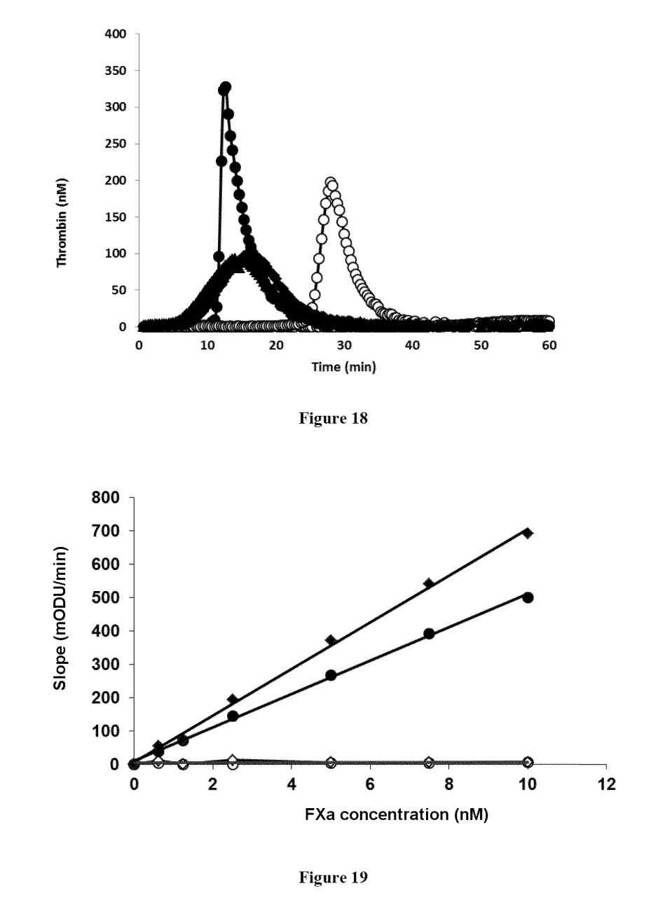

FIG. 19: Inhibition of the chromogenic activity of scFcl-GPAD2opt-FXa by Rivaroxaban

The chromogenic activities, on the substrate Pefachrome 8595, of FXa of plasma origin (curve ) or of scFcl-GPAD2opt-FXa (curve ♦) were measured as described in example 1. These activities were also measured in the presence of Rivaroxaban (0.35 μg/ml). The results of the FXa of plasma origin are represented by the curve ◯ and for scFcl-GPAD2opt-FXa by the curve ⋄.

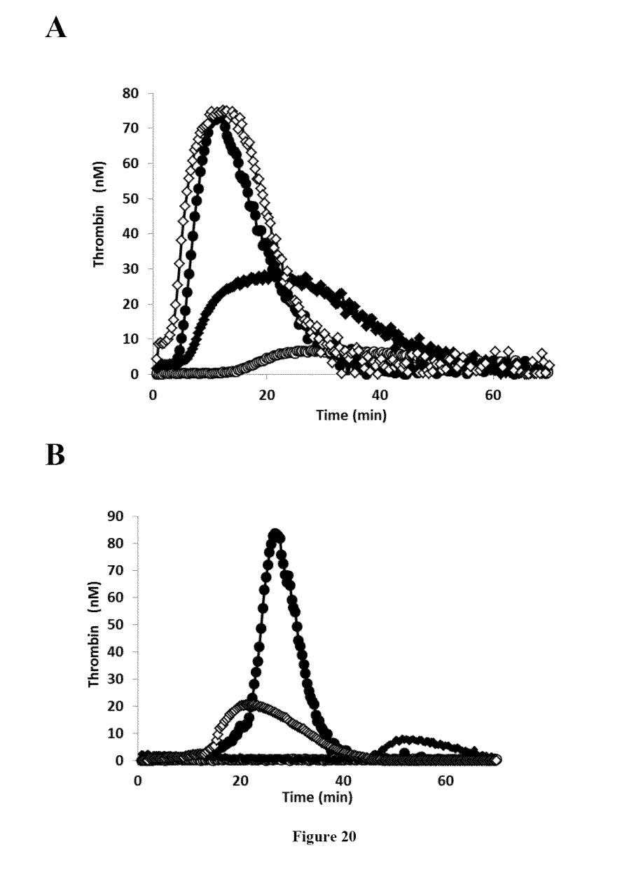

FIG. 20: Thrombograms obtained from the addition of scFcl-GPAD2opt-FXa to a normal plasma supplemented with Rivaroxaban
along the x-axis: time (in minutes)
along the y axis: maximum thrombin concentration observed (in nM).

The normal or Rivaroxaban-supplemented (0.25 μg/ml) plasma pool samples are represented by the curves and ◯ respectively. The effect of the addition of increasing doses of scFcl-GPAD2opt-FXa (25 or 50 μg/ml) to normal plasma supplemented with Rivaroxaban (0.25 μg/ml) is visualized respectively by the curves: ♦ and ⋄. Panel A, initiation of the TGT with the 0.5 pM TF/4 μM PL mixture. Panel B, initiation of the TGT with cephalin.

### EXAMPLES

### Example 1

### Preparation of GPAD-FXa

### Materials:

The activated human plasma factor X or non-activated human plasma factor X (FXa/FX), the chromogenic substrates pNAPEP 1025 and Pefachrome 8595, and the hemophilia A plasma with inhibitors and the anti-FX and anti-FIX polyclonal antibodies come from Haematologic Technologies (Cryopep Montpellier, France).

The control recombinant factor VIII (Recombinate) comes from Baxter. The plasma factor IX comes from LFB. The FVIII-deficient plasmas come from Siemens, the FIX-deficient plasmas from Stago.

The anti-FVIII polyclonal antibodies come from Cedarlane.

### Methods:

Production of the GPAD-FXa cDNA

The cDNA encoding a factor X missing the gamma-carboxyglutamic domain and tagged with the HPC4 sequence (see patent FR 11 51637 filed on Mar. 1, 2011) will be used as a template for generating GPAD-FXa. The deletion of the activation peptide and the replacement thereof with a furin site (RKR) is provided by the use of the forward primer (5′-ctggaacgcaggaagaggaggaagaggatcgtgggaggc, SEQ ID No.: 17) and reverse primer (5′-: gcctcccacgatcctcttcctcctcttcctgcgttccag, SEQ ID No.: 18). The cDNA is modified with the Quickchange method (Stratagene) and the pFU Ultra enzyme.

### Recombinant GPAD-FXa Production

On the day before the transfection, HEK 293F cells are subcultured at a cell concentration of 7×10⁵ vc/ml. The cell density and the cell viability are determined on the day of the transfection. The culture volume corresponding to 30×10⁶ cells is centrifuged. The supernatant is removed and the cell pellet is taken up in 28 ml of F17 culture medium (Invitrogen), transferred into a 250 ml Erlenmeyer flask and incubated at 37° C. A transfection agent/DNA complex in a 2:1 ratio is formed. The transfection agent and the DNA corresponding to the vector containing one of the sequences of the GPAD-FXa are prepared in OptiMEM medium (Invitrogen) in the following way:
Addition of 30 μg of DNA to 1 ml of OptiMEM
Addition of 60 μl of transfection agent to 1 ml of OptiMEM.

These two preparations are incubated separately for 5 minutes at ambient temperature and then the solution containing the transfection agent is added to the one containing the DNA. The mixture is incubated for 25 minutes at ambient temperature before being added to the 28 ml of HEK 293F cells. The cells are then incubated at 37° C. with shaking at 125 rpm.

A vector expressing GFP (Green Fluorescent Protein; positive control for transfection) is also transfected under the same conditions. The transfection efficiency is determined 24 h post-transfection by fluorescence microscopy by establishing the ratio of the number of cells expressing GFP to the total number of cells. The cells are maintained in culture for 7 days. The cell density and the viability are determined every day between the 5th and 7th days using an automated cell culture analysis device (Cedex, Innovatis—Roche Applied Science). The viability measurement is based on counting the cells that have incorporated trypan blue. On the 7th day, the cells are centrifuged at 3000 g for 15 minutes. The cell pellet is removed and the cell supernatant containing the recombinant GPAD-FXa is filtered through 0.22 μm and then frozen at −20° C. Before use, the supernatants are concentrated up to 10-fold on Vivaspin 10 kDa columns.

### Visualization of Proteins Following SDS-PAGE Separation

For each gel, at least one appropriate molecular weight control is used. An identical amount of protein to be detected is then loaded into each well in 2× concentrated loading buffer (1M Tris HCl, pH 6.8; 20% SDS; 20% glycerol; 0.1% bromophenol blue). The reduced samples are treated with loading buffer in the presence of 0.5 M β-mercaptoethanol. The NuPAGE MOPS SDS 1× running buffer (Invitrogen) is used for the migration. The samples are left to migrate at 200 V for 50 minutes in a NuPAGE Novex Bis-Tris 4-12% gel.

When the electrophoresis has finished, the proteins separated on the gel are immunodetected after electrotransfer onto a nitrocellulose or PVDF membrane in Towbin buffer (25 mM Tris, 192 mM glycine, 3.5 mM SDS, 10% ethanol) by applying a current of 0.8 mA/cm² for 1 h30. The membrane is then rinsed in water and incubated in saturation buffer (PBS, 1% bovine serum albumin, 0.05% Tween-20). The membrane is then incubated in saturated buffer in the presence of the sheep anti-human FX antibody (Cryopep 9-PAHFX-S) at 2 μg/ml for 1 h and then in the presence of the secondary antibody (anti-sheep IgG H+L peroxidase—Jackson Laboratory) at 1/10000th for 1 h following 4 washes with a solution of physiological saline (0.9% NaCl) containing 0.05% of Tween-20. A protocol of identical washes is applied before visualization. The immunoblot is visualized by chemiluminescence using the SuperSignal West Pico chemiluminescent substrate kit (Pierce).

Measurement of the Factor X Concentration The measurement of the concentration of the FX, FXa and GPAD-FXa is carried out using the Zymutest Factor X commercial kit (Hyphen Biomed). The ELISA gives a linear signal for concentrations ranging from 200 ng/ml to 1.5 ng/ml of plasma factor X. The three proteins are recognized in an identical manner by the kit.

### Measurement of the Chromogenic Activity of the Activated Factor X

The measurement of the activity of FX, FXa and GPAD-FXa was studied at 37° C. in stop buffer (50 mM Tris, pH 8.8, 0.475 M NaCl, 9 mM EDTA). The chromogenic activity of the FXa at various concentrations was monitored over time by measuring the hydrolysis of the substrate Pefachrome 8595 (250 μM) at 405 nm.

### Protocol for Measuring Thrombin Generation:

The Fluoroskan Ascent, all the reagents after reconstitution and the samples are preheated to 37° C. The 0.5 pM TF/4 μM PL (final concentration) and cephalin (16.7% final concentration) reagents will be used as inducers of the reaction. The “Thrombin Calibrator” reagent (Diagnostica Stago) is systematically used as assay control. Each test sample (80 μl) is studied in duplicate in the presence of 20 μl of the TF/PL mixture or 20 μl of cephalin. The fluorogenic substrate (20 μl; FluCa kit (Diagnostica Stago) is then added and the appearance of thrombin is monitored by excitation at 390 nm and emission at 460 nm for 60 min. The results are then analyzed using the Thrombinoscope software (Stago, Asnières, France).

### Example 2

### Analysis of the GPAD-FXa Produced in HEK293

The culture supernatants of the cells transfected with an empty vector or a vector encoding GPAD-FXa were assayed with the Zymutest Factor X kit (Hyphen Biomed). No significant amount of FX was detected in the control media. On the other hand, in the media of the cells expressing GPAD-FXa, levels of 0.11 to 0.44 μg/ml were assayed. These concentrations vary according to the various assays and the production volumes. These data indicate that GPAD-FXa is produced by the HEK293 cells and that it is recognized by the factor X detection kit.

In order to confirm the presence and to visualize the quality of the GPAD-FXa produced in the HEK293 supernatant, an aliquot of concentrated medium was analyzed by SDS-PAGE on an acrylamide gradient (FIG. 1). Identical amounts of proteins were loaded onto the gel, and the signals obtained by immunoblotting are very close, suggesting that the GPAD-FXa quantification by ELISA is correct. The samples were loaded having been reduced (R) with β-mercaptoethanol or having not been reduced (NR). They were compared with the plasma FX and plasma FXa treated in an identical manner. The non-reduced GPAD-FXa migrates in a single molecular form at an apparent molecular weight of 42.1 kDa corresponding to the expected weight (upper arrow). After reduction, the GPAD-FXa migrates in two molecular forms, one at the same molecular weight as the non-reduced form and the other at a molecular weight of 35.6 kDa (lower arrow). The latter form migrates in the same way as the heavy chain of FXa. These results suggest that the GPAD-FXa is produced in two molecular forms: a non-cleaved single-chain form of 42.1 kDa and a form in which the heavy and light chains are cleaved and held by disulfide bridge.

### Example 3

### Amidolytic Activity of GPAD-FXa

The immunoblotting results suggest that the molecule is produced at least partially in its active form. In order to confirm this hypothesis and to verify that the catalytic site of the GPAD-FXa has remained functional, the amidolytic activity of the molecule is measured. Various concentrations of GPAD-FXa are incubated for 5 min at 37° C. in the presence of the chromogenic substrate Pefachrome 8595, specific for FXa. The initial rate of appearance of the substrate (in mODU/min) is determined according to the initial concentration of FX(a) or GPAD-FXa (FIG. 2). As expected, the non-activated FX (curve ◯) does not cleave the substrate, unlike the FXa (curve ▪). From 0 to 10 nM of FXa, the rate of appearance of the substrate increases in a linear manner. GPAD-FXa (curve ⋄) also shows a capacity to cleave Pefachrome 8595. This capacity is, however, lower than that of FXa. However, the presence in the supernatant of GPAD-FXa molecules not activated by furin, and therefore devoid of enzymatic activity, may explain this quantitative difference.

### Example 4

### GPAD-FXa Corrects an FVIII Deficiency

The capacity of the GPAD-FXa to restore thrombin generation in FVIII-deficient plasma was evaluated following induction with tissue factor (FIG. 3A) or cephalin (FIG. 3B). Under the experimental conditions, the FVIII-deficient plasma (Siemens) does not make it possible to generate thrombin (curve ◯). The supplementation of this plasma with 1 unit/ml of FVIII (Recombinate, Baxter) enables a significant generation of thrombin (curve ▪). The total amount of thrombin generated is 1579 nM of thrombin (table I). The addition of culture supernatant containing the GPAD-FXa at 0.18 μg/ml (1.8% of the plasma concentration of FX, i.e. 0.018 U/ml) makes it possible to restore a thrombin production (curve ⋄). The amount of thrombin generated by GPAD-FXa (ETP) is about 1545 nM corresponding to 98% of the thrombin generation by normalizing replacement FVIII. Moreover, the presence of GPAD-FXa also makes it possible to normalize the thrombin appearance time with a time to reach the summit of the peak at 6.83 min compared with 8.67 min for the plasma having received replacement. Twice the dose of GPAD-FXa (0.36 μg/ml) even has a greater effect than the normalizing FVIII with an amount of generated thrombin of 1651 nM and a peak appearance time shortened to 4.83 min. The Unicalibrator positive control (Stago) makes it possible to verify the validity of the experiment by the generation of thrombin in a healthy plasma (curve ). A similar experiment is carried out by inducing the thrombin generation with cephalin. Under these conditions, the FVIII-deficient plasma does not make it possible to generate thrombin (curve ◯). Conversely, deficient plasma having received FVIII replacement generates 1472 nM of thrombin (curve ▪). The presence of GPAD-FXa at 0.18 μg/ml makes it possible to generate an amount of thrombin corresponding to 1160 nM (curve ⋄; 79% of FVIII). The signal depends on the dose added since a double dose of GPAD-FXa (0.36 μg/ml) makes it possible to obtain 1316 nM of thrombin (curve ♦; 89% of FVIII). The Unicalibrator positive control (Stago) makes it possible to verify the validity of the experiment via the generation of thrombin in a healthy plasma (curve ).

TABLE I
Kinetic parameters of thrombin generation in FVIII-deficient plasma
activated with tissue factor

| | | FVIII | FVIII Def + | FVIII Def + | FVIII Def + |
|---|---|---|---|---|---|
| | | deficient | Recombinate | GPAD-FXa | GPAD-FXa |
| Group name | Unicalibrator | Siemens | (1 U/ml) | (0.16 μg/ml) | (0.32 μg/ml |
| Lagtime (min) | 7.5 | 5.67 | 5.83 | 4 | 3 |
| ETP (nM) | 1293 | 400 | 1579 | 1545.5 | 1651 |
| Peak (nM) | 191.57 | 15.34 | 240.83 | 239.96 | 352.7 |
| ttPeak (min) | 10.83 | 22.5 | 8.67 | 6.83 | 4.83 |

TABLE II
Kinetic parameters of thrombin generation in FVIII-deficient plasma
activated with cephalin

| | | FVIII | FVIII Def + | FVIII Def + | FVIII Def + |
|---|---|---|---|---|---|
| | | deficient | Recombinate | GPAD-FXa | GPAD-FXa |
| Group name | Unicalibrator | Siemens | (1 U/ml) | (0.16 μg/ml) | (0.32 μg/ml |
| Lagtime (min) | 12.83 | 1.17 | 9.67 | 10.17 | 5.5 |
| ETP (nM) | 1139 | 0 | 1472.5 | 1160.5 | 1316 |
| Peak (nM) | 279.72 | 0.76 | 321.76 | 123.12 | 236.63 |
| ttPeak (min) | 15.5 | 32 | 11.33 | 16.5 | 8.33 |

### Example 5

### GPAD-FXa Corrects an FIX Deficiency

The capacity of the GPAD-FXa to restore thrombin generation in FIX-deficient plasma was evaluated following induction with tissue factor (FIG. 4A) or cephalin (FIG. 4B). Under the experimental conditions, the FIX-deficient plasma (Stago) does not make it possible to generate thrombin (curve ◯). The supplementation of this plasma with 1 unit/ml of FIX (Betafact, LFB) enables significant thrombin generation (curve ▪). The total amount of thrombin generated is 1123 nM of thrombin (table III). The addition of culture supernatant containing the GPAD-FXa at 0.18 μg/ml (1.8% of the plasma concentration of FX, i.e. 0.018 U/ml) makes it possible to restore a thrombin production (curve ⋄). The amount of thrombin generated by GPAD-FXa (ETP) is about 1395 nM corresponding to 124% of the thrombin generation by normalizing replacement FIX. Moreover, the presence of GPAD-FXa also makes it possible to normalize the thrombin appearance time with a time to achieve the summit of the peak at 9.67 min compared with 14.17 min for the plasma having received replacement. Twice the dose of GPAD-FXa (curve ♦; 0.36 μg/ml) has an even greater effect than the normalizing FIX with an amount of thrombin generated of 1437 nM and a peak appearance time shortened to 6.17 min. The Unicalibrator positive control (Stago) makes it possible to verify the validity of the experiment via the thrombin generation in a healthy plasma (curve ). A similar experiment is carried out by inducing thrombin generation with cephalin. Under these conditions, the FIX-deficient plasma does not make it possible to generate thrombin (curve ◯). Conversely, deficient plasma having received FIX replacement generates 1030 nM of thrombin (curve ▪). The presence of GPAD-FXa at 0.18 μg/ml makes it possible to generate an amount of thrombin corresponding to 493 nM (curve ⋄; 48% of FIX). The signal depends on the dose added since a double dose of GPAD-FXa (0.36 μg/ml) makes it possible to obtain 1032 nM of thrombin (curve ♦; 100% of FIX). This dose of GPAD-FXa also makes it possible to shorten the time to peak from 28 to 14 min. The Unicalibrator positive control (Stago) makes it possible to verify the validity of the experiment via thrombin generation in a healthy plasma (curve ).

TABLE III
Kinetic parameters of thrombin generation in FIX-deficient plasma
activated with tissue factor

| | | FIX- | FIX Def + | FIX Def + | FIX Def + |
|---|---|---|---|---|---|
| Group | Uni- | deficient | Betafact | GPAD-FXa | GPAD-FXa |
| name | calibrator | Stago | (1 U/ml) | (0.18 μg/ml) | (0.32 μg/ml) |
| Lagtime | 7 | 5.17 | 8.33 | 5 | 3.83 |
| ETP | 1343.5 | 172 | 1123.5 | 1395.5 | 1437 |
| Peak | 190.37 | 5.85 | 90.61 | 132.02 | 279.63 |
| ttPeak | 10.67 | 23.83 | 14.17 | 9.67 | 6.17 |

TABLE IV
Kinetic parameters of thrombin generation in FIX-deficient plasma
activated with cephalin

| | | FIX- | FIX Def + | FIX Def + | FIX Def + |
|---|---|---|---|---|---|
| Group | Unicali- | deficient | Betafact | GPAD-FXa | GPAD-FXa |
| name | brator | Stago | (1 U/ml) | (0.18 μg/ml) | (0.36 μg/ml) |
| Lagtime | 12.83 | 0 | 20 | 14.17 | 7.67 |
| (min) | | | | | |
| ETP (nM) | 1161 | 0 | 1030 | 493.5 | 1032.5 |
| Peak (nM) | 293.25 | 0 | 112.68 | 34.47 | 109.16 |
| ttPeak | 15 | 0 | 28.17 | 24.33 | 14 |
| (min) | | | | | |

### Example 6

### GPAD-FXa Corrects an FVIII Deficiency in the Presence of FVIII-Inhibiting Antibodies

An experiment similar to example 4 was carried out, but with factor VIII-inhibiting antibodies being added to the plasma (FIG. 5). The antibodies were titered and a dose equivalent to 20 Bethesda units/ml was added. This dose is capable of inhibiting any thrombin generation induced by 1 U/ml of FVIII (curve □). In the presence of these antibodies, GPAD-FXa at 0.18 μg/ml (curve ♦) or 0.36 μg/ml (curve ⋄) is capable of restoring a thrombin generation greater than the FVIII-deficient plasma having received FVIII replacement (1 U/ml; curve ▪) but also than a normal plasma ().

It should be noted that the peak times are decreased in the presence of GPAD-FXa compared with the various controls.

### Example 7

### GPAD-FXa Corrects an FIX Deficiency in the Presence of FIX-Inhibiting Antibodies

An experiment similar to example 6 was carried out, but with factor IX-inhibiting antibodies being added to FIX-deficient plasma (FIG. 6A). The thrombin generation was monitored either after induction with tissue factor (FIG. 7a) or after induction with cephalin (FIG. 6B). The efficacy of the antibodies was measured and a dose of 100 μg/ml of antibodies was added during the measurement of thrombin generation. This dose is capable of inhibiting any thrombin generation induced by 1 U/ml of FIX (curve □). In the presence of these antibodies, GPAD-FXa at 0.18 μg/ml (curve ♦) or 0.36 μg/ml (curve ⋄) is capable of restoring a thrombin generation greater than the FIX-deficient plasma having received FIX replacement (1 U/ml; curve ▪), but also than a normal plasma (). As for the FVIII deficiency in the presence of inhibitors, the times to peak are also decreased compared with the controls.

### Example 8

### GPAD-FXa Corrects the Presence of Coagulation Inhibitors in Thrombin Generation

A TGT assay was carried out by adding, to normal plasma, therapeutic doses either of Fondaparinux (1 μg/ml) or of Rivaroxaban (0.35 μg/ml). The thrombin generation is greatly decreased at the inhibitor concentrations used (FIG. 7, curves ⋄ and Δ) compared with the control curve (). The GPAD-FXa supplementation (0.36 μg/ml) makes it possible to significantly restore a part of the thrombin generation (FIG. 7, curves ▪ and ▴). The GPAD-FXa appears to be more effective in correcting the presence of Fondaparinux since several parameters of the TGT are improved (lag time, peak height, area under the curve and velocity). The correction of Rivaroxaban occurs especially with regard to the amount of thrombin generated and not with regard to the kinetics of its appearance. Since the molar concentration of inhibitors is in great excess compared with GPAD-FXa, the correction of the thrombin generation remains partial compared with the control plasma.

### Example 9

### GPAD-FXa Corrects the Presence of Coagulation Inhibitors in Chronometric Tests

The effect of the GPAD-FXa in the presence of the inhibitors Fondaparinux (1 μg/ml) and Rivaroxaban (0.35 μg/ml) was measured using chronometric tests for measuring the prothrombin time (PT) and the activated partial thromboplastin time (aPPT) (FIG. 8). As already demonstrated, the presence of Fondaparinux (1 μg/ml) does not affect the prothrombin time (Smogorzewska A et al. Arch Pathol Lab Med. 2006 130(11):1605-11). Consequently, the effect of the GPAD-FXa cannot be evaluated. The presence of Rivaroxaban (0.35 μg/ml) increases the prothrombin time (from 13.75 to 32.3 s). The presence of GPAD-FXa brings the coagulation time back to 31.2 s, i.e. a decrease of 4.5%.

The presence of Fondaparinux (1 μg/ml) increases the aPPT, which goes from 33.1 s to 37.3 s. The presence of GPAD-FXa makes it possible to normalize the aPPT to 33.5 s in the presence of this inhibitor. The presence of Rivaroxaban (0.35 μg/ml) very significantly increases the prothrombin time (from 33.1 to 63.25 s). The presence of GPAD-FXa makes it possible to decrease it to 56.65 s, i.e. a decrease of 11%. These modest decreases in the effect of the inhibitors correlate with the data obtained in example 8. However, they are obtained with a dose of GPAD-FXa (0.36 μg/ml) that is much lower in terms of molarity than those of the inhibitors (82× and 115× lower, respectively).

### Example 10

### Generation of Optimized Constructs

The GPAD-FXa construct corresponding to the fusion of the truncated light chain of FX to the heavy chain without the addition of any additional sequence is called GPAD1-FXa. This fusion allows the natural formation of a consensus protein sequence corresponding to a furin cleavage site and which separates the two chains. The GPAD-FXa construct containing an additional furin site compared to the protein sequence corresponding to a sequence resulting from fusion between the heavy chain and the truncated light chain is called GPAD2-FXa (FIG. 9). In an attempt to improve the production of these molecules, optimized versions were produced by introducing several modifications: a signal peptide of MB7 replaces the TIMP signal peptide, and the coding sequence was optimized for expression in a eukaryotic system. The resulting two molecules are called GPAD1opt-FXa and GPAD2opt-FXa (FIG. 9).

Moreover, GPAD3opt-FXa and GPAD3-2Fopt-FXa molecule structures were produced either by adding a linker of variable size ending with an arginine upstream of the RKR site of the activation peptide (L1 in GPAD3opt-FXa), or by adding, at the same place, linkers beginning with the sequence RKR at the N-terminal end and ending with an arginine in the C-terminal position (L2 in GPAD3-2Fopt-FXa, FIG. 9). The molecules were transiently expressed in the HEK293F line and the amounts of FX produced were measured by ELISA as described in example 1.

The comparison of the expression of GPAD1-FXa and of GPAD2-FXa shows a slight non-significant advantage for the expression of GPAD2-FXa (FIG. 10A). The comparison of the expression of GPAD2-FXa, GPAD1opt-FXa and GPAD2opt-FXa itself shows a significant advantage in expressing the GPAD2opt-FXa molecule (4.57 μg/ml) instead of 1.25 μg/ml of its non-optimized version (FIG. 10B).

### Example 11

### Chromogenic Activity of GPAD2opt-FXa

GPAD2opt-FXa produced in HEK293 was purified according to the protocol described in the article by Husi et al. J. Chromato 2001. Its chromogenic activity on substrate Pefachrome 8595 was measured as described, and compared to that of a plasma FXa (FIG. 11). The activity of the purified GPAD2opt-FXa is found to be identical to that of the plasma FXa. This result indicates that the enzymatic activity of the GPAD2opt-FXa on a small substrate is identical to that of an activated plasma FX.

### Example 12

### Effect of the Coexpression of Furin on the TGT Activity of GPAD2opt-FXa

The GPAD2opt-FXa was expressed in HEK while optionally overexpressing furin by cotransfection. The activities of the two GPAD2opt-FXas were evaluated in terms of thrombin generation in an FVIII-deficient plasma after induction of coagulation with cephalin (FIG. 12). The GPAD2opt-FXa molecule makes it possible to effectively correct the FVIII deficiency after induction with cephalin (♦). The coexpression with furin makes it possible to obtain a molecule of active GPAD2opt-FXa (⋄) earlier and more intensely than a molecule produced without coexpressing furin (♦). These results show that the presence of furin during the production improves the specific activity of the molecule produced.

### Example 13

### GPAD2opt-FXa Corrects the FVIII Deficiency in Terms of Thrombin Generation

A TGT assay was carried out by adding GPAD2opt-FXa to FVIII-deficient plasma (FIGS. 13A and B). The control plasma (curve ) has an expected TGT activity. The FVIII deficiency (curve ◯) completely abolishes the thrombin generation induced with the TF/PL mixture or with cephalin (panel A and B, respectively). The complementation of the deficient plasma with a therapeutic dose of 1 U/ml of FVIII (curve ▪) makes it possible to obtain a signal very close to the signal obtained with a normal plasma. The supplementation with 0.1 U/ml of recombinant factor VIII (curve □) makes it possible to obtain a signal that is significantly higher than with the control plasma, but lower than the normal. The supplementation of the FVIII-deficient plasma with GPAD2opt-FXa (2 μg/ml, curve ♦) makes it possible to significantly restore the thrombin generation more promptly than with the positive controls, while producing, however, a slightly lower amount of thrombin. This result indicates that the GPAD2opt-FXa makes it possible to restore a significant thrombin generation in the absence of FVIII.

### Example 14

### GPAD2opt-FXa Corrects the FIX Deficiency in Terms of Thrombin Generation

A TGT assay was carried out by adding GPAD2opt-FXa to FIX-deficient plasma (FIGS. 14A and B). The control plasma (curve ) has an expected TGT activity. The FIX deficiency (curve ◯) completely abolishes the thrombin generation induced with the TF/PL mixture or with cephalin (panel A and B, respectively). The complementation of the deficient plasma with therapeutic doses either of 1 U/ml of FIX (curve ▪) or of 0.1 U/ml of plasma factor IX (curve □) makes it possible to obtain a signal greater than the control in the two cases. The GPAD2opt-FXa supplementation (2 μg/ml, curve ♦) makes it possible to significantly restore a part of the thrombin generation. This restoration corresponds virtually to an FIX replacement following induction with TF, whereas it remains less significant after induction with cephalin. This result indicates that the GPAD2opt-FXa makes it possible to restore a significant thrombin generation in the absence of FIX.

### Example 15

### GPAD2opt-FXa does not Correct the FX Deficiency in Terms of Thrombin Generation

A TGT assay was carried out by adding GPAD2opt-FXa to an FX-deficient plasma (FIGS. 15A and B). The control plasma (curve ) has an expected TGT activity. The FX deficiency (curve ◯) completely abolishes the thrombin generation induced with the TF/PL mixture or with cephalin (panel A and B, respectively). The complementation of the deficient plasma with therapeutic doses either of 10 μg/ml of plasma FX (curve ▪) or of 1 μg/ml of plasma factor X (curve □) makes it possible to obtain a signal greater than the control following induction with cephalin. Following an induction with tissue factor, plasma FX at 1 μg/ml is, however, less effective than a normal plasma in generating thrombin. Whatever the inducer, the GPAD2opt-FXa supplementation (2 μg/ml, curve ⋄) does not make it possible to restore thrombin generation. The resulting curve cannot be distinguished from the basal level. This result indicates that the GPAD2opt-FXa, by virtue of its lack of gamma-carboxylated domain, does not make it possible to restore thrombin generation in the absence of FX. This absence of factor X supplementation indicates that the GPAD2opt-FXa will not result in an overdose of factor X which could prove to be thrombogenic. Furthermore, this result makes it possible to confirm that the results obtained in terms of TGT do not correspond to a thrombin generation that would be nonspecific.

### Example 16

### Construction of a GPAD2opt-FXa Molecule Fused to the scFc

The factor X molecule in its activated form has a very short half-life in the circulation (Invanciu et al. Nat. Biotech. 2011). In order to increase the circulating half-life of the GPAD2opt-FXa, chimeric molecules fused to the Fc domain of antibodies in the form of a single chain (scFc) were generated. Various combinations were tested with the GPAD2opt-FXa molecule grafted either in the N-terminal position or in the C-terminal position (FIG. 16). Furthermore, linkers (1) between the scFc domain and the GPAD2opt-FXa portion were added (FIG. 16). The molecules were produced in the HEK line in a manner similar to the GPAD2opt-FXa, then purified on protein A gel in a ratio of 100 μl of gel for 1 mg of protein. They were eluted in 25 mM citrate buffer, pH 3.0, in 4 fractions of 200 μl which are pooled and immediately neutralized with 32 μl of 2M Tris, pH 9.0. The molecules were then dialyzed against 25 mM Hepes buffer, 175 mM NaCl, pH 7.4.

The purified molecules were assayed either by recognition of the FX portion as described in example 1, or by assaying the Fc fraction using the FastELISA kit, ref RDB-3257 (human immunoglobulin G assay, RD-Biotech, France) and by applying the protocol recommended by the supplier. The two assays make it possible to find similar values indicating that the chimeric molecule is effectively recognized by these two methods. Moreover, each of the two components of the molecule must fold in the native manner in order for the two revelations to be functional. The quality of the scFcl-GPAD2opt-FXa molecule is presented in FIG. 17 following a purification step on protein A gel.

### Example 17

### Measurement of the Activity of the scFcl-GPAD2opt-FXa Molecule in FVIII-Deficient Plasma

The scFcl-GPAD2opt-FXa molecule is used as a model for studying the feasibility of a long-lasting molecule. The properties of this purified molecule are compared to those of the GPAD2opt-FXa.

The scFcl-GPAD2opt-FXa molecule was compared with the GPAD2opt-FXa molecule in a TGT test in FVIII-deficient plasma by induction with cephalin (FIG. 18). The chimeric molecule was used in a manner equimolar (4 μg/ml) to the GPAD2opt-FXa (2 μg/ml). The two molecules make it possible to obtain a similar restoration of thrombin generation, indicating that the scFc portion and the linker do not appear to impair the anti-hemophilic function of the protein.

### Example 18

### Measurement of the Activity of the scFcl-GPAD2opt-FXa Molecule in Terms of Chromogenic Activity in the Presence or Absence of Rivaroxaban

The chromogenic activity of the purified scFcl-GPAD2opt-FXa molecule on the substrate Pefachrome 8595 was measured as described in example 1 (FIG. 18). The resulting activity was compared to that of plasma factor Xa. The two molecules have a similar chromogenic activity, indicating that the catalytic site of the scFcl-GPAD2opt-FXa molecule has retained its enzymatic properties. A similar assay was carried out by adding 0.35 μg/ml of Rivaroxaban to the reaction medium. Rivaroxaban inhibits the two molecules with a maximum efficiency since their chromogenic activities after treatment are of the order of the background noise. This result indicates that the scFcl-GPAD2opt-FXa molecule has retained its capacity to be inhibited by Rivaroxaban.

### Example 19

### Measurement of the Antidote Activity of the scFcl-GPAD2opt-FXa Molecule in a TGT Test

A thrombin generation test on a normal plasma optionally containing Rivaroxaban (0.25 μg/ml) was carried out following initiation either with the tissue factor/phospholipids mixture (FIG. 20A) or cephalin (FIG. 20B). At this dose, Rivaroxaban totally inhibits the thrombin generation from this plasma (curve ◯). The addition of an increasing concentration of scFcl-GPAD2opt-FXa (25 or 50 μg/ml) makes it possible, depending on the dose, to restore the formation of a significant amount of thrombin and to improve the formation kinetics. The dose of 50 μg/ml makes it possible to completely normalize the thrombin generation induced with the TF/PL mixture and to very significantly correct the action of the Rivaroxaban following induction with cephalin. These results indicate that the scFcl-GPAD2opt-FXa molecule is an effective antidote to the new oral anticoagulants.

### Example 20

### Effect of the Length of the Activation Peptide on the Structure of FXa

The molecular modeling analysis of the structure of FXa reveals that the length of the activation peptide appears to have an impact on the folding of the protein. Thus, when the activation peptide is deleted (GPAD1 molecule), a strong structural constraint on the folding of the molecule is observed. This constraint is represented by the high value of the RMSD (Root Mean Square Deviation) during the superposition of the structural model GPAD1.M0013 on the crystallographic structure of FXa, 2GD4 (main chain) (table V).

TABLE V
Value of the RMSDs of FX-WT and of the truncated molecules
Various truncated FX molecules were modeled using the structure
of FXa-WT 2GD4 as template and the differences in RMSD were
calculated with respect to the value of the crystalline FXa-WT (2GD4)
following the superposition of the model on the crystallographic structure.

| | | NRES (Number of |
|---|---|---|
| Protein | RMSD (angstrom) | Overlapping Residues) |
| 2GD4 | 0 | 288 |
| GDFXa.M0015 | 0.297 | 288 |
| GPAD1.M0013 | 1.095 | 288 |
| GPAD2.M0025 | 0.526 | 288 |
| GPAD3_GSSG.M0028 | 0.348 | 288 |
| GPAD3_GGS.M0029 | 0.428 | 288 |
| GPAD3_G4S2.M0020 | 0.349 | 288 |
| GPAD3_G4S2.M0006 | 0.331 | 288 |

This value of 1.095 is much higher than that of the 3D model of FXa containing the entire sequence of the activation peptide, GDFXa.M0015 (0.297). The same analyses were carried out on 3D models containing, in place of the sequence of the activation peptide, the linker RKR(GPAD2.M0025), short linkers GSSGR and GGSR (GPAD3_GSSG.M0028 and GPAD3_GGS.M0029) or a long linker GGGGSGGGGSR (SEQ ID No.: 91) (GPAD3_G4S2.M0020 and GPAD3_G4S2.M0006). It was observed that the RKR and GGSR linkers retain a significant structural constraint on folding, although this is lower than that of GPAD 1. On the other hand, the RMSD values of the GSSGR and GGGGSGGGGSR (SEQ ID No.: 91) linkers are low and close to that of the model containing the complete sequence of the activation peptide considered to be non-constrained, indicating that the addition of these two linkers makes it possible not to constrain the folding of the GPAD molecule. The presence of linker could allow better biosynthesis of the protein, better productivity and maintaining of the enzymatic activity.

1.-31. (canceled)

32. A protein comprising SEQ ID No.: 11 or SEQ ID No.: 25 or SEQ ID No.: 26, directly fused, or fused via a linker, to SEQ ID No.: 6.

33. The protein as claimed in claim 32, consisting of SEQ ID No.: 11 or SEQ ID No.: 25 or SEQ ID No.: 26, directly fused, or fused via a linker, to SEQ ID No.: 6.

34. The protein as claimed in claim 32, wherein the linker is -Arg-Lys-Arg-.

35. The protein as claimed in claim 32, wherein the linker is selected from the group consisting of the cleavage sites of activated protein C, kallikrein, FXIIa, FXIa, FXa, FIXa, and FVIIa.

36. The protein as claimed in claim 32, wherein the protein is SEQ ID No.: 7, SEQ ID No.: 9, SEQ ID No.: 21, SEQ ID No.: 22, SEQ ID No.: 23, SEQ ID No.: 24, SEQ ID No.: 60, SEQ ID No.: 61, SEQ ID No.: 62 or SEQ ID No.: 63.

37. The protein as claimed in claim 32, wherein it comprises at least one mutation chosen from a point substitution, a deletion and an insertion.

38. The protein as claimed in claim 37, wherein the mutation is a point substitution.

39. The protein as claimed in claim 37, wherein arginine 138 of SEQ ID No.: 6 is substituted by phenylalanine, glycine, isoleucine or tyrosine.

40. The protein as claimed in claim 37, wherein lysine 82 of SEQ ID No.: 6 is substituted by tyrosine.

41. The protein as claimed in claim 37, comprising an insertion into SEQ ID No.: 11, SEQ ID No.: 25 or SEQ ID No.: 26.

42. The protein as claimed in claim 41, wherein the insertion is a linker selected from:
-GSSG-, -RGSSG-, -GSSGR-, -RKRGSSGR-, -R(GGGGS)n-, -RKR(GGGGS)nR-, -(GGGGS)n-, -(GGGGS)nR-, X, R—X, X—R, and RKR—X—R, wherein n is an integer from 1 to 5, preferably from 1 to 3, X is a peptide of 4 to 52 amino acids, G is glycine, S is serine, R is arginine, and K is lysine.

43. The protein as claimed in claim 37, wherein the insertion into the sequence SEQ ID No.: 25 consists of an insertion, between amino acids 98 and 99, of the linker -GSSG-, -RGSSG-, -R(GGGGS)n-, -(GGGGS)n-, X or R—X, wherein n is an integer from 1 to 5, preferably from 1 to 3, X is a peptide of 4 to 52 amino acids, and G is glycine, S is serine, and R is arginine.

44. The protein as claimed in claim 37, wherein the insertion into the sequence SEQ ID No.: 26 consists of an insertion, between amino acids 97 and 98, of the linker -GSSG-, -RGSSG-, -R(GGGGS)n-, -(GGGGS)n-, X or R—X, wherein n is an integer from 1 to 5, preferably from 1 to 3, X is a peptide of 4 to 52 amino acids, G is glycine, S is serine, and R is arginine.

45. The protein as claimed in claim 37, wherein the insertion into the sequence SEQ ID No.: 25 consists of an insertion, between amino acids 99 and 100, of the linker -GSSGR-, -(GGGGS)nR-, -RKRGSSGR-, -RKR(GGGGS)nR-, X—R or RKR—X—R, wherein n is an integer from 1 to 5, preferably from 1 to 3, X is a peptide of 4 to 52 amino acids, G is glycine, S is serine, R is arginine, and K is lysine.

46. The protein as claimed in claim 37, wherein the insertion into the sequence SEQ ID No.: 26 consists of an insertion, between amino acids 98 and 99, of the linker -GSSGR-, -(GGGGS)nR-, -RKRGSSGR-, -RKR(GGGGS)nR-, X—R or RKR—X—R, wherein n is an integer from 1 to 5, preferably from 1 to 3, X is a peptide of 4 to 52 amino acids, and G is glycine, S is serine, R is arginine, and K is lysine.

47. The protein as claimed in claim 37, wherein the protein is SEQ ID No.: 28, SEQ ID No.: 29, SEQ ID No.: 30, SEQ ID No.: 65, SEQ ID No.: 66 or SEQ ID No.: 67.

48. The protein as claimed in claim 32, wherein the protein is single-stranded.

49. The protein as claimed in claim 32, wherein the protein is fused, in the N-terminal or C-terminal position, to a wild-type Fc fragment or to a wild-type scFc fragment.

50. The protein as claimed in claim 49, wherein the Fc fragment is SEQ ID No.: 39 or SEQ ID No.: 54, optionally followed by a lysine in the C-terminal position.

51. The protein as claimed claim 50, wherein the protein is SEQ ID No.: 32, SEQ ID No.: 33, SEQ ID No.: 34, SEQ ID No.: 35, SEQ ID No.: 36, SEQ ID No.: 68, SEQ ID No.: 69, SEQ ID No.: 70, SEQ ID No.: 71 or SEQ ID No.: 72.

52. The protein as claimed in claim 50, wherein the protein is SEQ ID No.: 37, SEQ ID No.: 38, SEQ ID No.: 73 or SEQ ID No.: 74.

53. A nucleic acid encoding the protein as claimed in claim 32.

54. The nucleic acid as claimed in claim 53, selected from SEQ ID No.: 8, SEQ ID No.: 10, SEQ ID Nos.: 45 to 51, SEQ ID Nos.: 57 to 59 and SEQ ID Nos.: 77 to 90.

55. An expression cassette comprising the nucleic acid as claimed in claim 53.

56. An expression vector comprising the expression cassette as claimed in claim 55.

57. An expression vector comprising the nucleic acid as claimed in claim 53.

58. A recombinant cell comprising the nucleic acid as claimed in claim 53.

59. A recombinant cell comprising the vector as claimed in claim 56.

60. A pharmaceutical composition, comprising the protein as claimed in claim 32 and a pharmaceutically acceptable carrier.

61. A method of treating hemorrhagic disorders, comprising administering to a subject in need thereof an effective amount of the protein as claimed in claim 32.

62. A method of preventing or treating hemorrhagic events induced by taking anticoagulants that are factor Xa-specific inhibitors, comprising administering to a subject in need thereof an effective amount of the protein as claimed in claim 32.

63. A process for producing a protein, comprising:
a) transfecting eukaryotic cells with expression vectors comprising at least one nucleic acid expressing the protein as claimed in claim 32;
b) culturing the transfected eukaryotic cells obtained in a), so as to express the protein; and
c) optionally, purifying the expressed protein.

64. The process as claimed in claim 63, wherein in step a) the eukaryotic cells are also transfected with a vector expressing furin.

65. A process for producing a protein as claimed in claim 32, comprising:
(a) inserting into a non-human mammalian embryo a DNA sequence comprising SEQ ID No.: 8, SEQ ID No.: 10, SEQ ID Nos.: 45 to 51, SEQ ID Nos.: 57 to 59 or SEQ ID Nos.: 77 to 90, said DNA sequence being under the transcriptional control of a mammalian casein promoter or a mammalian whey promoter, said DNA sequence also comprising a signal sequence allowing the secretion of said protein,
(b) leaving said embryo to develop in an adult mammal,
(c) inducing lactation in said mammal or in a female descendent of said mammal in which said DNA sequence, the promoter and the signal sequence are present in the genome of the mammalian tissue,
(d) collecting the milk of said lactating mammal, and
(e) isolating said protein from said collected milk.
